SEQUENCE LISTING (1 sequence)

SEQ ID NO: 1 - DNA, 21 bp, Homo sapiens
cagcagcagagtcttcatcat

(19) United States
(12) Patent Application Publication
(10) Pub. No.: US 2014/0275214 A1
(43) Pub. Date: Sep. 18, 2014

(54) CUSTIRSEN TREATMENT WITH REDUCED TOXICITY

(71) Applicants: Laura Rabinovich-Guilatt, Kfar Saba (IL); Anna Elgart, Mevaseret Zion (IL)

(72) Inventors: Laura Rabinovich-Guilatt, Kfar Saba (IL); Anna Elgart, Mevaseret Zion (IL)

(73) Assignee: TEVA PHARMACEUTICAL INDUSTRIES, LTD., Petach-Tikva (IL)

(21) Appl. No.: 14/207,469

(22) Filed: Mar. 12, 2014

Related U.S. Application Data
(60) Provisional application No. 61/782,451, filed on Mar. 14, 2013.

(51) Int. Cl. A61K 31/7125; C12N 15/113; A61K 31/69; A61K 31/573; A61K 31/4709

(52) U.S. Cl. CPC A61K 31/7125; A61K 31/573; A61K 31/4709; A61K 31/69; C12N 15/113
USPC 514/44 A

(57) ABSTRACT

The present invention provides a method for providing antisense therapy which reduces the expression of clusterin to provide therapeutic benefit in the treatment of cancer, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, to a human subject in need of treatment for the cancer, which human subject also receives at least one chemotherapeutic agent, hormone ablation therapy, or radiation therapy, wherein the anti-clusterin oligonucleotide is administered at least 3 times during a 5 to 9 day period, wherein at least 1 of the administrations is at a dose other than 640 mg. The present invention also provides a method for providing antisense therapy which reduces the expression of clusterin to provide therapeutic benefit in the treatment of myeloma.

This application claims the benefit of U.S. Provisional Application No. 61/782,451, filed Mar. 14, 2013, the contents of which is hereby incorporated by reference in its entirety.

Throughout this application, various publications are referenced, including referenced in parenthesis. Full citations for publications referenced in parenthesis may be found listed in alphabetical order at the end of the specification immediately preceding the claims. The disclosures of all referenced publications in their entireties are hereby incorporated by reference into this application in order to more fully describe the state of the art to which this invention pertains.

### REFERENCE TO SEQUENCE LISTING

This application incorporates-by-reference nucleotide and/or amino acid sequences which are present in the file named “140312_{—}2609_{—}85013_Sequence_Listing_ACK.txt,” which is 1 kilobyte in size, and which was created Mar. 11, 2014 in the IBM-PC machine format, having an operating system compatibility with MS-Windows, which is contained in the text file filed Mar. 12, 2014 as part of this application.

### BACKGROUND OF THE INVENTION

Clusterin is a secretable cytoprotective protein that is upregulated in response to a number of tumor cell killing interventions, specifically chemotherapy, hormone ablation therapy and radiation therapy. As described in U.S. Patent Application Publication No. 2008/0119425, the contents of which are incorporated herein by reference, clusterin is expressed in many malignancies including NSCLC, bladder cancer, ovarian cancer, renal cancer, melanoma, and pancreatic cancer.

Custirsen (also known as, TV-1011, OGX-011, and Custirsen sodium) is a second-generation antisense oligonucleotide (ASO) that inhibits clusterin expression. It has a 2′-MOE modification to the four ribonucleotides on both ends of the 21-mer phosphorothioate backbone. This results in an increased target binding affinity, resistance to degradation, and substantially better tissue PK than first-generation ASOs. The second-generation antisense molecules have a greater affinity for RNA targets and therefore greater potency, as demonstrated by the improved antisense potency observed in cell culture systems and in animals. In addition, the 2′-MOE modification results in decreased binding affinity to RNase H, the principal nuclease that cleaves ASO-bound RNA, which results in significantly improved tissue half-life in vivo (Cleave et al., 2002). This produces a longer duration of action, allowing less frequent dosing (Bennett et al., 2010). Finally, 2′-MOE ASOs have been reported to have a better safety profile than unmodified phosphorothioate ASOs (Henry et al., 2000).

Custirsen is designed specifically to bind to a portion of clusterin mRNA, resulting in the inhibition of the production of clusterin protein. The structure of custirsen is available, for example, in U.S. Pat. No. 6,900,187, the contents of which are incorporated herein by reference. A broad range of studies have shown that custirsen potently reduces the expression of clusterin, facilitates apoptosis, and sensitizes cancerous human prostate, breast, ovarian, lung, renal, bladder, and melanoma cells to chemotherapy (Miyake at al. 2005), see also, U.S. Patent Application Publication No. 2008/0119425 A1, the contents of which are incorporated herein by reference.

Custirsen is being developed for use in combination with chemotherapy, radiotherapy, or hormonal therapy for the treatment of some cancers known to express clusterin (Hare at al., 2001; Custirsen Sodium Investigator's Brochure).

Improved methods for administering custirsen are needed.

### SUMMARY OF THE INVENTION

The present invention provides a method for providing antisense therapy which reduces the expression of clusterin to provide therapeutic benefit in the treatment of cancer, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, to a human subject in need of treatment for the cancer, which human subject also receives at least one chemotherapeutic agent, hormone ablation therapy, or radiation therapy, wherein the anti-clusterin oligonucleotide is administered at least 3 times during a 5 to 9 day period, wherein at least 1 of the administrations is at a dose other than 640 mg.

The present invention provides a method for providing antisense therapy which reduces the expression of clusterin to provide therapeutic benefit in the treatment of myeloma, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-β-methoxyethyl modifications, has nucleotides 5-17 which are 2′ deoxynucleotides, and has 5-methylcytosines at nucleotides 1, 4, and 19, to a human subject in need of treatment for myeloma, which human subject also receives at least one chemotherapeutic agent, hormone ablation therapy, or radiation therapy.

### BRIEF DESCRIPTION OF THE DRAWINGS

FIG. 1: RPMI 8226 tumor volumes

FIG. 2: Body weight changes

FIG. 3: Survival curves

FIG. 4: RPMI-8226 tumor volumes

FIG. 5: Body weight measurements

### DETAILED DESCRIPTION OF THE INVENTION

The present invention provides a method for providing antisense therapy which reduces the expression of clusterin to provide therapeutic benefit in the treatment of cancer, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, to a human subject in need of treatment for the cancer, which human subject also receives at least one chemotherapeutic agent, hormone ablation therapy, or radiation therapy, wherein the anti-clusterin oligonucleotide is administered at least 3 times during a 5 to 9 day period, wherein at least 1 of the administrations is at a dose other than 640 mg.

In some embodiments, the 5-9 day period is a 7 day period.

In some embodiments, the anti-clusterin oligonucleotide is administered on day 1, 3 and 5 of the 7 day period.

In some embodiments, the 7 day period is immediately before the first week of a chemotherapy treatment cycle.

In some embodiments, the anti-clusterin oligonucleotide is administered to the subject 3 times within a 5 to 9 day period and then once weekly thereafter.

In some embodiments, about 640 mg of the anti-clusterin oligonucleotide is administered to the human subject once in the 5 to 9 day period.

In some embodiments, about 640 mg of the anti-clusterin oligonucleotide is administered to the human subject twice in the 5 to 9 day period.

In some embodiments, about 480 mg of the anti-clusterin oligonucleotide is administered to the human subject once in the 5 to 9 day period.

In some embodiments, about 480 mg of the anti-clusterin oligonucleotide is administered to the human subject twice in the 5 to 9 day period.

In some embodiments, about 480 mg of the anti-clusterin oligonucleotide is administered to the human subject 3 times in the 5 to 9 day period.

In some embodiments, about 320 mg of the anti-clusterin oligonucleotide is administered to the human subject once in the 5 to 9 day period.

In some embodiments, about 320 mg of the anti-clusterin oligonucleotide is administered to the human subject twice in the 5 to 9 day period.

In some embodiments, about 320 mg of the anti-clusterin oligonucleotide is administered to the human subject 3 times in the 5 to 9 day period.

In some embodiments, the anti-clusterin oligonucleotide is administered at 1 dose of 320 mg, 1 dose of 480 mg, and 1 dose of 640 mg, in the 5 to 9 day period.

In some embodiments, the first, second and third doses in the 5 to 9 day period are 320, 480, and 640 mg, respectively.

In some embodiments, the doses are administered on days 1, 3 and 5 of the 5 to 9 day period.

In some embodiments, the human subject receives a 320-mg dose of the anti-clusterin oligonucleotide on day 1, a 480-mg dose of the anti-clusterin oligonucleotide on day 3, and a 640-mg dose of the anti-clusterin oligonucleotide on day 5 of the 5 to 9 day period.

In some embodiments, the human subject is less likely to have aPTT prolongation than a corresponding human subject who is administered 640 mg of the anti-clusterin oligonucleotide 3 times in the 5 to 9 day period.

In some embodiments, the human subject is less likely to have tachycardia than a corresponding human subject who is administered 640 mg of the anti-clusterin oligonucleotide 3 times in the 5 to 9 day period.

In some embodiments, the human subject is less likely to have a flu-like symptom compared to a corresponding human subject who is administered 640 mg of the anti-clusterin oligonucleotide 3 times in the 5 to 9 day period.

In some embodiments, the flu-like symptom is fatigue, chills, diarrhea, constipation, nausea, vomiting, cough, insomnia, hot flush, headache, feeling cold, feeling hot, or dizziness.

In some embodiments, the flu-like symptom is upper abdominal pain, arthralgia, asthenia, back pain, chest discomfort, a headache, hyperhidrosis, myalgia, or pyrexia.

In some embodiments, the flu-like symptom is a fever.

In some embodiments, the flu-like symptom is having chills.

In some embodiments, the flu-like symptom is having rigors.

In some embodiments, the flu-like symptom is a headache.

In some embodiments, the flu-like symptom is vomiting.

In some embodiments, the human subject suffers from less aPTT prolongation compared to a corresponding human subject who is administered 640 mg of the anti-clusterin oligonucleotide 3 times in the 5 to 9 day period.

In some embodiments, the human subject suffers from less tachycardia compared to a corresponding human subject who is administered 640 mg of the anti-clusterin oligonucleotide 3 times in the 5 to 9 day period.

In some embodiments, the human subject suffers from a flu-like symptom with reduced severity compared to a corresponding human subject who is administered 640 mg of the anti-clusterin oligonucleotide 3 times in the 5 to 9 day period.

In some embodiments, the flu-like symptom is fatigue, chills, diarrhea, constipation, nausea, vomiting, cough, insomnia, hot flush, headache, feeling cold, feeling hot, or dizziness.

In some embodiments, the flu-like symptom is upper abdominal pain, arthralgia, asthenia, back pain, chest discomfort, a headache, hyperhidrosis, myalgia, or pyrexia.

In some embodiments, the flu-like symptom is a fever.

In some embodiments, the flu-like symptom is having chills.

In some embodiments, the flu-like symptom is having rigors.

In some embodiments, the flu-like symptom is a headache.

In some embodiments, the flu-like symptom is vomiting.

In some embodiments, the anti-clusterin oligonucleotide is administered in an aqueous solution comprising sodium ions.

In some embodiments, the human subject suffers from metastases.

In some embodiments, the human subject suffers from cancer with lymph node involvement.

In some embodiments, the human subject receives at least one chemotherapeutic agent.

In some embodiments, the at least one chemotherapeutic agent is a taxane, a platinum-based chemotherapeutic agent, a topoisomerase inhibitor, a nucleoside analogue, and/or a proteasome inhibitor.

In some embodiments, the at least one chemotherapeutic agent is a taxane chemotherapy.

In some embodiments, the at least one chemotherapeutic agent is a platinum-based chemotherapeutic agent.

In some embodiments, the at least one chemotherapeutic agent is a taxane and a platinum-based chemotherapeutic agent.

In some embodiments, the at least one chemotherapeutic agent is gemcitabine.

In some embodiments, the at least one chemotherapeutic agent is paclitaxel, docetaxel, or cabazitaxel.

In some embodiments, the at least one chemotherapeutic agent is cisplatin or carboplatin.

In some embodiments, the at least one chemotherapeutic agent is paclitaxel and cisplatin.

In some embodiments, the at least one chemotherapeutic agent is paclitaxel and carboplatin.

In some embodiments, the at least one chemotherapeutic agent is cabazitaxel and cisplatin.

In some embodiments, the at least one chemotherapeutic agent is cabazitaxel and carboplatin.

In some embodiments, the at least one chemotherapeutic agent is gemcitabine and cisplatin.

In some embodiments, the chemotherapeutic agent is docetaxel.

In some embodiments, the at least one chemotherapeutic agent is mitoxantrone.

In some embodiments, the human subject is also administered a corticosteroid.

In some embodiments, the corticosteroid is prednisone.

In some embodiments, the cancer is prostate cancer, bladder cancer, breast cancer, ovarian cancer, lung cancer, renal cancer, melanoma, myeloma, or pancreatic cancer.

In some embodiments, the cancer is lung cancer.

In some embodiments, the lung cancer is other than non-small cell lung cancer (NSCLC).

In some embodiments, the lung cancer is NSCLC.

In some embodiments, the lung cancer is unresectable, advanced or metastatic lung cancer.

In some embodiments, the NSCLC is stage IV lung cancer.

In some embodiments, the NSCLC is of non-squamous histology.

In some embodiments, the NSCLC is lung adenocarcinoma or lung large cell carcinoma.

In some embodiments, the cancer is prostate cancer.

In some embodiments, the prostate cancer is castration-resistant prostate cancer (CRPC).

In some embodiments, the cancer is breast cancer.

In some embodiments, the method further comprises administering a corticosteroid to the human subject a corticosteroid.

In some embodiments, the corticosteroid is dexamethasone.

In some embodiments, a combination of 320 mg of the anti-clusterin oligonucleotide and the corticosteroid is administered to the human subject on day 1, 480 mg of the anti-clusterin oligonucleotide and the corticosteroid is administered to the human subject on day 3 and 640 mg of the anti-clusterin oligonucleotide the corticosteroid is administered to the human subject on day 5 of the 5-9 day period.

In some embodiments, a combination of 320 mg of the anti-clusterin oligonucleotide and 5 mg dexamethasone is administered to the human subject on day 1, 480 mg of the anti-clusterin oligonucleotide and 5 mg dexamethasone is administered to the human subject on day 3 and 640 mg of the anti-clusterin oligonucleotide and 3 mg dexamethasone is administered to the human subject on day 5 of the 5-9 day period.

In some embodiments, dexamethasone is administered intravenously as a bolus 1 hour prior to custirsen administration.

The present invention provides a method for providing antisense therapy which reduces the expression of clusterin to provide therapeutic benefit in the treatment of myeloma, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, to a human subject in need of treatment for myeloma, which human subject also receives at least one chemotherapeutic agent, hormone ablation therapy, or radiation therapy.

In some embodiments, the human subject also receives at least one chemotherapeutic agent, hormone ablation therapy, or radiation therapy.

In some embodiments, the anti-clusterin oligonucleotide is administered at least 3 times during a 5 to 9 day period, wherein at least 1 of the administrations is at a dose other than 640 mg.

In some embodiments, the anti-clusterin oligonucleotide is administered at 1 dose of 320 mg, 1 dose of 480 mg, and 1 dose of 640 mg, in the 5 to 9 day period.

In some embodiments, the first, second and third doses in the 5 to 9 day period are 320, 480, and 640 mg, respectively.

In some embodiments, the doses are administered on days 1, 3 and 5 of the 5 to 9 day period.

In some embodiments, the human subject receives a 320-mg dose of the anti-clusterin oligonucleotide on day 1, a 480-mg dose of the anti-clusterin oligonucleotide on day 3, and a 640-mg dose of the anti-clusterin oligonucleotide on day 5 of the 5 to 9 day period.

In some embodiments, the human subject also receives at least one chemotherapeutic agent.

In some embodiments, the chemotherapeutic agent is a proteasome inhibitor.

In some embodiments, the proteasome inhibitor is bortezomib.

In some embodiments, 640 mg of the anti-clusterin oligonucleotide is administered to the human subject after the 5 to 9 day period.

Aspects of the invention relate to the use of an anti-clusterin oligonucleotide in combination of chemotherapy, radiation therapy, or hormone ablation therapy for the treatment of cancer, for example prostate cancer, bladder cancer, ovarian cancer, renal cancer, melanoma, myeloma, breast cancer, lung cancer, and pancreatic cancer. Non-limiting examples of cancer treatment with anti-clusterin oligonucleotides are described in U.S. Pat. Nos. 7,534,773, 7,569,551, 6,900,187, and 7,285,541, and U.S. Patent Application Publication Nos. 2011/0142827, 2008/119425, and 2013/0017272, the entire contents of each of which are hereby incorporated herein by reference.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

Examples of chemotherapeutic agents whose effectiveness may be enhanced in embodiments of the invention include but are not limited to taxanes (paclitaxel, docetaxel and cabazitaxel, for example), vinca alkaloids (vinblastine, vincristine, vindesine and vinorelbine, for example), antimetabolites (purine analogues, pyrimidine analogues and antifolates, for example), alkylating antineoplastic agents (cylcophosphamide, for example), topoisomerase inhibitors (camptothecin derivatives such astopotecan and irinotecan, for example), angiogenesis inhibitors (bevacizumab, for example), platinum-based therapies (cisplatin, carboplatin and 254-S, for example) differentiation agents (all-trans-retinoic acid (ATRA), for example) and proteasome inhibitors (bortezomib, for example).

### Taxanes

Taxanes are a class of chemotherapeutic including paclitaxel, docetaxel, baccatin III, baccatin V, taxol B (cephalomannine), taxol C, taxol D, taxol E, taxol F, taxol G, cabazitaxel, larotaxel, ortataxel (14 beta-hydroxydeacetyl baccatin III), tesetaxol, 10-deacetyl baccatin III, 7-xylosyl-10-deacetyl cephalomannine, 7-xylosyl-10-deacetyl paclitaxel, 10-deacetyl cephalomannine, 7-xylosyl-10-deacetyl taxol C, 10-deacetyl paclitaxel, 7-xylosyl paclitaxel, 10-deacetyl taxol C, 10-deacetyl-7-epi cephalomaunine, 7-xylosyl taxol C, 10-deacetyl-7-epipaclitaxel, 7-epi cephalomaunine, 7-epi paclitaxel, 7-O-methylthiomethyl paclitaxel, 7-deoxy docetaxel, taxanime M, PG-paclitaxel, DHA-paclitaxel.

Taxanes have been approved by the FDA and include paclitaxel (e.g., for NSCLC, AIDS-related Kaposi sarcoma, breast cancer and ovarian cancer), cabazitaxel (e.g., for prostate cancer), and docetaxel (e.g., for NSCLC, breast cancer, gastric (stomach) cancer, prostate cancer, squamous cell carcinoma of the head and neck).

Taxanes also include derivatives of these compounds, particularly ester and ether derivatives and pharmaceutically acceptable salts thereof. Taxanes may also include any drug or derivative of a drug which has a carbon framework substantially identical to the framework of the above taxanes.

Without being bound to any particular theory, taxanes may achieve their therapeutic effect by interfering with cell division by stabilizing tubulin in the microtubule. Taxanes may be naturally occurring, semi-synthetic, or synthetic compounds. Semi-synthetic taxanes may be prepared by modification of a known or naturally occurring taxane. The taxanes may be prepared as a fatty acid-bound, peptide-bound, albumin-bound or other protein-bound suspension or dissolved in a solution, such as polyoxyl 35 or polysorbate 80.

Paclitaxel

Paclitaxel is sold under the brand names Taxol® and Abraxane®, and has been used for the treatment of lung cancer (including NSCLC), head and neck cancer, ovarian cancer, breast cancer, and Kaposi's sarcoma (Taxol® Package Insert, Bristol-Myersw Squibb Company (Princeton, N.J., USA); D'Addario et al., 2010; National Comprehensive Cancer Network Clinical Practice Guidelines in Oncology, Non-Small Cell Lung Cancer, V.2.2010).

Paclitaxel is known to cause several side effects. Neutropenia, the most frequent side effect, is profound but generally of short duration. Peripheral neuropathy, myalgia, and arthralgia are usually noted with the administration of higher doses of paclitaxel 175 mg/m²) for several cycles. Paclitaxel can cause rapid and complete alopecia. Other toxicities include: mild to moderate nausea, vomiting, diarrhea, and mucositis.

For paclitaxel therapy, standard steroid premedication to prevent severe hypersensitivity reactions and antiemetics may be given according to institutional practice. According to the package insert, the recommended premedication consists of dexamathasone 20 mg p.o. administered twice, approximately 12 and 6 hours before paclitaxel, diphenhydramine (or its equivalent) 50 mg i.v./.p.o. 30 to 60 minutes prior to paclitaxel, and cimetidine (300 mg) or ranitidine (50 mg) i.v./p.o. 30 to 60 minutes prior to paclitaxel.

Docetaxel

Docetaxel is sold under the brand name Taxoter® and has been used for second-line treatment of NSCLC (Taxotere® Prescribing Information, Sanofi-Aventis LLC, May 2010, (Bridgewater, N.J., USA), Docetaxel has also been used as treatment for metastatic breast cancer, early-stage breast cancer and metastatic androgen independent prostate cancer.

Docetaxel is known to cause several side effects, the most common of which are infections, neutropenia, anemia, febrile neutropenia, hypersensitivity, thrombocytopenia, neuropathy, dysgeusia, dyspnea, constipation, anorexia, nail disorders, fluid retention, asthenia, pain, nausea, diarrhea, vomiting, mucositis, alopecia, skin reactions and myalgia.

Neutropenia (<2,000 neutrophils/mm3) occurs in virtually all patients given 60-100 mg/m² of Docetaxel and grade 4 neutropenia (<500 cells/mm³) occurs in 85% of patients given 100 mg/m² and 75% of patients given 60 mg/m².

The incidence of treatment-related mortality associated with Docetaxel therapy is increased in patients with abnormal liver function, in patients receiving higher doses, and in patients with non-small cell lung carcinoma and a history of prior treatment with platinum-based chemotherapy who receive Taxotere® as a single agent at a dose of 100 mg/m².

Patients may be premedicated with corticosteroids, such as dexamethasone, to each Docetaxel administration to reduce the incidence of and severity of fluid retention.

Docetaxel may be prescribed as a one-hour infusion every three weeks or as weekly administration (John D. Hainsworth, “Practical Aspects of Weekly Docetaxel Administration Schedules” September 2004, vol. 9, no. 5, 538-545)

Platinum-Based Chemotherapeutic Agents

Platinum-based chemotherapeutic agents are a class of chemotherapy drugs. Platinum-based chemotherapeutic agents include cisplatin, carboplatin (also known as paraplatin), nedaplatin, oxaliplatin, triplatin tetranitrate, satraplatin, iproplatin, lobaplatin, picoplatin and combinations thereof. Platinum-based chemotherapeutic agents are approved by the FDA and include cisplatin (NSCLC, bladder cancer, cervical cancer, malignant mesothelioma, ovarian cancer, squamous cell carcinoma of the head and neck, and testicular cancer), oxaliplatin (colorectal cancer and stage III colon cancer), and carboplatin (NSCLC and ovarian cancer) are approved by the FDA.

Platinum-based chemotherapeutic agents also include derivatives of these compounds, particularly ester and ether derivatives and pharmaceutically acceptable salts thereof. Platinum-based chemotherapeutic agents may also include any drug or derivative of a drug which has a carbon framework substantially identical to the framework of the above platinum-based chemotherapeutic agents.

Without being bound to any particular theory, platinum-based chemotherapeutic agents can be classified as alkylating or alkylating-like agents because they interact with DNA irreversibly through cross-linking and platinum-DNA adduct forming reactions which prevent DNA repair or replication and result in apoptosis of cells.

Common side-effects of platinum-based chemotherapeutic agents include nephrotoxicity, neurotoxicity, nausea and vomiting, ototoxicity, electrolyte disturbance, myelotoxicity, and hemolytic anemia.

Carboplatin

Carboplatin is sold under the brand name Paraplatin®, and has been used for the treatment of NSCLC (Carboplatin Package Insert, Bedford Labs (Bedford, Ohio, USA); D'Addario et al., 2010; National Comprehensive Cancer Network Clinical Practice Guidelines in Oncology, Non-Small Cell Lung Cancer, V.2.2010).

Bone marrow suppression is the major dose-limiting toxicity of carboplatin. Nausea, vomiting, and loss of appetite are usually mild to moderate. Less common adverse events includes ototoxicity, nephrotoxicity, neurotoxicity, hypomagnesemia, edema, alopecia, amenorrhea, CNS toxicity (dizziness, blurred vision), hypercalcemia, abnormal liver function tests, allergic reactions, and veno-occlusive disease. For full safety information, please refer to the carboplatin package insert, a copy of which is incorporated herein by reference.

### TERMS

As used herein, and unless stated otherwise, each of the following terms shall have the definition set forth below.

As used herein, “about” in the context of a numerical value or range means±10% of the numerical value or range recited or claimed, unless the context requires a more limited range.

As used herein, “anti-clusterin therapy” is therapy which reduces the expression of clusterin. An anti-clusterin therapy may be an anti-clusterin oligonucleotide.

Antisense oligonucleotides (ASOs) are stretches of single-strand deoxyribonucleic acid (DNA) complementary to messenger ribonucleic acid (mRNA) regions of a target gene. Because cellular ribosomal machinery translates mRNA into proteins, expression of specific proteins can be reduced by blocking or reducing this translation.

As used herein, “anti-clusterin oligonucleotide” refers to an antisense oligonucleotide which reduces clusterin expression, and comprises a nucleotide sequence that is complementary to clusterin-encoding mRNA. An example of an anti-clusterin oligonucleotide is custirsen.

As used herein, “custirsen” refers to an anti-clusterin oligonucleotide having nucleotides in the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. Custirsen can be in the form of Custirsen Sodium.

Antisense therapeutics are based on the premise that sequences of single-stranded nucleic acids will bind to complementary strands of nucleic acids through hybridization. A cancer cell with over expression of a specific protein produces an abundance of mRNA that is translated into excess protein. The introduction of a specific complementary or antisense strand of single stranded DNA can bind to the abundant mRNA strands, leading to degradation before translation can occur and reduction in protein levels of the target gene (Crooke 2007).

Second-generation chemistry, used by custirsen sodium, applies 2′ O (2-methoxyethyl) (2′ MOE modification) at the 2 position of the carbohydrate moiety on both ends of the oligonucleotide, resulting in increased target binding affinity, resistance to degradation, and substantially better tissue pharmacokinetics. The improved affinity of custirsen sodium is primarily attributable to its design and composition. In particular, second-generation drugs are composed of both RNA-like and deoxyribonucleic acid (DNA)-like nucleotides, while first generation drugs are entirely DNA like. Because RNA hybridizes more tightly to RNA than to DNA, the second-generation drugs have a greater affinity for RNA targets and therefore greater potency, as demonstrated by the improved antisense potency observed in cell culture systems and in animals. In addition, the 2′ MOE modification results in decreased binding affinity to RNaseH, the principal nuclease that cleaves ASO-bound RNA, which results in significantly improved tissue half-life in vivo. This produces a longer duration of action, allowing less frequent dosing, and has been reported to result in a more attractive safety profile than using unmodified phosphorothioate ASOs.

As used herein, “a human patient afflicted with” a condition, e.g. cancer, means a human patient who was been affirmatively diagnosed to have the condition.

“Combination” with respect to custirsen means either at the same time and frequency, or more usually, at different times and frequencies as custirsen, as part of a single treatment plan. Aspects of the invention include the administration of custirsen before, after, and/or during the administration of a chemotherapeutic agent. Some embodiments of the invention include the administration of custirsen before, after, and/or during the administration of a taxane and/or a platinum-based chemotherapy. A taxane and a platinum-based chemotherapeutic agent may therefore be used, in combination with custirsen according to the invention, but yet be administered at different times, different dosages, and at a different frequency, than custirsen and/or each other. Aspects of the invention also include the administration of custirsen before, after, and/or during the administration of a taxane and/or a platinum-based chemotherapeutic agent. A taxane and/or a platinum-based chemotherapeutic agent may therefore be used, in combination with custirsen according to the invention, but yet be administered at different times, different dosages, and at a different frequency, than custirsen and/or each other. For example, paclitaxel and carboplatin may be used, in combination with custirsen according to the invention, but yet be administered at different times, different dosages, and at a different frequency, than custirsen and/or each other. As another example, docetaxel may be used, in combination with custirsen according to the invention, but yet be administered at different times, different dosages, and at a different frequency, than custirsen and/or each other.

As used herein, “lung adenocarcinoma” encompasses any malignant epithelial NSCLC which has glandular and/or duct differentiation, and excludes any NSCLC that is not predominantly non-squamous. Non-limiting examples of subdivisions of the lung adenocarcinoma subtype of NSCLC are acinar, papillary, BAC, and solid adenocarcinoma with mucin production. One of skill in the art will recognize that lung adenocarcinomas comprising combinations of two or more of these or other subdivisions are common.

As used herein, “lung large cell carcinoma” means a NSCLC of non-squamous histology that is not lung adenocarcinoma.

One of skill in the art will realize that NSCLC, as well as its subtypes, including lung adenocarcinoma and lung large cell carcinoma, are heterogeneous with multiple histological variants. Therefore, the term “non-small cell lung cancer of non-squamous histology” encompasses all types and subdivisions of NSCLC that are predominantly non-squamous.

As used herein, “Stage IV non-small cell lung cancer” means NSCLC comprising a tumor, wherein i) the NSCLC has metastasized to another region of the body outside the lungs or to a contralateral lobe of the lungs, and/or ii) there is malignant pleural effusion, malignant pericardial effusion, and/or a pleural nodule.

As used herein, “effective” when referring to an amount of a taxane, a platinum-based chemotherapeutic agent, custirsen, paclitaxel, docetaxel, or carboplatin, or any combination thereof refers to the quantity of custirsen, chemotherapeutic agent, or combination thereof that is sufficient to yield a desired therapeutic response without undue adverse side effects (such as toxicity, irritation, or allergic response) commensurate with a reasonable benefit/risk ratio when used in the manner of this invention.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. An example of a pharmaceutically acceptable carrier is a nanoparticle. The nanoparticle may be a protein, such as albumin. A taxane, such as Docetaxel or Paclitaxel, and/or a platinum-based chemotherapeutic agent, such as carboplatin, may be conjugated to a nanoparticle. An example of a taxane bound to a nanoparticle includes, but is not limited to nanoparticle paclitaxel (nab-paclitaxel), which is sold under the brand name Abraxane®.

In some embodiments of the invention, the anti-tumor activity of the taxane regimen is enhanced when combined with custirsen-induced clusterin suppression. In some embodiments of the invention, the anti-tumor activity of the taxane/platinum-based chemotherapeutic agent regimen is enhanced when combined with custirsen-induced clusterin suppression. In some embodiments of the invention, the anti-tumor activity of the paclitaxel/carboplatin regimen is enhanced when combined with custirsen-induced clusterin suppression. In some embodiments of the invention, the anti-tumor activity of a docetaxel regimen is enhanced when combined with custirsen-induced clusterin suppression. Since suppressing clusterin expression may in turn lead to increased apoptosis, custirsen has effect on disease progression and survival in cancer as described herein.

The following abbreviations are used herein.

| Abbreviation | Term |
|---|---|
| ADME | absorption, distribution, metabolism, and excretion |
| Ae_{0-XXh} | Amount of the Parent and the Total ASO Excreted |
| | in Urine within Each 24-hour Collection Interval |
| | (such as 0 to 24 hours, 24 to 48 hours, and so on), |
| | XX represented 24-Hour Intervals |
| Ae | Cumulative Amount of the Parent and the Total ASO |
| | Excreted in Urine |
| AE(s) | Adverse Event(s) |
| AIC | Akaike information criterion |
| ALCL | anaplastic large cell lymphoma |
| ALP | alkaline phosphatase |
| ALT | alanine aminotransferase (SGPT) |
| ANC | absolute neutrophil count |
| ANOVA | analysis of variance |
| aPTT | activated partial thromboplastin time |
| ASO | antisense oligonucleotide |
| AST | aspartate aminotransferase (SGOT) |
| AUC₀₋₄₈ | Area Under the Plasma Concentration-Versus-Time |
| | Curve from Time 0 to 48 Hours |
| AUC_{0-inf} | Area Under the Plasma Concentration-Versus-Time |
| | Curve from Time 0 to Infinity |
| AUC₀₋ₜ | Area Under the Plasma Concentration-Versus-Time |
| | Curve from Time 0 to the time of the last measurable |
| | drug concentration |
| AOC%extrap | Percentage of the AUC_{0-inf} Extrapolated from |
| | the Time of the Last Quantifiable Concentration |
| | to Infinite Time |
| AV | atrioventricular |
| BMI | body mass index |
| bpm | beats per minute |
| BQL | below the quantifiable limit |
| BUN | blood urea nitrogen |
| CCDS | Company Core Data Sheet |
| CFR | Code of Federal Regulations |
| CI | confidence interval |
| CIOMS | Council for International Organizations of Medical |
| | Sciences |
| CL | total plasma clearance |
| CLᵣ | renal clearance |
| Cₘₐₓ | maximum observed plasma drug concentration |
| CPP | clinical project physician |
| CRF | case report form (refers to any media used to |
| | collect study data (i.e., paper or electronic)) |
| CRO | contract research organization |
| CRPC | castrate-resistant prostate cancer |
| CRU | clinical research unit |
| CS | compound symmetry |
| CSC | Clinical Supply Chain |
| CTCAE | Common Terminology Criteria for Adverse Events |
| CV % | Percent Coefficient of Variation |
| CYP | cytochrome P450 |
| DDI | drug-drug interaction |
| DNA | deoxyribonucleic acid |
| ECG | electrocardiography, electrocardiogram |
| eCRF | electronic case report form |
| EFD | embryo-fetal development |
| ELFT | elevated liver function test(s) |
| eRT | eResearch Technology |
| EU | European Union |
| FDA | US Food and Drug Administration |
| fₑₓ | fraction of administered dose excreted in urine |
| | after 168 hours |
| GCP | Good Clinical Practice |
| GGT | gamma-glutamyl transpeptidase, gamma-glutamyl |
| | transferase |
| HBsAg | hepatitis B surface antigen |
| HCV | human chorionic virus |
| HIV | human immunodeficiency virus |
| HV(s) | healthy volunteer(s) |
| ICF | informed consent form |
| ICH | International Conference on Harmonization |
| IEC | Independent Ethics Committee |
| INR | International Normalized Ratio |
| IRB | Institutional Review Board |
| IRT | interactive response technology |
| iv | intravenous |
| Kₑₗ | Apparent First-order Terminal Rate Constant |
| | Calculated from a Semi-log Plot of the Plasma |
| | Concentration-Versus-Time Curve |
| LCM | local clinical management |
| LDH | lactate dehydrogenase |
| LFT | liver function test(s) |
| LLOQ | lower limit of quantitation |
| LSO | local safety officer |
| max | maximum |
| mCRPC | metastatic castrate-resistant prostate cancer |
| MCHC | mean corpuscular hemoglobin concentration |
| MCV | mean corpuscular volume |
| MedDRA | Medical Dictionary for Regulatory Activities |
| MOE | methoxytheyl |
| mRNA | messenger ribonucleic acid |
| NA | North America(n) |
| NHT | neoadjuvant hormone therapy |
| NOAEL | no observed adverse effect level |
| NCI CTCAE | National Cancer Institute Common Terminology |
| | Criteria for Adverse Events |
| NSAIDs | nonsteroidal anti-inflammatory drugs |
| NSCLC | non-small cell lung cancer |
| OTC | over-the-counter |
| PCS | potential clinical significance |
| PD | pharmacodynamics |
| PI | principal investigator |
| PK | pharmacokinetic(s) |
| PPD | purified protein derivative |
| PT | prothrombin time |
| q24h | Every 24 Hours |
| QTc | QT Interval Corrected for Heart Rate |
| QTcB | QT Interval Corrected for Heart Rate using the |
| | Bazett Formula |
| QTcF | QT Interval Corrected for Heart Rate using the |
| | Fredericia Formula |
| QWBA | quantitative whole body autoradiography |
| RBC | red blood cell |
| SAE(s) | Serious Adverse Event(s) |
| SAP | Statistical Analysis Plan |
| sCLU | secreted isoform of clusterin |
| SD | standard deviation |
| SDV | source document verification |
| SE | standard error |
| SOC | system organ class |
| SOP | standard operating procedure |
| SPC | Summary of Product Characteristics |
| SUSAR | suspected unexpected serious adverse reaction |
| t_{1/2} | terminal elimination half-life |
| TB | tuberculosis |
| tₘₐₓ | time to maximum observed drug concentration |
| TQT | Thorough QT |
| TSH | thyroid stimulating hormone |
| UDS | urine drug screen |
| ULN | upper limit of the normal range |
| US(A) | United States (of America) |
| USP | United States Pharmacopoeia |
| V_{z} | Apparent Volume of Distribution during the Terminal |
| | Phase |
| WBC | white blood cell |
| WHO | World Health Organization |
| WHO Drug | World Health Organization (WHO) drug dictionary |

Dosage Units

Administration of custirsen can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978, which are incorporated herein by reference. In general, custirsen is administered by intravenous (i.v.), intraperitoneal (i.p.), subcutaneous (s.c.), or oral routes, or direct local tumor injection. In some embodiments, custirsen is administered by i.v. injection.

The amount of custirsen administered may be from 40 to 640 mg, or from 300 to 640 mg. Administration of custirsen may be once in a seven day period, 3 times a week, or more specifically on days 1, 3 and 5, or 3, 5 and 7 of a seven day period. In some embodiments, administration of the antisense oligonucleotide is less frequent than once in a seven day period. In some embodiments, administration of the antisense oligonucleotide is more frequent than once in a seven day period. Dosages may be calculated by patient weight, and therefore in some embodiments a dose range of about 1-20 mg/kg, or about 2-10 mg/kg, or about 3-7 mg/kg, or about 3-4 mg/kg could be used. This dosage is repeated at intervals as needed. One clinical concept is dosing once per week with 3 loading doses during week one of treatment. The amount of antisense oligonucleotide administered is one that has been demonstrated to be effective in human patients to inhibit the expression of clusterin in cancer cells.

A dosage unit may comprise a single compound or mixtures of compounds thereof. A dosage unit can be prepared for oral, injection, or inhalation dosage forms.

In some embodiments, custirsen may be formulated at a concentration of 20 mg/mL as an isotonic, phosphate-buffered saline solution for IV administration. In some embodiments, a formulation of custirsen may comprise 1, 2, 3, 4, 5, 6, 7, 8, 9, or 10% dextrose. In some embodiments, the formulation of custirsen may comprise 5% dextrose. In some embodiments custirsen may be supplied as a 32 mL solution containing 640 mg custirsen sodium in a single vial, or may be supplied as an 8 mL solution containing 160 mg custirsen sodium in a single vial. The drug product and active ingredient of custirsen sodium is a second-generation, 4-13-4 MOE-gapmer antisense oligonucleotide (ASO).

In some embodiments, custirsen may be added to 250 mL 0.9% sodium chloride (normal saline). In some embodiments, the dose may be administered using either a peripheral or central indwelling catheter intravenously as an infusion over 2 hours. Additionally, in some embodiments an infusion pump may be used.

In some embodiments, using proper aseptic technique, two, three, or four 8-mL vials of 20 mg/mL custirsen (for a total of 320, 480, or 640 mg, respectively) may added to 0.9% sodium chloride (normal saline) up to a final volume of 250 mL, as close to the time of administration as possible.

In some embodiments a proper infusion pump rate is set to allow for overfill in the normal saline bag and in the iv tubing to ensure the administration of the entire dose over the 2-hour infusion.

In some embodiments, subjects may receive paclitaxel 200 mg/m² as a constant rate infusion on Day 1 of each of one or more 21-day treatment cycles. The amount of paclitaxel administered may be from 100-250 mg/m². The amount of paclitaxel administered may be 100 mg/m², 105 mg/m², 110 mg/m², 115 mg/m², 120 mg/m², 125 mg/m², 130 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 220 mg/m², 225 mg/m², 230 mg/m², 235 mg/m², 240 mg/m², 245 mg/m², or 250 mg/m². The duration of paclitaxel constant rate infusion may be from 1 to 3 hours, or from 3 to 6 hours. The duration of paclitaxel constant rate infusion may be 1, 1.5, 2, 2.5, 3, 3.5, 4, 4.5, 5, 5.5, or 6 hours. In some embodiments, subjects may receive IV carboplatin at a dose calculated for a target AUC of 6 mg/mL per min as a 30 minute constant rate infusion. The amount of carboplatin may be a dose calculated for a target AUC from 2-8 mg/mL per min. The amount of carboplatin may be a dose calculated for a target AUC of 2 mg/mL per min, 3 mg/mL per min, 4 mg/mL per min, 6 mg/mL per min, 7 mg/mL per min, or 8 mg/mL per min. In some embodiments paclitaxel and/or carboplatin may be administered less frequently than once every 21-days. In some embodiments paclitaxel and/or carboplatin may be administered more frequently than once every 21-days. In some embodiments the carboplatin is administered immediately following paclitaxel. In some embodiments the paclitaxel is administered immediately following the carboplatin.

In some embodiments, a chemotherapeutic agent may be administered via an infusion control device (pump) using non-PVC tubing and connectors.

The pharmacokinetic (area under the time concentration curve [AUC]) and the pharmacodynamic effects (hematologic toxicity) of carboplatin are better predicted by glomerular filtration rate (GFR) based dosing as compared with the more traditional body surface area (BSA) dosing method. The Calvert formula provides a consistent method for determining carboplatin dosage in adults that should produce the desired degree of toxicity (Calvert et al., 1989).

The Calvert formula may be used to calculate the carboplatin dose:

Carboplatin dose(mg)=target AUC×(GFR+25)

The Cockcroft-Gault formula may be used to calculate the creatinine clearance (CrCl) (Cockcroft and Gault, 1976), which can be substituted for glomerular filtration rate (GFR) in the Calvert formula. Calculations may be based upon the serum creatinine value obtained within 72 hours prior to treatment for each cycle.

$$\frac{( 140 - subject ’ s age ) \times subject ’ s actual body weight in {kg}^{*}}{72 \times subject ’ s serum creatinine ( in mg / dL )}$$

*For females, multiply the result by 0.85

In some embodiments doses of chemotherapeutic agents may be based on the subject's actual body weight within 3 days prior to treatment. The same weight measurement may be used to calculate the dosage of both drugs.

In some embodiments, subjects may receive docetaxel 75 mg/m² as an infusion on Day 1 of each of one or more 21-day treatment cycles. The amount of docetaxel administered may be from 25 mg/m² to 100 mg/m². The amount of docetaxel administered may be about 25 mg/m², 30 mg/m², mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m² or 100 mg/m². The duration of docetaxel infusion may be from 1 to 3 hours, or from 3 to 6 hours. The duration of docetaxel constant rate infusion may be 1, 1.5, 2, 2.5, 3, 3.5, 4, 4.5, 5, 5.5, or 6 hours. In some embodiments, docetaxel infusion is constant rate infusion.

In some embodiments, subjects may receive a taxane 75 mg/m² as an infusion on Day 1 of each of one or more 21-day treatment cycles. In some embodiments, subjects may receive a taxane 200 mg/m² as an infusion on Day 1 of each of one or more 21-day treatment cycles. The amount of taxane administered may be from 25 mg/m² to 250 mg/m². The amount of taxane administered may be about 25 mg/m², 30 mg/m², 35 mg/m², 40 mg/m², 45 mg/m², 50 mg/m², 55 mg/m², 60 mg/m², 65 mg/m², 70 mg/m², 75 mg/m², 115 mg/m², 120 mg/m², 125 mg/m², 130 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 220 mg/m², 225 mg/m², 230 mg/m², 235 mg/m², 240 mg/m², 245 mg/m², or 250 mg/m². The duration of taxane infusion may be from 1 to 3 hours, or from 3 to 6 hours. The duration of taxane constant rate infusion may be 1, 1.5, 2, 2.5, 3, 3.5, 4, 4.5, 5, 5.5, or 6 hours. In some embodiments, taxane infusion is constant rate infusion.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Administration of Custirsen to Healthy Volunteers

In a Phase I Healthy Volunteer study with custirsen (TV1011-DDI-102), subjects either received a single dose of 640 mg custirsen with a premedication regimen of 400 mg ibuprofen or repeated doses of 640 mg custirsen with premedication regimen of 650 mg acetaminophen.

Constitutional symptoms were experienced by subjects, including upper abdominal pain, arthralgia, asthenia, back pain, chest discomfort, chills, dizziness, feeling cold, feeling hot, headaches, hyperhidrosis, myalgia, nausea, pruritus, pyrexia, and vomiting. Symptoms were only partially alleviated by ibuprofen or acetaminophen administration. Three subjects withdrew from the study due to these constitutional symptoms. No deaths or other SAES were reported.

### Example 2

### Administration of Ascending Custirsen Doses (320, 480, and 640 mg)

Selection of Drugs and Dosages

The doses that are administered in this double-blind study (i.e., 320, 480, and 640 mg/day) are within the range of those being evaluated in Phase 3 studies.

Study Objectives

Objectives of the study are as follows:
to assess the pharmacokinetic profile of custirsen following administration of ascending custirsen doses (320, 480, and 640 mg)
to assess the safety and tolerability of custirsen in healthy men as assessed by the following throughout the study:
   adverse events and concomitant medication usage
   clinical laboratory tests
   vital signs measurements
   physical examination findings

Study Design

General Design

This is a 3-arm, parallel-group, active- and placebo-controlled, double-blind, randomized study, to compare treatment with intravenous custirsen at 640 mg (highest intended therapeutic dose) with placebo as a negative control.

On days 1, 3, 5, and 7, subjects are administered their respective treatments as follows:

The 2 groups receive treatment as follows:
group 1—ascending loading doses with investigational product (custirsen):
   320 mg custirsen on day 1
   480 mg custirsen on day 3
   640 mg custirsen on day 5
group 2—high loading doses with investigational product (custirsen):
   640 mg custirsen on day 1
   640 mg custirsen on day 3
   640 mg custirsen on day 5
group 3—placebo (normal saline):
   placebo+5 mg dexamethasone on day 1
   placebo+5 mg dexamethasone on day 3
   placebo+3 mg dexamethasone on day 5

Study Drugs and Dosage

Custirsen for infusion, 20 mg/mL is formulated as an isotonic, phosphate-buffered saline solution (pH 7.4) for iv administration and is supplied as an 8-mL solution containing 160 mg custirsen sodium in a single vial. Once prepared, the custirsen infusion is identical in appearance to the placebo infusion.

Once mixed for administration, the custirsen solution is stored at controlled room temperature and must be administered as close to the time of preparation as possible.

Using proper aseptic technique, two, three, or four 8-mL vials of 20 mg/mL custirsen (for a total of 320, 480, or 640 mg, respectively) is added to 0.9% sodium chloride (normal saline) up to a final volume of 250 mL, as close to the time of administration as possible.

The proper infusion pump rate is then set to allow for overfill in the normal saline bag and in the iv tubing to ensure the administration of the entire dose over the 2-hour infusion.

The line is flushed with sufficient 0.9% sodium chloride to ensure that all custirsen is flushed from the line and administered to the subject.

Vital Signs

Vital signs are measured at screening; check-in (day −2); day −1; days 1, 3, and 5 prior to the start of infusion and 6 and 12 hours after the start of infusion; days 2, 4, and 6 (24 hours after the start of infusion); day 7 prior to the start of infusion and 2, 4, and 6 hours after the start of infusion; day 8 (24 hours after the start of infusion); and at the follow-up visit for subjects in groups 1 and 2. Note: During the inpatient period vital signs are collected within ±30 minutes of the scheduled time point. Vital signs are also measured at other times if considered to be clinically appropriate.

Vital signs include the following:
pulse
blood pressure
respiratory rate
body temperature

Before pulse and blood pressure are measured, the subject must be in a supine position and resting for at least 10 minutes. (The same position and arm should be used each time vital signs are measured for a given subject.) For any abnormal vital sign finding, the measurement should be repeated as soon as possible. Any vital sign value that is judged by the investigator as a clinically significant change (worsening) from a baseline value is considered an adverse event.

### Physical Examinations

A full physical examination, including height (to be obtained at the screening visit only) and weight are performed at screening and on day 8 prior to discharge from the study center. Brief physical examinations are performed at check-in (day −2); baseline (day −1); days 1, 3, and 5 (prior to the start of infusion); and at the follow-up visit (for subjects in both groups 1 and 2). Any physical examination finding that is judged by the investigator as a clinically significant change (worsening) compared with a baseline value are considered an adverse event.

Results

Subjects administered a 320-mg dose of custirsen on day 1, a 480-mg dose of custirsen on day 3, and a 640-mg dose of the anti-clusterin oligonucleotide on day 5, are less likely to have a flu-like symptom compared to a corresponding human subject who is administered 640 mg of custirsen 3 times in the same 5 day period.

Subjects administered a 320-mg dose of custirsen on day 1, a 480-mg dose of custirsen on day 3, and a 640-mg dose of the anti-clusterin oligonucleotide on day 5 suffer from a flu-like symptom with reduced severity compared to a corresponding human subject who is administered 640 mg of custirsen 3 times in the same 5 day period.

The flu-like symptoms include fatigue, chills, diarrhea, constipation, nausea, vomiting, cough, insomnia, hot flush, headache, feeling cold, feeling hot, dizziness, upper abdominal pain, arthralgia, asthenia, back pain, chest discomfort, a headache, hyperhidrosis, myalgia, or pyrexia.

### Example 3

### Administration of Ascending Custirsen Doses (320, 480, and 640 mg) with Dexamethasone

Selection of Drugs and Dosages

The doses that are administered in this double-blind study (i.e., 320, 480, and 640 mg/day) are within the range of those being evaluated in Phase 3 studies.

Population to be Studied

Up to 165 healthy adult men enroll in this study. No women are included in the study.

Study Objectives

Objectives of the study are as follows:
to assess the pharmacokinetic profile of custirsen following administration of ascending custirsen doses (320, 480, and 640 mg)
to assess the safety and tolerability of custirsen in healthy men as assessed by the following throughout the study:
   adverse events and concomitant medication usage
   clinical laboratory tests
   vital signs measurements
   physical examination findings

General Design

This is a 3-arm, parallel-group, active- and placebo-controlled, double-blind, randomized study, to compare treatment with intravenous custirsen at 640 mg (highest intended therapeutic dose) with placebo as a negative control.

Following the baseline visit, subjects are randomly assigned to 1 of 3 treatment groups (custirsen—ascending dose [55 subjects], placebo for custirsen (55 subjects), custirsen—640 mg dose [55 subjects].

The 3 groups receive treatment as follows:
Group 1—Investigational Product (custirsen—ascending dose):
   320 mg custirsen+5 mg dexamethasone on day 1
   480 mg custirsen+5 mg dexamethasone on day 3
   640 mg custirsen+3 mg dexamethasone on day 5
Group 2—Placebo (normal saline):
   Placebo+5 mg dexamethasone on day 1
   Placebo+5 mg dexamethasone on day 3
   Placebo+3 mg dexamethasone on day 5
Group 3—Investigational Product (custirsen—640 mg Dose):
   640 mg custirsen+5 mg dexamethasone on day 1
   640 mg custirsen+5 mg dexamethasone on day 3
   640 mg custirsen+3 mg dexamethasone on day 5

Custirsen is administered iv using an infusion pump over a 2-hour period.

Placebo (commercially available normal saline) is administered iv using an infusion pump over a 2-hour period.

Dexamethasone is administered iv as a bolus 1 hour prior to custirsen administration.

Further details regarding study drug are provided in the “Study Drugs and Dosage” Section.

Study Drugs and Dosage

Custirsen for infusion, 20 mg/mL is formulated as an isotonic, phosphate-buffered saline solution (pH 7.4) for iv administration and is supplied as an 8-mL solution containing 160 mg custirsen sodium in a single vial. Once prepared, the custirsen infusion is identical in appearance to the placebo infusion. Custirsen is administered to subjects in group 1 as a single iv dose (320 mg on day 1, 480 mg on day 3, and 640 mg on day 5) using an infusion pump over a 2-hour period.

Once mixed for administration, the custirsen solution is stored at controlled room temperature and must be administered as close to the time of preparation as possible.

Using proper aseptic technique, two, three, or four 8-mL vials of 20 mg/mL custirsen (for a total of 320, 480, or 640 mg, respectively) is added to 0.9% sodium chloride (normal saline) up to a final volume of 250 mL, as close to the time of administration as possible.

The proper infusion pump rate is then set to allow for overfill in the normal saline bag and in the iv tubing to ensure the administration of the entire dose over the 2-hour infusion.

The line is flushed with sufficient 0.9% sodium chloride to ensure that all custirsen is flushed from the line and administered to the subject.

Other Study Drugs and Dosage

The placebo for custirsen is commercially available normal saline (provided by the investigational center). Placebo is administered iv using an infusion pump over a 2-hour period.

Dexamethasone is the commercially available Dexamethasone Sodium Phosphate Injection, United States Pharmacopeia (USP) (provided by the investigational center). A 5-mg dose of dexamethasone is administered iv as a bolus 1 hour prior to custirsen or placebo administration.

Assessment

The following pharmacokinetic parameters are calculated for custirsen using non-compartmental analyses if the data permits:
Cₘₐₓ
tₘₐₓ
AUC₀₋ₜ

Additional parameters may be calculated or analyses are conducted if deemed necessary.

Vital Signs

Vital signs are measured at screening; check-in (day −2); day −1; days 1, 3, and 5 prior to the start of infusion and 6 and 12 hours after the start of infusion; days 2, 4, and 6 (24 hours after the start of infusion); day 7; day 8; and at follow-up visits.

Vital signs include the following:
pulse
blood pressure
respiratory rate
body temperature

Before pulse and blood pressure are measured, the subject must be in a supine position and resting for at least 10 minutes. (The same position and arm should be used each time vital signs are measured for a given subject.) For any abnormal vital sign finding, the measurement should be repeated as soon as possible. Any vital sign value that is judged by the investigator as a clinically significant change (worsening) from a baseline value is considered an adverse event.

Physical Examinations

A full physical examination, including height (to be obtained at the screening visit only) and weight are performed at screening and on day 8 prior to discharge from the study center. Brief physical examinations are performed at check-in (day −2); baseline (day −1); days 1, 3, and 5 (prior to the start of infusion); and at the follow-up visit (for subjects in both groups 1-3). Any physical examination finding that is judged by the investigator as a clinically significant change (worsening) compared with a baseline value are considered an adverse event.

Results

Subjects administered a 320-mg dose of custirsen+5 mg dexamethasone on day 1, a 480-mg dose of custirsen+5 mg dexamethasone on day 3, and a 640-mg dose of the anti-clusterin oligonucleotide+5 mg dexamethasone on day 5 are less likely to have a flu-like symptom compared to a corresponding human subject who is administered 640 mg of custirsen 3 times in the same 5 day period.

Subjects administered a 320-mg dose of custirsen+5 mg dexamethasone on day 1, a 480-mg dose of custirsen+5 mg dexamethasone on day 3, and a 640-mg dose of the anti-clusterin oligonucleotide+5 mg dexamethasone on day 5, suffer from a flu-like symptom with reduced severity compared to a corresponding human subject who is administered 640 mg of custirsen 3 times in the same 5 day period.

The flu-like symptoms include fatigue, chills, diarrhea, constipation, nausea, vomiting, cough, insomnia, hot flush, feeling cold, feeling hot, dizziness, upper abdominal pain, arthralgia, asthenia, back pain, chest discomfort, headache, hyperhidrosis, myalgia, or pyrexia.

### Example 4

### Administration of Ascending Custirsen Doses (320, 480, and 640 mg) with Dexamethasone

Selection of Drugs and Dosages

The doses that are administered in this double-blind study (i.e., 320, 480, and 640 mg/day) are within the range of those being evaluated in Phase 3 studies.

Population to be Studied

Up to 165 healthy adult men enroll in this study. No women are included in the study.

Study Objectives

Objectives of the study are as follows:
to assess the pharmacokinetic profile of custirsen following administration of ascending custirsen doses (320, 480, and 640 mg)
to assess the safety and tolerability of custirsen in healthy men as assessed by the following throughout the study:
   adverse events and concomitant medication usage
   clinical laboratory tests
   vital signs measurements
   physical examination findings

General Design

This is a 3-arm, parallel-group, active- and placebo-controlled, double-blind, randomized study, to compare treatment with intravenous custirsen at 640 mg (highest intended therapeutic dose) with placebo as a negative control.

Following the baseline visit, subjects are randomly assigned to 1 of 3 treatment groups (custirsen—ascending dose [55 subjects], placebo for custirsen [55 subjects], custirsen—640 mg dose [55 subjects].

The 3 groups receive treatment as follows:
Group 1—Investigational Product (custirsen—ascending dose):
   320 mg custirsen+5 mg dexamethasone on day 1
   480 mg custirsen+5 mg dexamethasone on day 3
   640 mg custirsen+3 mg dexamethasone on day 5
   640 mg custirsen on day 7 under fasting conditions
Group 2—Placebo (normal saline):
   Placebo+5 mg dexamethasone on day 1
   Placebo+5 mg dexamethasone on day 3
   Placebo+3 mg dexamethasone on day 5
   Placebo on day 7 under fasting conditions
Group 3—Investigational Product (custirsen—640 mg Dose):
   640 mg custirsen+5 mg dexamethasone on day 1
   640 mg custirsen+5 mg dexamethasone on day 3
   640 mg custirsen+3 mg dexamethasone on day 5
   640 mg custirsen on day 7 under fasting conditions

Custirsen is administered iv using an infusion pump over a 2-hour period.

Placebo (commercially available normal saline) is administered iv using an infusion pump over a 2-hour period.

Dexamethasone is administered iv as a bolus 1 hour prior to custirsen administration.

Further details regarding study drug are provided in the “Study Drugs and Dosage” Section.

Study Drugs and Dosage

Custirsen for infusion, 20 mg/mL is formulated as an isotonic, phosphate-buffered saline solution (pH 7.4) for iv administration and is supplied as an 8-mL solution containing 160 mg custirsen sodium in a single vial. Once prepared, the custirsen infusion is identical in appearance to the placebo infusion. Custirsen is administered to subjects in group 1 as a single iv dose (320 mg on day 1, 480 mg on day 3, and 640 mg on days 5 and 7) using an infusion pump over a 2-hour period.

Once mixed for administration, the custirsen solution is stored at controlled room temperature and must be administered as close to the time of preparation as possible.

Using proper aseptic technique, two, three, or four 8-mL vials of 20 mg/mL custirsen (for a total of 320, 480, or 640 mg, respectively) is added to 0.9% sodium chloride (normal saline) up to a final volume of 250 mL, as close to the time of administration as possible.

The proper infusion pump rate is then set to allow for overfill in the normal saline bag and in the iv tubing to ensure the administration of the entire dose over the 2-hour infusion.

The line is flushed with sufficient 0.9% sodium chloride to ensure that all custirsen is flushed from the line and administered to the subject.

Other Study Drugs and Dosage

The placebo for custirsen is commercially available normal saline (provided by the investigational center). Placebo is administered iv using an infusion pump over a 2-hour period.

Dexamethasone is the commercially available Dexamethasone Sodium Phosphate Injection, United States Pharmacopeia (USP) (provided by the investigational center). A 5-mg dose of dexamethasone is administered iv as a bolus 1 hour prior to custirsen or placebo administration.

Assessment

The following pharmacokinetic parameters are calculated for custirsen using non-compartmental analyses if the data permits:
Cₘₐₓ
tₘₐₓ
AUC₀₋ₜ

Additional parameters may be calculated or analyses are conducted if deemed necessary.

Vital Signs

Vital signs are measured at screening; check-in (day −2); day −1; days 1, 3, and 5 prior to the start of infusion and 6 and 12 hours after the start of infusion; days 2, 4, and 6 (24 hours after the start of infusion); day 7 prior to the start of infusion and 2, 4, and 6 hours after the start of infusion; day 8 (24 hours after the start of infusion); and at the follow-up visit for subjects in groups 1-3. Note: During the inpatient period vital signs are collected within ±30 minutes of the scheduled time point. Vital signs are also measured at other times if considered to be clinically appropriate.

Vital signs include the following:
pulse
blood pressure
respiratory rate
body temperature

Before pulse and blood pressure are measured, the subject must be in a supine position and resting for at least 10 minutes. (The same position and arm should be used each time vital signs are measured for a given subject.) For any abnormal vital sign finding, the measurement should be repeated as soon as possible. Any vital sign value that is judged by the investigator as a clinically significant change (worsening) from a baseline value is considered an adverse event.

Physical Examinations

A full physical examination, including height (to be obtained at the screening visit only) and weight are performed at screening and on day 8 prior to discharge from the study center. Brief physical examinations are performed at check-in (day −2); baseline (day −1); days 1, 3, and 5 (prior to the start of infusion); and at the follow-up visit (for subjects in both groups 1-3). Any physical examination finding that is judged by the investigator as a clinically significant change (worsening) compared with a baseline value are considered an adverse event.

Results

Subjects administered a 320-mg dose of custirsen+5 mg dexamethasone on day 1, a 480-mg dose of custirsen+5 mg dexamethasone on day 3, a 640-mg dose of the anti-clusterin oligonucleotide+5 mg dexamethasone on day 5, and 640 mg custirsen on day 7 under fasting conditions are less likely to have a flu-like symptom compared to a corresponding human subject who is administered 640 mg of custirsen 4 times in the same 7 day period.

Subjects administered a 320-mg dose of custirsen+5 mg dexamethasone on day 1, a 480-mg dose of custirsen+5 mg dexamethasone on day 3, a 640-mg dose of the anti-clusterin oligonucleotide+5 mg dexamethasone on day 5, and 640 mg custirsen on day 7 under fasting conditions suffer from a flu-like symptom with reduced severity compared to a corresponding human subject who is administered 640 mg of custirsen 4 times in the same 7 day period.

The flu-like symptoms include fatigue, chills, diarrhea, constipation, nausea, vomiting, cough, insomnia, hot flush, feeling cold, feeling hot, dizziness, upper abdominal pain, arthralgia, asthenia, back pain, chest discomfort, headache, hyperhidrosis, myalgia, or pyrexia.

### Example 5

### Administration of Ascending Custirsen Doses (320, 480, and 640 mg) for Treatment of Lung Cancer

At least 50 lung cancer patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 6

### Administration of Ascending Custirsen Doses (320, 480, and 640 mg) for Treatment of Bladder Cancer

At least 50 bladder cancer patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 7

### Administration of Ascending Custirsen Doses (320, 480, and 640 mg) for Treatment of Ovarian Cancer

At least 50 ovarian cancer patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 8

### Administration of Ascending Custirsen Doses (320, 480, and 640 mg) for Treatment of NSCLC

At least 50 NSCLC patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 9

### Administration of Ascending Custirsen Doses (320, 480, and 640 mg) for Treatment of Renal Cancer

At least 50 renal cancer patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 10

### Administration of Ascending Custirsen Doses (320, 480, and 640 mg) for Treatment of Melanoma

At least 50 melanoma patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 11

### Administration of Ascending Custirsen Doses (320, 480, and 640 mg) for Treatment of Myeloma

At least 50 myeloma patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 12

### Administration of Ascending Custirsen Doses (320, 480, and 640 Mg) for Treatment of Breast Cancer

At least 50 breast cancer patients are separated into two groups.

Treatment of the first group begins with ascending doses of 320, 480, and 640 mg custirsen, followed by treatment with a combination of custirsen with a chemotherapeutic agent.

Treatment of the first group begins with ascending doses of 320, 480, and 640 mg custirsen in a 5 to 9 day period, followed by treatment with a combination of custirsen and a chemotherapeutic agent.

Treatment of the second group begins with three doses of 640 mg custirsen in a 5 to 9 day period, followed by treatment with a combination of custirsen and a chemotherapeutic agent.

The cancer condition of patients in each group improves in response to treatment. However, the first group has less fatigue, chills, diarrhea, constipation, nausea, vomiting, cough, insomnia, hot flush, dizziness, upper abdominal pain, arthralgia, asthenia, back pain, chest discomfort, headaches, hyperhidrosis, myalgia, or pyrexia compared to the second group.

### Example 13

### Selection of Drugs and Dosages

The doses that are administered in this double-blind study (i.e., 320, 480, and 640 mg/day) are within the range of those being evaluated in Phase 3 studies.

Population to be Studied

Up to 155 healthy adult men enroll in this study. The healthy subject population is selected based on the ICH E14 guidance for thorough QT studies. No women are included in the study.

Purpose of the Study and Study Objectives

Purpose of the Study

The purpose of this study is to assess the effect of custirsen on cardiac conduction and repolarization.

Study Objectives

The primary objective of this study is to assess the effect of a 640-mg dose of custirsen on cardiac conduction and repolarization as assessed by time-matched change from baseline in QTcI on day 7. The secondary objectives of the study are as follows:
to assess the pharmacokinetic profile of custirsen following administration of ascending custirsen doses (320, 480, and 640 mg)
to assess the relationship of the QT QTc interval with plasma concentrations of custirsen
to assess the safety and tolerability of custirsen in healthy men as assessed by the following throughout the study:
   adverse events and concomitant medication usage
   clinical laboratory tests
   vital signs measurements
   physical examination findings
   12-lead ECG
to demonstrate assay sensitivity using moxifloxacin (400 mg) as a positive control

Study Design

General Design

This is a 3-arm, parallel-group, active- and placebo-controlled, double-blind, randomized study, to compare treatment with intravenous custirsen at 640 mg (highest intended therapeutic dose) with placebo as a negative control. A group receiving a single oral dose of 400 mg moxifloxacin (day 7) is included in the study as a positive control to demonstrate assay sensitivity.

This study was designed in compliance with the final version of the ICH E14 guidance. The key elements of which include the following: (1) the use of a positive control (moxifloxacin), (2) robust characterization of the test drug response relationship at the maximum therapeutic exposure, (3) potential methods for reducing variability in the measurement of the QTc interval using QTc hysteresis control methods, replicate ECGs and a centralized core laboratory to measure QTc interval utilizing appropriate digital technology with a full cardiologist over read, and (4) the statistical power to exclude as much as 10 msec QT/QTc prolongation using the upper arm of the 95% one-sided confidence interval (CI) (of the placebo-corrected change in QTc interval from baseline) as the regulatory threshold for declaring the study negative (i.e., no evidence for relevant QT/QTc prolongation).

On days 1, 3, 5, and 7, subjects are administered their respective treatments as follows:

The 3 groups receive treatment as follows:
group 1—investigational product (custirsen):
   320 mg custirsen+5 mg dexamethasone on day 1
   480 mg custirsen+5 mg dexamethasone on day 3
   640 mg custirsen+3 mg dexamethasone on day 5
   640 mg custirsen on day 7 under fasting conditions
group 2—placebo (normal saline):
   placebo+5 mg dexamethasone on day 1
   placebo+5 mg dexamethasone on day 3
placebo+3 mg dexamethasone on day 5
   placebo on day 7 under fasting conditions
group 3—positive control (Moxifloxacin):
   placebo+5 mg dexamethasone on day 1
   placebo+5 mg dexamethasone on day 3
   placebo+3 mg dexamethasone on day 5
   moxifloxacin 400 mg+placebo (immediately after moxifloxacin administration) on day 7 under fasting conditions

Primary and Secondary Measures and Endpoints

Primary Pharmacodynamic Measure and Endpoints

The primary ECG variable and endpoint for this study is the time-matched change from baseline in QTcI method on day 7 at the following time points: 1, 2 (end of infusion), 2.5, 3, 4, 5, 6, 8, 12, 16, 20, and 23.5 hours.

Secondary Pharmacodynamic Measures and Endpoints

Secondary variables and endpoints for this study are as follows:
QTcF time-matched change from baseline on day 7 at the following time points: 1, 2 (end of infusion), 2.5, 3, 4, 5, 6, 8, 12, 16, 20, and 23.5 hours
heart rate, PR interval, QRS interval and uncorrected QT interval time-matched change from baseline on day 7
exploratory outlier analyses on day 7 (absolute values and changes from baseline)
change in ECG morphological patterns on day 7, as compared to baseline
relationship between the placebo-corrected QTc (QTcI and QTcF) change from baseline and plasma concentrations of custirsen (pharmacokinetic/pharmacodynamic analysis)
a comparison between the active control, moxifloxacin (400 mg), and placebo are also performed to demonstrate assay sensitivity as required by current regulatory guidance

Pharmacokinetic Measures and Endpoints

The following pharmacokinetic parameters are calculated for custirsen using non-compartmental analyses:
maximum observed plasma concentration (Cₘₐₓ)
time to maximum observed plasma concentration (tₘₐₓ)
area under the curve to the time of last measurable concentration (AUC₀₋ₜ)

Additional parameters and/or additional metabolite profiles are calculated if deemed necessary.

Study Drugs and Dosage

Investigational Product and Dosage

Custirsen for infusion, 20 mg/mL is formulated as an isotonic, phosphate-buffered saline solution (pH 7.4) for iv administration and is supplied as an 8-mL solution containing 160 mg custirsen sodium in a single vial. Once prepared, the custirsen infusion is identical in appearance to the placebo infusion. Custirsen is administered to subjects in group 1 as a single iv dose (320 mg on day 1, 480 mg on day 3, and 640 mg on days 5 and 7) using an infusion pump over a 2-hour period.

Once mixed for administration, the custirsen solution is stored at controlled room temperature and must be administered as close to the time of preparation as possible.

Using proper aseptic technique, two, three, or four 8-mL vials of 20 mg/mL custirsen (for a total of 320, 480, or 640 mg, respectively) is added to 0.9% sodium chloride (normal saline) up to a final volume of 250 mL, as close to the time of administration as possible.

The proper infusion pump rate is then set to allow for overfill in the normal saline bag and in the iv tubing to ensure the administration of the entire dose over the 2-hour infusion.

The line is flushed with sufficient 0.9% sodium chloride to ensure that all custirsen is flushed from the line and administered to the subject.

Other Study Drugs and Dosage

The placebo for custirsen is commercially available normal saline (provided by the investigational center). Placebo is administered iv using an infusion pump over a 2-hour period.

Dexamethasone is the commercially available Dexamethasone Sodium Phosphate Injection, United States Pharmacopeia (USP) (provided by the investigational center). A 5-mg dose of dexamethasone is administered iv as a bolus 1 hour prior to custirsen or placebo administration. Moxifloxacin is the commercially available moxifloxacin (400-mg tablets) (provided by the investigational center). Moxifloxacin is administered orally.

Study Procedures

Study procedures and assessments with their timing are summarized in Table 1.

TABLE 1
Study Procedures and Assessments

| | Pretreatment | | | | | | | | | | | | | Follow- |
|---|---|---|---|---|---|---|---|---|---|---|---|---|---|---|
| | V1 | | Double-blind treatment period | | | | | | | | | | | up^{a} |
| | Screening | V2 | V2 | | | | | | | | | V3^{a} | V4^{a} | V5 |
| | (days −28 | Check in | Day | Day | Day | Day | Day | Day | Day | Day | Day | Day | Day | Day 14 |
| Procedures and assessments | to −3) | (day −2) | −1 | 1 | 2 | 3 | 4 | 5 | 6 | 7 | 8 | 9 | 10 | (±2 days) |
| Informed consent | X | | | | | | | | | | | | | |
| Medical history | X | X^{b} | | | | | | | | | | | | |
| Prior medication history | X | X^{b} | | | | | | | | | | | | |
| Inclusion and exclusion criteria | X | X^{b} | | | | | | | | | | | | |
| Urine drug and cotinine screen | X | X | | | | | | | | | | | | |
| Alcohol screen | | X | | | | | | | | | | | | |
| Randomization | | | X | | | | | | | | | | | |
| Study restriction and | X | X | | | | | | | | | | | | |
| compliance review | | | | | | | | | | | | | | |
| Physical examination^{c} | X | X | | X | | X | | X | | X | X | | | X |
| Vital signs measurement^{d} | X | X | X | X | X | X | X | X | X | X | X | | | X |
| Clinical laboratory tests^{e} | X | X | | X | X | X | X | X | X | X | X | | | X |
| Urinalysis^{f} | X | X | | | | | | | | | X | | | |
| Serology and TB T-spot test | X | | | | | | | | | | | | | |
| Safety 12-lead ECG^{g} | X | X | | X | | X | | X | | X | X | | | X |
| Holter ECG^{H} | | | X | | | | | | | Xⁱ | | | | |
| Custirsen (or placebo) | | | | X | | X | | X | | X | | | | |
| administration^{j} | | | | | | | | | | | | | | |
| Dexamethasone administration^{k} | | | | X | | X | | X | | | | | | |
| Moxifloxacin administration^{l} | | | | | | | | | | X | | | | |
| Blood samples for | | | X | X | | X | | X | | X | X | X | X | X |
| pharmacokinetics^{m} | | | | | | | | | | | | | | |
| Adverse event inquiry | | X | X | X | X | X | X | X | X | X | X | | | X |
| Concomitant medication inquiry | | X | X | X | X | X | X | X | X | X | X | | | X |

^{a}Groups 1 and 2 only.
^{b}A review and update of these items are performed at check-in to ensure continued subject eligibility.
^{c}A full physical examination are performed at screening and on day 8 prior to discharge. An abbreviated physical examination are performed at admission and on day −1, on days 1, 3, and 5 (predose), and at the follow-up visit for subjects in groups 1 and 2. On day 7, the physical examination is performed prior to the start of infusion (0 hours) and at the end of infusion (2 hours).
^{d}Blood pressure, pulse, respiratory rate, and temperature (after at least 10 minutes of supine rest) is measured at screening, check-in (day −2), and on day −1. On days 1, 3, and 5 (predose), vital signs are measured 6, 12, and 24 hours postdose). On day 7, vital signs are measured prior to study drug administration (0 hour) and 2, 4, 6, and 24 hours after study drug administration. For subjects in groups 1 and 2, vital signs are also measured at the follow-up visit.
^{e}Clinical laboratory tests (serum chemistry and hematology) are performed at screening, check in (day −2), and day −1. On days 1, 3, and 5 (predose and 24 hours after study drug administration). On day 7, clinical laboratory tests are performed prior to the start of infusion (0 hour), at the end of infusion (2 hours), and 6 and 24 hours after study drug administration. For subjects in groups 1 and 2, clinical laboratory tests are also performed at the follow-up visit.
^{f}Urinalysis are performed at screening, check-in (day −2), and prior to discharge on day 8.
^{g}Standard 12-lead ECGs are recorded at screening; check-in (day −2); day −1, on days 1, 3, and 5 (predose); and at the follow-up visit. On day 7, an ECG is recorded prior to study drug administration (0 hour) and 2 and 24 hours after study drug adminstration.
^{h}Holter 12-lead ECGs are collected on day −1 at 0, 1, 2, 2.5, 3, 4, 5, 6, 8, 12, 16, 20, and 23.5 hours after hypothetical custirsen administration.
ⁱHolter 12-lead ECGs are collected on day 7 predose and 1, 2, 2.5, 3, 4, 5, 6, 8, 12, 16, 20, and 23.5 hours after the start of custirsen infusion.
^{j}Group 1 receives a 320-mg dose of custirsen on day 1, a 480-mg dose of custirsen on day 3, and a 640-mg dose of custirsen on days 5 and 7. Subjects in group 2 receive placebo for custirsen on the same days.
^{k}A 5-mg dose of dexamethasone are administered as a pre-medication on days 1 and 3, 1 hour prior to the start of custirsen infusion. A 3-mg dose of dexamethasone are administered on day 5 one hour prior to the start of the custirsen infusion.
^{l}Subjects in group 3 receives a 400-mg dose of moxifloxacin on day 7, immediately prior to the start of placebo for custirsen infusion.
^{m}Blood samples (3-mL each) for pharmacokinetic analysis is taken from all subjects on day −1 within 90 minutes before the start of hypothetical custirsen infusion. On days 1, 3, and 5, samples are taken within 15 minutes prior to the start of infusion and at the end of infusion (2 hours). On day 7, samples are taken within 15 mintues prior to infusion, and 1, 2, (end of infusion), 2.5, 3, 4, 5, 6, 8, 12, 16, 20, and 23.5 hours after the start of infusion. Subjects in groups 1 and 2 return for additional pharmacokinetic sampling on days 9 and 10 (48 and 72 hours after the start of infusion on day 7, respectively). Additional pharmacokinetic samples are taken on day 14 for subjects in groups 1 and 2 (168 hours after the start of infusion on day 7). A 1-hour window is permitted for pharmacokinetic samples drawn 23.5 hours after infusion. A 2-hour window is permitted for pharmacokinetic samples taken from 48 through 72 hours after infusion, and a 2-day window is permitted for pharmacokinetic samples taken 168 hours after infusion.

Treatment of Subjects

Study Drugs Administered

Following the baseline visit, subjects are randomly assigned to 1 of 3 treatment groups (custirsen [55 subjects], placebo for custirsen [55 subjects], or moxifloxacin [45 subjects],

The 3 groups receive treatment as follows:
Group 1—Investigational Product (custirsen):
   320 mg custirsen+5 mg dexamethasone on day 1
   480 mg custirsen+5 mg dexamethasone on day 3
   640 mg custirsen+3 mg dexamethasone on day 5
   640 mg custirsen on day 7 under fasting conditions
Group 2—Placebo (normal saline):
   Placebo+5 mg dexamethasone on day 1
   Placebo+5 mg dexamethasone on day 3
   Placebo+3 mg dexamethasone on day 5
   Placebo on Day 7 under fasting conditions
Group 3—Positive control (moxifloxacin):
   Placebo+5 mg dexamethasone on day 1
   Placebo+5 mg dexamethasone on day 3
   Placebo+3 mg dexamethasone on day 5
   400 mg Moxifloxacin+placebo (immediately after moxifloxacin administration) on day 7 under fasting conditions

Custirsen is administered iv using an infusion pump over a 2-hour period.

Placebo (commercially available normal saline) is administered iv using an infusion pump over a 2-hour period.

Dexamethasone is administered iv as a bolus 1 hour prior to custirsen administration.

Moxifloxacin (400 mg) is administered orally.

Further details regarding study drug are provided in the “Study Drugs and Dosage” Section.

Assessment of Pharmacodynamics and Pharmacokinetics

Pharmacodynamics

Pharmacodynamic Variables

Holter ECGs is performed at baseline (day −1) and prior to the start of infusion on day 7 and 1, 2 (end of infusion), 2.5, 3, 4, 5, 6, 8, 12, 16, 20, and 23.5 hours after the start of infusion.

The primary ECG variable and endpoint for this study is the time-matched change from baseline in QTcI method on day 7 at each time point.

Secondary variables and endpoints for this study are as follows:
QTcF time-matched change from baseline on day 7 at the following time points: 1, 2 (end of infusion), 2.5, 3, 4, 5, 6, 8, 12, 16, 20, and 23.5 hours.
heart rate, PR interval, QRS interval and uncorrected QT interval time-matched change from baseline on day 7
exploratory outlier analyses on day 7 (absolute values and changes from baseline)
change in ECG morphological patterns on day 7, as compared to baseline
relationship between the placebo-corrected QTc (QTcI and QTcF) change from baseline and plasma concentrations of custirsen (pharmacokinetic/pharmacodynamic analysis)
a comparison between the active control, moxifloxacin (400 mg), and placebo is also performed to demonstrate assay sensitivity as required by current regulatory guidance

Pharmacodynamic Assessment

Electrocardiograms are recorded on a 1000 Hz flash card. ECGs to be used in the analyses are selected at predetermined time points as detailed below and are read centrally using a semiautomatic, high-resolution, on-screen caliper method with annotations. The central ECG laboratory are blinded to subject identifiers, treatment, and visit. All ECGs for a given subject are analyzed by a single ECG analyzer.

All 24-hour 12-lead ECGs are downloaded on days −1 and 7 from the flash cards. Endpoint 12-ECGs are downloaded in triplicate within approximately 10 minutes of the following nominal time points on days—1 (baseline profile day) and 7 (time-matched): 0 (pre-dose) and 1, 2 (end of infusion), 2.5, 3, 4, 5, 6, 8, 12, 16, 20, and 23.5 hours (13 time points).

The time of Holter startup on days −1 and 7 are just prior to the scheduled dose of custirsen or placebo administration (or hypothetical administration time). For subjects in group 3 on day 7, the time of Holter startup is just prior to the scheduled moxifloxacin dose administration time.

Subjects must be resting, in a comfortable supine position and environment, for at least 15 minutes before each ECG sampling time point through to at least 5 minutes after the scheduled time point.

The primary lead for interval measurements is Lead II. However, if technical issues or unstable heart rate exist, the secondary lead is V5. A tertiary lead may be used if severe technical issues or unstable heart rate exist with the primary or secondary leads. The same lead should be used for interval measurements between baseline and treatment data.

If targeted ECG time points are artifactual and of poor quality, the central ECG lab captures analyzable 10-second ECGs as close as possible to the targeted time points. Each digital ECG contains no less than 3 analyzable heart beats (RR intervals).

Each fiducial point (e.g., onset of QRS, offset of T wave) is electronically marked. The original ECG waveform and such annotations are saved separately in XML format for independent review.

When ECGs coincide with blood sample collections, vital sign assessments, or meals, the Holter ECGs are performed first, followed by safety ECGs and vital signs. After that the blood for pharmacokinetic samples are collected as close to the scheduled time point as possible, immediately followed by the safety samples for clinical laboratory, and meals are the last item.

The primary QT to QTc correction formula is an Individual QT correction (QTcI). The following corrected QT is also computed:

Fredericia's correction:QTcF=QT/RR^{1/3}

The Bazett correction formula is also used for data analysis of results. The formula is:

Bazett's correction:QTcB=QT/RR1/2

The mean values of heart rate, QT, PR, and QRS intervals for the triplicate ECGs at each time point are calculated and used as the ECG parameters for the relevant time point. QTc values for each time point are derived using the relevant mean RR and QT values.

Pharmacokinetics

Pharmacokinetic Variables

The following pharmacokinetic parameters are calculated for custirsen using non-compartmental analyses if the data permits:
Cₘₐₓ
tₘₐₓ
AUC₀₋ₜ

Additional parameters may be calculated or analyses are conducted if deemed necessary.

Vital Signs

Vital signs are measured at screening; check-in (day −2); day −1; days 1, 3, and 5 prior to the start of infusion and 6 and 12 hours after the start of infusion; days 2, 4, and 6 (24 hours after the start of infusion); day 7 prior to the start of infusion and 2, 4, and 6 hours after the start of infusion; day 8 (24 hours after the start of infusion); and at the follow-up visit for subjects in groups 1 and 2. Note: During the inpatient period vital signs are collected within ±30 minutes of the scheduled time point. Vital signs are also measured at other times if considered to be clinically appropriate.

Vital signs include the following:
pulse
blood pressure
respiratory rate
body temperature

Before pulse and blood pressure are measured, the subject must be in a supine position and resting for at least 10 minutes. (The same position and arm should be used each time vital signs are measured for a given subject.) For any abnormal vital sign finding, the measurement should be repeated as soon as possible. Any vital sign value that is judged by the investigator as a clinically significant change (worsening) from a baseline value is considered an adverse event.

Electrocardiography

The standard digital 12-lead ECGs for bedside safety assessment is recorded using the study center's own ECG equipment. A standard 12-lead resting ECG are recorded after the subject had been in a supine position for at least 10 minutes.

A 12-lead ECG is conducted at screening; check-in (day −2); baseline (day −1); days 1, 3, and 5 (prior to the start of infusion); day 7 (prior to the start of infusion and at the end of infusion [2 hours]); day 8 (24 hours after the start of infusion on day 7); and at the follow-up visit (for subjects in groups 1 and 2). Safety ECGs may be performed at other times if considered to be clinically appropriate by the investigator. The bedside safety ECGs are not centrally read or used for data analyses. They are evaluated by the investigator or a qualified designee for bedside safety monitoring. A paper copy of each safety ECG is kept in the study folder. Any ECG finding that is judged by the investigator as a clinically significant change (worsening) compared with a baseline value is considered an adverse event.

Note: ECG interval and morphology changes that are recorded at the screening visit are used only for the consideration of the exclusion criteria and re not included in the formal analyses.

Physical Examinations

A full physical examination, including height (to be obtained at the screening visit only) and weight are performed at screening and on day 8 prior to discharge from the study center. Brief physical examinations are performed at check-in (day −2); baseline (day −1); days 1, 3, and 5 (prior to the start of infusion); and at the follow-up visit (for subjects in groups 1 and 2). Any physical examination finding that is judged by the investigator as a clinically significant change (worsening) compared with a baseline value are considered an adverse event.

Pharmacodynamic Analysis

Primary Variable

The primary ECG variable and endpoint for this study is the time-matched change from baseline in QTcI method on day 7 at the following time points: 1, 2 (end of infusion), 2.5, 3, 4, 5, 6, 8, 12, 16, 20, and 23.5 hours.

Secondary Variables

Secondary variables and endpoints for this study are as follows;
QTcF time-matched change from baseline on day 7 at the following time points: 1, 2 (end of infusion), 2.5, 3, 4, 5, 6, 8, 12, 16, 20, and 23.5 hours.
heart rate, PR interval, QRS interval and uncorrected QT interval time-matched change from baseline on day 7
exploratory outlier analyses at day 7 (absolute values and changes from baseline)
change in ECG morphological patterns at day 7, as compared to baseline
relationship between the placebo-corrected QTc (QTcI and QTcF) change from baseline and plasma concentrations of custirsen (pharmacokinetic/pharmacodynamic analysis)
a comparison between the active control, moxifloxacin (400 mg), and placebo is also performed to demonstrate assay sensitivity as required by current regulatory guidance

Planned Method of Analysis

QT Correction Formulae

The physiologically inversed relationship between heart rate and QT interval requires an adjustment process to correct, or normalize, the QT interval to the heart rate. Therefore, the corrected QT interval (QTc) allows comparisons of QTc intervals across a range of heart rates.

The primary QT to QTc correction formula is determined for each subject by iterating the QT-RR relationship using the baseline ECGs (day −1) in order to find an estimate for the exponent such that the slope of this relationship is closest to 0.

QTcI is the individually determined QT correction, and the goal is to find β such that QTcI is a constant, where QTcI=QT/(RR)^{β}. This implies log(QTcI)=log(QT)−β×log (RR). Because log(QTcI) is a constant, one can re-write this equation as log(QT)=α+β×log(RR). Therefore, the exponent estimate can be obtained by numerical iteration such that slope for QT-RR relationship is closest to 0 or using regression analysis on log-transformed data based on the least squares approach.

The following corrected QT is also computed:

Fridericia's correction:QTcF=QT/RR^{1/3}

The mean values of heart rate, QT, PR, and QRS for the 3 replicate ECGs at each time point are calculated and used as the ECG parameters for the relevant time point. QTc values for each time point are derived using the relevant mean RR and QT values.

ECG Data Points for Primary Analysis

ECG data is obtained for all treatment groups at the following times:
day −1 (baseline profile day): 1, 2 (end of infusion), 2.5, 3, 4, 5, 6, 8, 12, 16, 20, and 23.5 hours
day 7 (time-matched): 1, 2 (end of infusion), 2.5, 3, 4, 5, 6, 8, 12, 16, 20, and 23.5 hours

Primary Pharmacodynamic Analysis

The primary analyses for the QT/QTc data in this study are based on the time-matched analyses for day 7. These time-matched analyses are based upon the endpoint change from baseline in QTc (QTcI) interval. For each individual subject, the baseline value is subtracted from the time-matched value on day 7. Note that the multiple ECGs around a time point are averaged to produce a single value for each ECG interval for that time point.

To evaluate the drug effect, the statistical hypotheses can be stated as follows:

H₀:∪{μ_custirsen(i)−μ_placebo(i)}≧10 msec,i=1,2, . . . ,K

and

H_{A}:∩{μ_custirsen(i)−μ_placebo(i)}<10 msec,i=1,2, . . . ,K

where μ_custirsen(i) and μ_placebo(i) are the mean change from baseline of QTc for the drug and placebo at time point i for K time points, respectively. The Intersection-Union test can be applied here; therefore, no multiple endpoint adjustment is needed. Based on E14 Guidance, the null hypothesis is rejected if all of the time points have a 1-sided upper 95% confidence bound (i.e., equivalent to the upper bound of a 2-sided 90% confidence bound) less than 10 msec.

The statistical model is a Repeated Measures model (SAS® MIXED procedure with REPEATED sub-command). The model includes the following fixed effects: time (categorical), treatment arm and time by treatment interaction. The compound symmetry (CS) covariance matrix for repeated observations within subjects is used, unless a different covariance structure (among the following: Autoregressive (1), Heterogeneous Autoregressive (1) Heterogeneous Compound Symmetry or Unstructured) provides a better fit as evident by the best Akaike information criterion (AIC).

Analyses of individual timepoints separately are included as supportive.

If only 1 time point of the time points marginally exceeds 10 msec, an assessment whether this is a false positive finding relies on findings from the concentration -QTc (pharmacokinetic/pharmacodynamic) analysis described below.

The analyses is also presented in a graphical manner as follows: for each comparison of interest all confidence intervals (CIs) (corresponding to the post-baseline time points) are presented superimposed in 1 graph showing custirsen and moxifloxacin, both having been placebo corrected. Whereas QTcI is primary, QTc analyses are separately done for QTcI and QTcF.

Assay Sensitivity Analysis

To establish assay sensitivity, at least 1 time point where the mean difference of moxifloxacin and placebo is statistically significantly greater than 5 msec are shown. This can be accomplished by setting up the following statistical hypotheses:

H₀:∩{μ_moxifloxacin(i)−μ_placebo(i)}≦5 msec,i=1,2, . . . ,L

and

H_{A}:∪{μ_moxifloxacin(i)−μ_placebo(i)}>5 msec,i=1,2, . . . ,L

where μ_moxifloxacin(i) and μ_placebo(i) represent the mean value of a time-matched change from baseline of QTcI for moxifloxacin and the placebo at time point i, respectively. L is the number of time points selected to evaluate moxifloxacin effect.

The null hypothesis is rejected if the lower bound of the 1-sided (corrected for multiplicity) 95% CI is above 5 msec for 1 time point or more. Detecting the effect of the positive control establishes the ability of the study to detect such an effect of the study drugs.

For purposes of determining assay sensitivity, 4 time points (i.e., hours 2, 3, 4 and 5) are utilized for calculating the confidence intervals. In this case, since the alternative hypothesis is that at least 1 of the time points is greater than 5 msec, the multiplicity adjustment is necessary. Therefore, the confidence intervals are calculated using Hochberg step-up adjustment. For that purpose, the time points are ordered in an increasing order according to their lower bound of the 1-sided 95% CI. Adjusted confidence intervals are calculated based on the following Hochberg adjusted 1-sided alpha error level: 0.05, 0.025, 0.0167, and 0.0125, corresponding to the ordered time points.

Although these 4 time points are used to assess assay sensitivity, the entire moxifloxacin profile is displayed.

Descriptive Analyses

Descriptive statistics are presented for all ECG parameters for each treatment by time point. Descriptive statistics are also presented per treatment on the change from baseline for all ECG parameters. The time-matched baseline is used for this purpose.

Outlier Analysis

An exploratory outlier or categorical analysis supplements the central tendency analyses (exploratory) by determining if there were subjects who had an exaggerated effect on any ECG interval that would not be revealed in a mean change from baseline central tendency analyses. Each subject would be evaluated for an outlier value based on the most extreme value across all of the post-baseline time points.

The following criteria (study endpoints) are defined for this analysis:
For QTc: A value for a subject is considered to be an outlier at a post-baseline time point if the QTc interval at that follow-up time point is >500 msec and the subject's baseline mean QTc interval is ≦500 msec. Outlier values are also presented for the >480 cut-point and for the 450 cut-point. In addition, subjects with changes from baseline of >30-60 msec and >60 msec are identified.
For QT parameter: A value for a subject is considered to be an outlier at a post-baseline time point if the QT interval at that follow-up time point is >500 msec and the subject's baseline mean QT interval is ≦500 msec.
For PR: A value for a subject is considered to be an outlier at a post-baseline time point if the PR interval at that follow-up time point is >200 msec and it is at least a 25% increase from the subject's baseline mean PR interval.
For QRS: A value for a subject is considered to be an outlier at a pre-determined post-baseline time point if the QRS interval at that follow-up time point is >100 msec and it is at least a 25% increase from the subject's baseline mean QRS interval.
For heart rate: A value for a subject is considered to be an outlier at a post-baseline time point if the heart rate measurement at that follow-up time point is <45 bpm and the measure is at least a 25% decrease from the subject's baseline mean (i.e., a bradycardic event) or if the heart rate is >100 bpm and the measure is at least a 25% increase from the baseline mean heart rate (i.e., a tachycardic event).

The number and percentage of subjects meeting each of the above criteria is presented for the treatment and control groups.

Morphological Analysis

New onset (defined as being not present on any baseline ECG but present on any on treatment ECG) is analyzed for the following variables:
second degree heart block
third degree heart block
atrial fibrillation
atrial flutter
complete right bundle branch block
complete left bundle branch block
ST segment change (elevation and depression separately)
T-wave abnormalities (negative T waves only)
myocardial infarction pattern
new abnormal U waves
any other clinically significant morphological or conduction abnormality

The number and percentage of subjects meeting new onset morphological abnormality is presented for the treatment and control groups.

Multiple Comparisons and Multiplicity

As noted in the “Primary Pharmacodynamic Analysis” Section, no multiple endpoint adjustment is needed for the comparison of custirsen with placebo. For the comparison of moxifloxacin with placebo (assay sensitivity analysis), the Hochberg step-up adjustment is used to adjust for the multiple time points evaluated in the analysis (“Assay Sensitivity Analysis” Section).

Pharmacokinetic Analysis

Pharmacokinetic parameters are summarized using descriptive statistics.

Pharmacokinetic/Pharmacodynamic Analysis

A pharmacokinetic/pharmacodynamic analysis is performed, using only the subjects who receive custirsen.

A linear mixed effects modeling approach is used to quantify the relationship between the plasma concentration of custirsen and ΔΔQTcI (time-matched drug-placebo difference in QTc interval, baseline-adjusted, the delta-delta approach). Then, the predicted population average expected ΔΔQTc and corresponding upper 95% 1-sided CI at relevant concentration levels, e.g., the mean Cₘₐₓ under therapeutic dose, is estimated. Whereas QTcI is primary variable for this analysis, QTcF is analyzed as well as supportive.

Exploration of the adequacy of the model fit to the assumption of linearity and the impact on quantifying the concentration response relationship is performed. Additional exploratory analyses (via graphical displays and/or model fitting) may include investigation of a delayed effect and justification for the choice of pharmacodynamic model (linear versus nonlinear). Additional analyses may be performed on additional metabolites. In addition, alternative dependent variable such as QTc or ΔQTc is considered.

Results

Subjects administered a 320-mg dose of custirsen+5 mg dexamethasone on day 1, a 480-mg dose of custirsen+5 mg dexamethasone on day 3, a 640-mg dose of the anti-clusterin oligonucleotide+5 mg dexamethasone on day 5, and 640 mg custirsen on day 7 under fasting conditions are less likely to have a flu-like symptom compared to a corresponding human subject who is administered 640 mg of custirsen 4 times in the same 7 day period.

Subjects administered a 320-mg dose of custirsen+5 mg dexamethasone on day 1, a 480-mg dose of custirsen+5 mg dexamethasone on day 3, a 640-mg dose of the anti-clusterin oligonucleotide+5 mg dexamethasone on day 5, and 640 mg custirsen on day 7 under fasting conditions suffer from a flu-like symptom with reduced severity compared to a corresponding human subject who is administered 640 mg of custirsen 4 times in the same 7 day period.

The flu-like symptoms include fatigue, chills, diarrhea, constipation, nausea, vomiting, cough, insomnia, hot flush, feeling cold, feeling hot, dizziness, upper abdominal pain, arthralgia, asthenia, back pain, chest discomfort, headache, hyperhidrosis, myalgia, or pyrexia.

### Example 14

### In Vivo Activity of Custirsen Against HMI 8226 (Human Myeloma) Xenograft Model, Implanted Subcutaneously into Athymic Nude Mice

Summary

This study aimed to evaluate the effect of Custirsen (OGX-11, TV-1011) in combination with Velcade® (Bortezomib) against human myeloma model in nude mice. The cells were implanted subcutaneously into female SCID mice with 7×10⁶ cells inoculum. On day 20, when the tumors reached 120-170 mm³, mice were sorted into five treatment groups (n=10): control group; Custirsen (OGX) 40 mg/kg ip qd*5, then twk; Velcade® 0.5 mg/kg iv biwk; custirsen 20 mg/kg ip qd*5, twk+Velcade® 0.5 mg/kg iv biwk; custirsen 40 mg/kg ip qd*5, twk+Velcade® 0.5 mg/kg iv biwk.

Tumors and body weights were measured weekly until termination of the study on day 71. Response to treatment was evaluated for tumor growth inhibition (TGI) and tumor growth delay (TGD).

Treatment with Velcade® 0.5 mg/kg had moderate but not statistically significant efficacy.

Treatment with custirsen at a dose of 40 mg/kg was stopped after 7 injections due to toxicity. Nevertheless, either alone or in combination, it significantly inhibited tumor growth by the end of the study.

Introduction

The objective of this study was to evaluate the effect of custirsen (OGX) in combination with Velcade® (Bortezomib) against human myeloma model in nude mice.

Materials and Methods

a. Materials
RPMI 8226 (Human Plasmacytoma, Myeloma B Cells) ATCC # CCL-155;
RPMI medium (Beit Haemek);
ECM Gel (Mtrigel), Sigma-Aldrich, Cat # E1270, 5 ml;
HBSS (Belt Haemek,);
Velcade® (Bortezomib) 3.5 mg lyophilized in vial, Mnf. 09-2011;
Custirsen (OGX-11, TV-1011) 20 mg/ml, K-46138;
Sodium chloride, TEVA.

b. Animals

70 CB.17 SCID female mice, 4-6 weeks old, 16-20 grams, obtained from Harlan animal breeding center.

c. Cell Preparation

Harvested 14 flasks (T-175), passage 5, which were split 1:4. Sample from the cell suspension was taken for counting (0.3 ml in duplicates for CEDEX) before spun down. Cell viability was 85.6% and live cell concentration was 167×10⁵ cells/ml. The pellet was resuspended in HBSS to a final volume of 8 ml.

d. Study Design

Tumors were implanted subcutaneously with RPMI-8226 cells into the right flank of the mouse on Day 0. Each animal received a s.c. injection 7×10⁶ cells in 0.1 ml suspension. On day 20, mice were sorted by the optimal tumor volume (120-170=0) and were allocated into 5 groups of 10 mice. Mice were individually tagged and their tumor volume and body weight were monitored weekly during the study. Tumor size was measured by caliper and calculated using the formula:

$$π \times {( \frac{width}{2} )}^{2} \times length .$$

The treatment started on day 11 (0.2 ml per 20 grams v/w) and continued till day 52, after which the remaining mice were left for observation until day 71. Treatment regimen and doses are indicated in Table 2.

TABLE 2
Experimental design

| | | Drug and treatment | | | |
|---|---|---|---|---|---|
| Gr. | N | Agent | mg/kg | Route | Schedule |
| 1 | 10 | Vehicle | — | ip | ip qd*5 + twk |
| 2 | 10 | Custirsen (OGX) | 40 | ip | ip qd*5 + twk |
| 3 | 10 | Velcade ® | 0.5 | iv | biwk |
| 4 | 10 | Velcade ® + | 0.5 | iv | biwk |
| | | Custirsen (OGX) | 20 | ip | ip qd*5 + twk |
| 5 | 10 | Velcade ® + | 0.5 | iv | biwk |
| | | Custirsen (OGX) | 40 | ip | ip qd*5 + twk |

i.v.—intravenously, ip—intraperitoneally, twk—three times a week

Results

The treatment responses for Day 51 are summarized in Table 3 and presented in FIG. 1 and FIG. 2. FIG. 1 shows tumor growth curves as function of time. Treatment with custirsen at a dose of 40 mg/kg in both groups was stopped after 7 injections (5 consecutive+2 on the next week). On day 34, one mouse in custirsen 40 mg/kg group died and 2 mice in combination of custirsen 40 mg/kg with Velcade 0.5 mg/kg were euthanized due to more than 20% body weight loss. On Day 51, animals started to exit the study due to tumor size. All the treatments inhibited tumor growth compared to control group.

The effect of Velcade was moderate (28% TGI) and not statistically significant. Custersin alone inhibited tumor growth by 46% (p<0.05), but combined treatment of custirsen with Velcade had more significant dose-related activity. The study was terminated on day 71, when 1-3 mice left in each group. Survival curves are presented in FIG. 3.

TABLE 3
Summary of the results (day 51)

| | | | Δ mean | | | | | | |
|---|---|---|---|---|---|---|---|---|---|
| | | | tumor | | | | Max BW | | |
| | Dose, | | volume, | % TGI | No. | No. of | (mean) | No. of | No. og |
| Compound | mg/kg | Regimen | (mean ± se) | day 51 | of PR | CR | reduction | TRD | nTRD |
| 1. Vehicle | | i.p. qd*5, | 1228 ± 142 | | 0 | 0 | −1.3% | 0 | 0 |
| | | twk | | | | | day 34 | | |
| 2. Custirsen | 40 | ip qd*5, | 667 ± 87 | 46* | 0 | 0 | −4.7% | 1 | 0 |
| | | twk | | | | | day 34 | | |
| 3. Velcade® | 0.5 | iv biwk | 884 ± 356 | 28 | 0 | 0 | −0.7% | 0 | 0 |
| | | | | | | | day 34 | | |
| 4. Custirsen + | 20 | ip qd*5, | 658 ± 141 | 46** | 0 | 0 | −2.4% | 0 | 0 |
| Velcade® | | twk | | | | | day 34 | | |
| | 0.5 | iv biwk | | | | | | | |
| 5. Custirsen + | 40 | ip qd*5, | 336 ± 54 | 73***^{#} | 1 | 0 | −14.9% | 2 | 0 |
| Velcade® | | twk | | | | | day 34 | | |
| | 0.5 | iv biwk | | | | | | | |

*p < 0.05,
**p < 0.01,
***p < 0.001 (one-way Anova, Tukey post-hoc test)
^{#}p < 0.05 between groups 3 and 5
PR—partial response: tumor reduction below baseline measurement;
CR—complete response: elimination of the tumor;
TRD—treatment related death;
NTRD—non treatment related death.

Conclusions:
In RPMI 8226 xenograft model in female SCID mice, Velcade® at a dose of 0.5 mg/kg iv has moderate and not statistically significant effect;
Combined treatment with custirsen at both doses and Velcade® 0.5 mg/ml had better efficacy than Velcade® alone (p<0.05 for combination of custirsen 40 mg/kg with Velcade vs Velcade alone).

TABLE 4
Raw Data

| | | | | | | | | | | | | | | | Δ |
|---|---|---|---|---|---|---|---|---|---|---|---|---|---|---|---|
| | | Day 20 | | Day 27 | | Δ | | % of | Δ | Day 34, | | Δ | | % of | vol- |
| | | weight | volume | weight | volume | weight | Δ % | baseline | volume | weight | volume | weight | Δ % | baseline | ume |
| 1. Vehicle i.p. | 1 | 16.4 | 119 | 16.7 | 188 | 0.3 | 2.1 | 102.1 | 69.9 | 16.8 | 292 | 0.4 | 2.6 | 102.6 | 173.4 |
| qd*5, twk | 2 | 20.0 | 125 | 21.1 | 208 | 1.1 | 6.4 | 105.4 | 83.7 | 20.2 | 274 | 0.2 | 0.7 | 100.7 | 149.4 |
| | 3 | 21.4 | 133 | 22.1 | 205 | 0.7 | 3.1 | 103.1 | 72.6 | 21.1 | 400 | −0.3 | −1.5 | 98.5 | 267.4 |
| | 4 | 22.7 | 137 | 22.0 | 209 | −0.7 | −2.9 | 97.1 | 72.0 | 21.1 | 332 | −1.6 | −6.8 | 93.2 | 195.4 |
| | 5 | 16.9 | 139 | 16.7 | 258 | −0.2 | −1.2 | 98.8 | 118.7 | 16.6 | 478 | −0.3 | −1.6 | 98.4 | 339.3 |
| | 6 | 21.2 | 144 | 21.1 | 215 | −0.1 | −0.6 | 99.4 | 71.1 | 20.4 | 294 | −0.8 | −4.0 | 96.0 | 149.4 |
| | 7 | 21.6 | 152 | 21.9 | 203 | 0.3 | 1.4 | 101.4 | 51.6 | 21.5 | 275 | 0.0 | −0.1 | 99.9 | 122.8 |
| | 8 | 21.1 | 159 | 21.4 | 210 | 0.3 | 1.6 | 101.6 | 51.2 | 20.7 | 351 | −0.3 | −1.5 | 98.5 | 192.6 |
| | 9 | 19.1 | 162 | 19.5 | 218 | 0.5 | 2.5 | 102.5 | 56.8 | 19.0 | 359 | −0.1 | −0.4 | 99.6 | 197.7 |
| | 10 | 20.8 | 163 | 20.4 | 246 | −0.4 | −2.0 | 98.0 | 83.7 | 20.6 | 446 | −0.1 | −0.7 | 99.3 | 282.9 |
| | Mean | 20.1 | 143.1 | 20.3 | 216.2 | 0.2 | 0.9 | 100.9 | 73.1 | 19.8 | 350.1 | −0.3 | −1.3 | 98.7 | 207 |
| | n | 10 | 10 | 10 | 10 | 10 | 10 | 10 | 10 | 10 | 10 | 10 | 10 | 10 | 10 |
| | SD | 2.1 | 15.5 | 2.0 | 20.7 | 0.5 | 2.6 | 2.6 | 19.8 | 1.8 | 71.9 | 0.6 | 2.6 | 2.6 | 68.5 |
| | SE | 0.7 | 4.9 | 0.6 | 6.5 | 0.2 | 0.8 | 0.8 | 6.3 | 0.6 | 22.7 | 0.2 | 0.8 | 0.8 | 21.7 |
| 2. OGX 40 ip | 1 | 16.1 | 119 | 18.2 | 96 | 2.1 | 13.2 | 113.2 | −23.1 | 13.5 | 98 | −2.5 | −15.7 | 84.3 | −20.6 |
| qd*5, twk | 2 | 17.7 | 124 | 18.2 | 207 | 0.4 | 2.5 | 102.5 | 84.0 | 17.4 | 244 | −0.3 | −1.9 | 98.1 | 120.3 |
| | 3 | 17.1 | 125 | 18.0 | 171 | 0.8 | 4.8 | 104.8 | 46.2 | 17.1 | 223 | −0.1 | −0.4 | 99.6 | 97.9 |
| | 4 | 20.6 | 139 | 20.8 | 249 | 0.2 | 1.1 | 101.1 | 110.4 | 19.5 | 257 | −1.1 | −5.5 | 94.5 | 117.8 |
| | 5 | 19.7 | 141 | 19.7 | 222 | 0.1 | 0.3 | 100.3 | 81.2 | 19.1 | 305 | −0.5 | −2.7 | 97.3 | 163.9 |
| | 6 | 18.6 | 145 | 18.4 | 246 | −0.3 | −1.3 | 98.7 | 100.3 | 18.0 | 336 | −0.6 | −3.5 | 96.5 | 190.8 |
| | 7 | 18.4 | 151 | 18.9 | 319 | 0.4 | 2.4 | 102.4 | 168.8 | 18.2 | 371 | −0.2 | −1.1 | 98.9 | 219.9 |
| | 8 | 20.0 | 155 | 20.3 | 193 | 0.3 | 1.7 | 101.7 | 38.8 | 19.0 | 220 | −0.9 | −4.7 | 95.3 | 65.4 |
| | 9 | 18.4 | 162 | 18.6 | 217 | 0.2 | 0.8 | 100.8 | 55.3 | 17.2 | 266 | −1.2 | −6.7 | 93.3 | 104.3 |
| | 10 | 18.9 | 169 | 19.0 | 271 | 0.1 | 0.5 | 100.5 | 102.6 | 18.0 | 312 | −0.9 | −4.6 | 95.4 | 142.9 |
| | Mean | 18.5 | 142.8 | 19.0 | 219.2 | 0.4 | 2.6 | 102.6 | 76.4 | 17.7 | 263.1 | −0.8 | −4.7 | 95.3 | 120.3 |
| | n | 10 | 10 | 10 | 10 | 10 | 10 | 10 | 10 | 10 | 10 | 10 | 10 | 10 | 10 |
| | SD | 1.4 | 16.8 | 1.0 | 60.3 | 0.7 | 4.1 | 4.1 | 51.3 | 1.7 | 76.1 | 0.7 | 4.4 | 4.4 | 67.4 |
| | SE | 0.4 | 5.3 | 0.3 | 19.1 | 0.2 | 1.3 | 1.3 | 16.2 | 0.5 | 24.1 | 0.2 | 1.4 | 1.4 | 21.3 |
| 3. Velcade ® | 1 | 19.2 | 119 | 19.3 | 225 | 0.1 | 0.8 | 100.8 | 106.5 | 18.6 | 185 | −0.6 | −3.0 | 97.0 | 66.0 |
| 0.5 mg/kg | 2 | 20.1 | 123 | 20.3 | 260 | 0.2 | 1.0 | 101.0 | 137.0 | 20.2 | 232 | 0.1 | 0.3 | 100.3 | 109.0 |
| iv biwk | 3 | 19.2 | 133 | 19.7 | 235 | 0.5 | 2.6 | 102.6 | 102.2 | 19.1 | 255 | −0.1 | −0.6 | 99.4 | 121.9 |
| | 4 | 21.4 | 138 | 20.2 | 253 | −1.2 | −5.7 | 94.3 | 115.3 | 21.7 | 286 | 0.2 | 1.1 | 101.1 | 148.3 |
| | 5 | 20.2 | 141 | 21.9 | 206 | 1.7 | 8.6 | 108.6 | 65.3 | 19.9 | 266 | −0.3 | −1.3 | 98.7 | 124.7 |
| | 6 | 14.8 | 145 | 15.3 | 231 | 0.4 | 3.0 | 103.0 | 85.8 | 15.4 | 285 | 0.5 | 3.5 | 103.5 | 140.0 |
| | 7 | 18.2 | 149 | 18.6 | 149 | 0.5 | 2.7 | 102.7 | −0.6 | 18.1 | 272 | 0.0 | −0.1 | 99.9 | 123.0 |
| | 8 | 20.0 | 160 | 20.5 | 185 | 0.5 | 2.4 | 102.4 | 25.6 | 19.8 | 162 | −0.1 | −0.7 | 99.3 | 2.9 |
| | 9 | 19.7 | 162 | 19.6 | 225 | −0.1 | −0.7 | 99.3 | 63.7 | 20.6 | 343 | 0.9 | 4.5 | 104.5 | 181.4 |
| | 10 | 22.8 | 164 | 21.3 | 162 | −1.4 | −6.2 | −93.8 | −2.2 | 20.3 | 230 | −2.4 | −10.6 | 89.4 | 65.9 |
| | Mean | 19.5 | 143.2 | 19.7 | 213.1 | 0.1 | 0.8 | 100.8 | 69.9 | 19.4 | 251.6 | −0.2 | −0.7 | 99.3 | 108.3 |
| | n | 10 | 10 | 10 | 10 | 10 | 10 | 10 | 10 | 10 | 10 | 10 | 10 | 10 | 10 |
| | SD | 2.1 | 15.8 | 1.8 | 37.3 | 0.9 | 4.3 | 4.3 | 48.7 | 1.7 | 52.3 | 0.9 | 4.1 | 4.1 | 50.9 |
| | SE | 0.7 | 5.0 | 0.6 | 11.8 | 0.3 | 1.4 | 1.4 | 15.4 | 0.5 | 16.6 | 0.3 | 1.3 | 1.3 | 16.1 |
| 4. OGX 20 ip | 1 | 21.8 | 120 | 21.0 | 130 | −0.8 | −3.6 | 96.4 | 10.5 | 20.5 | 143 | −1.3 | −6.1 | 93.9 | 23.3 |
| qd*5, twk + | 2 | 18.9 | 124 | 19.6 | 201 | 0.7 | 3.8 | 103.8 | 77.2 | 18.6 | 272 | −0.3 | −1.5 | 98.5 | 147.9 |
| Velcade ® | 3 | 21.1 | 125 | 20.7 | 120 | −0.4 | −1.7 | 98.3 | −5.2 | 20.0 | 140 | −1.1 | −5.0 | 95.0 | 15.7 |
| 0.5 mg/kg | 4 | 19.7 | 134 | 20.2 | 150 | 0.6 | 2.8 | 102.8 | 16.6 | 19.3 | 141 | −0.4 | −2.0 | 98.0 | 6.9 |
| | 5 | 19.5 | 143 | 20.9 | 231 | 1.5 | 7.6 | 107.6 | 87.6 | 20.0 | 259 | 0.5 | 2.6 | 102.6 | 115.7 |
| | 6 | 19.0 | 146 | 21.4 | 250 | 2.3 | 12.2 | 112.2 | 103.9 | 20.8 | 406 | 1.7 | 9.1 | 109.1 | 259.6 |
| | 7 | 19.2 | 151 | 20.3 | 301 | 1.1 | 5.9 | 105.9 | 150.3 | 19.6 | 284 | 0.4 | 2.0 | 102.0 | 133.4 |
| | 8 | 22.1 | 155 | 22.0 | 249 | −0.1 | −0.3 | 99.7 | 94.2 | 19.6 | 263 | −2.4 | −11.0 | 89.0 | 108.3 |
| | 9 | 20.3 | 161 | 20.5 | 207 | 0.2 | 1.1 | 101.1 | 46.6 | 18.9 | 290 | −1.4 | −6.9 | 93.1 | 128.7 |
| | 10 | 21.1 | 171 | 20.8 | 246 | −0.2 | −1.1 | 98.9 | 74.9 | 20.0 | 257 | −1.1 | −5.0 | 95.0 | 86.0 |
| | Mean | 20.2 | 142.8 | 20.7 | 208.5 | 0.5 | 2.7 | 102.7 | 65.7 | 19.7 | 245.4 | −0.5 | −2.4 | 97.6 | 102.6 |
| | n | 10 | 10 | 10 | 10 | 10 | 10 | 10 | 10 | 10 | 10 | 10 | 10 | 10 | 10 |
| | SD | 1.2 | 17.1 | 0.7 | 59.0 | 0.9 | 4.8 | 4.8 | 48.3 | 0.7 | 83.7 | 1.2 | 5.8 | 5.8 | 75.9 |
| | SE | 0.4 | 5.4 | 0.2 | 18.6 | 0.3 | 1.5 | 1.5 | 15.3 | 0.2 | 26.5 | 0.4 | 1.8 | 1.8 | 24.0 |
| 5.OGX 40 ip | 1 | 20.1 | 123 | 20.3 | 144 | 0.2 | 1.1 | 101.1 | 21.1 | 17.1 | 178 | −2.9 | −14.6 | 85.4 | 55.8 |
| qd*5, twk + | 2 | 21.4 | 124 | 21.2 | 173 | −0.2 | −0.8 | 99.2 | 49.3 | 19.2 | 153 | −2.2 | −10.3 | 89.7 | 29.3 |
| Velcade ® | 3 | 21.5 | 125 | 22.1 | 168 | 0.6 | 3.0 | 103.0 | 42.8 | 17.7 | 197 | −3.8 | −17.8 | 82.2 | 72.5 |
| 0.5 mg/kg | 4 | 20.0 | 134 | 20.2 | 71 | 0.2 | 0.7 | 100.7 | −63.5 | 16.7 | 95 | −3.3 | −16.7 | 83.3 | −39.5 |
| | 5 | 21.3 | 139 | 19.7 | 197 | −1.7 | −7.8 | 92.2 | 57.8 | 15.6 | 191 | −5.7 | −26.6 | 73.4 | 51.6 |
| | 6 | 19.9 | 147 | 19.2 | 188 | −0.8 | −3.8 | 96.2 | 41.4 | 14.8 | 152 | −5.2 | −26.0 | 74.0 | 5.0 |
| | 7 | 19.7 | 152 | 20.3 | 154 | 0.6 | 2.8 | 102.8 | 2.4 | 18.9 | 192 | −0.8 | −4.0 | 96.0 | 40.5 |
| | 8 | 23.1 | 157 | 22.5 | 216 | −0.6 | −2.6 | 97.4 | 58.8 | 20.6 | 200 | −2.5 | −10.6 | 89.4 | 42.9 |
| | 9 | 19.7 | 161 | 19.8 | 203 | 0.1 | 0.4 | 100.4 | 41.9 | 16.7 | 181 | −3.1 | −15.5 | 84.5 | 19.5 |
| | 10 | 18.6 | 162 | 19.3 | 266 | 0.7 | 3.8 | 103.8 | 103.5 | 17.3 | 214 | −1.3 | −6.8 | 93.2 | −51.6 |
| | Mean | 20.5 | 142.3 | 20.4 | 177.9 | −0.1 | −0.3 | 99.7 | 35.6 | 17.5 | 175.2 | −3.1 | −14.9 | 85.1 | 32.9 |
| | n | 10 | 10 | 10 | 10 | 10 | 10 | 10 | 10 | 10 | 10 | 10 | 10 | 10 | 10 |
| | SD | 1.3 | 15.6 | 1.1 | 51.2 | 0.7 | 3.6 | 3.6 | 43.6 | 1.7 | 34.5 | 1.5 | 7.4 | 7.4 | 31.9 |
| | SE | 0.4 | 4.9 | 0.4 | 16.2 | 0.2 | 1.1 | 1.1 | 13.8 | 0.6 | 10.9 | 0.5 | 2.3 | 2.3 | 10.1 |

| | | Day 41 | | | | % of | | Day 45 | | | | % of | |
|---|---|---|---|---|---|---|---|---|---|---|---|---|---|
| | | weight | volume | Δ weight | Δ % | baseline | Δ volume | weight | volume | Δ weight | Δ % | baseline | Δ volume |
| 1. Vehicle i.p. qd*5, | 1 | 17.2 | 370 | 0.8 | 4.6 | 104.6 | 251.4 | 17.7 | 371 | 1.4 | 8.2 | 108.2 | 252.2 |
| twk | 2 | 20.8 | 564 | 0.8 | 3.8 | 103.8 | 429.6 | 21.2 | 835 | 1.1 | 5.7 | 105.7 | 710.8 |
| | 3 | 21.6 | 688 | 0.2 | 1.1 | 101.1 | 555.7 | 22.2 | 945 | 0.8 | 3.7 | 103.7 | 812.5 |
| | 4 | 22.5 | 659 | −0.2 | −0.7 | 99.3 | 522.1 | 22.6 | 880 | 0.0 | −0.2 | 99.8 | 742.9 |
| | 5 | 17.3 | 816 | 0.4 | 2.6 | 102.6 | 677.2 | 17.8 | 1118 | 0.9 | 5.6 | 105.6 | 979.0 |
| | 6 | 21.0 | 577 | −0.2 | −0.9 | 99.1 | 432.6 | 21.7 | 811 | 0.4 | 2.1 | 102.1 | 666.8 |
| | 7 | 22.2 | 402 | 0.6 | 2.9 | 102.9 | 250.2 | 22.9 | 475 | 1.3 | 6.1 | 106.1 | 323.2 |
| | 8 | 21.3 | 505 | 0.2 | 1.0 | 101.0 | 346.5 | 21.8 | 795 | 0.7 | 3.5 | 103.5 | 636.2 |
| | 9 | 19.4 | 735 | 0.4 | 2.0 | 102.0 | 573.1 | 19.9 | 844 | 0.8 | 4.3 | 104.3 | 682.6 |
| | 10 | 21.2 | 977 | 0.4 | 2.0 | 102.0 | 814.1 | 22.7 | 1341 | 1.9 | 9.3 | 109.3 | 1178.6 |
| | Mean | 20.4 | 628.3 | 0.3 | 1.8 | 101.8 | 485 | 21.0 | 841.6 | 0.9 | 4.8 | 104.8 | 698 |
| | n | 10 | 10 | 10 | 10 | 10 | 10 | 10 | 10 | 10 | 10 | 10 | 10 |
| | SD | 1.9 | 186.8 | 0.3 | 1.8 | 1.8 | 180.8 | 1.9 | 278.4 | 0.5 | 2.8 | 2.6 | 272.7 |
| | SE | 0.6 | 59.1 | 0.1 | 0.6 | 0.6 | 57.2 | 0.6 | 88.0 | 0.2 | 0.9 | 0.9 | 86.2 |
| 2. OGX 40 ip qd*5, | 1 | | | | | | | | | | | | |
| twk | 2 | 18.8 | 410 | 1.1 | 6.0 | 106.0 | 286.2 | 19.2 | 511 | 1.4 | 8.1 | 108.1 | 388.0 |
| | 3 | 17.9 | 295 | 0.8 | 4.4 | 104.4 | 170.4 | 18.8 | 272 | 1.7 | 9.8 | 109.8 | 147.2 |
| | 4 | 20.9 | 322 | 0.3 | 1.6 | 101.6 | 183.5 | 21.9 | 579 | 1.3 | 6.1 | 106.1 | 440.5 |
| | 5 | 19.8 | 454 | 0.1 | 0.4 | 100.4 | 313.0 | 20.5 | 587 | 0.8 | 4.1 | 104.1 | 446.1 |
| | 6 | 18.7 | 474 | 0.1 | 0.3 | 100.3 | 328.4 | 19.1 | 584 | 0.5 | 2.7 | 102.7 | 439.0 |
| | 7 | 19.5 | 560 | 1.1 | 5.9 | 105.9 | 409.6 | 20.6 | 826 | 2.1 | 11.4 | 111.4 | 675.2 |
| | 8 | 19.9 | 267 | −0.1 | −0.5 | 99.5 | 112.3 | 21.1 | 467 | 1.1 | 5.6 | 105.6 | 313.0 |
| | 9 | 17.6 | 320 | −0.8 | −4.2 | 95.8 | 157.8 | 18.7 | 428 | 0.3 | 1.5 | 101.5 | 266.0 |
| | 10 | 18.9 | 457 | 0.0 | 0.1 | 100.1 | 288.2 | 19.1 | 490 | 0.2 | 1.3 | 101.3 | 321.7 |
| | Mean | 19.1 | 395.4 | 0.3 | 1.5 | 101.5 | 249.9 | 19.9 | 527.3 | 1.0 | 5.6 | 105.6 | 381.9 |
| | n | 9 | 9 | 9 | 9 | 9 | 9 | 9 | 9 | 9 | 9 | 9 | 9 |
| | SD | 1.0 | 99.0 | 0.6 | 3.3 | 3.3 | 97.8 | 1.1 | 149.6 | 0.6 | 3.6 | 3.6 | 147.4 |
| | SE | 0.3 | 33.0 | 0.2 | 1.1 | 1.1 | 32.6 | 0.4 | 49.9 | 0.2 | 1.2 | 1.2 | 49.1 |
| 3. Velcade ® 0.5 | 1 | 20.3 | 289 | 1.1 | 5.6 | 105.6 | 170.3 | 20.6 | 430 | 1.5 | 7.6 | 107.6 | 311.5 |
| mg/kg iv biwk | 2 | 20.8 | 481 | 0.7 | 3.5 | 103.5 | 358.8 | 21.0 | 667 | 0.8 | 4.2 | 104.2 | 544.2 |
| | 3 | 20.1 | 501 | 0.9 | 4.6 | 104.6 | 368.2 | 20.5 | 621 | 1.3 | 7.0 | 107.0 | 488.4 |
| | 4 | 21.9 | 387 | 0.5 | 2.3 | 102.3 | 249.1 | 22.8 | 507 | 1.4 | 6.5 | 106.5 | 368.7 |
| | 5 | 20.8 | 491 | 0.6 | 3.0 | 103.0 | 349.9 | 21.1 | 733 | 1.0 | 4.8 | 104.8 | 592.6 |
| | 6 | 14.6 | 443 | −0.2 | −1.5 | 98.5 | 297.3 | 15.0 | 495 | 0.2 | 1.1 | 101.1 | 350.1 |
| | 7 | 18.5 | 475 | 0.4 | 2.0 | 102.0 | 325.8 | 19.3 | 663 | 1.2 | 6.4 | 106.4 | 514.1 |
| | 8 | 20.7 | 269 | 0.8 | 3.8 | 103.8 | 109.0 | 21.3 | 351 | 1.4 | 6.8 | 106.8 | 191.8 |
| | 9 | 20.5 | 528 | 0.8 | 4.2 | 104.2 | 366.6 | 21.2 | 744 | 1.5 | 7.5 | 107.5 | 582.0 |
| | 10 | 21.2 | 384 | −1.5 | −6.7 | 93.3 | 200.2 | 22.1 | 539 | −0.6 | −2.9 | 97.1 | 374.8 |
| | Mean | 19.9 | 422.8 | 0.4 | 2.1 | 102.1 | 279.5 | 20.5 | 575.1 | 1.0 | 4.9 | 104.9 | 431.8 |
| | n | 10 | 10 | 10 | 10 | 10 | 10 | 10 | 10 | 10 | 10 | 10 | 10 |
| | SD | 2.1 | 91.2 | 0.8 | 3.6 | 3.6 | 92.7 | 2.1 | 131.3 | 0.7 | 3.4 | 3.4 | 132.1 |
| | SE | 0.7 | 28.8 | 0.2 | 1.1 | 1.1 | 29.3 | 0.7 | 41.5 | 0.2 | 1.1 | 1.1 | 41.8 |
| 4. OGX 20 ip qd*5, | 1 | 20.8 | 182 | −1.0 | −4.8 | 95.2 | 62.4 | 21.8 | 195 | 0.0 | 0.0 | 100.0 | 75.6 |
| twk + Velcade ® | 2 | 19.2 | 664 | 0.3 | 1.7 | 101.7 | 540.1 | 20.1 | 616 | 1.2 | 6.3 | 106.3 | 492.8 |
| 0.5 mg/kg iv biwk | 3 | 20.8 | 240 | −0.2 | −1.0 | 99.0 | 115.4 | 21.4 | 261 | 0.3 | 1.6 | 101.6 | 136.5 |
| | 4 | 20.2 | 267 | 0.5 | 2.5 | 102.5 | 133.1 | 21.0 | 416 | 1.3 | 6.7 | 106.7 | 282.2 |
| | 5 | 21.1 | 458 | 1.6 | 8.3 | 108.3 | 315.5 | 21.2 | 602 | 1.7 | 8.8 | 108.8 | 459.4 |
| | 6 | 21.0 | 438 | 1.9 | 10.1 | 110.1 | 291.9 | 21.1 | 685 | 2.0 | 10.7 | 110.7 | 538.6 |
| | 7 | 20.0 | 314 | 0.8 | 4.4 | 104.4 | 163.0 | 20.7 | 364 | 1.5 | 7.9 | 107.9 | 213.7 |
| | 8 | 17.7 | 269 | −4.4 | −19.8 | 80.2 | 113.7 | 20.1 | 211 | −2.0 | −8.9 | 91.1 | 56.5 |
| | 9 | 20.1 | 629 | −0.1 | −0.7 | 99.3 | 468.2 | 20.8 | 941 | 0.5 | 2.6 | 102.6 | 780.1 |
| | 10 | 20.8 | 385 | −0.2 | −1.0 | 99.0 | 214.1 | 22.0 | 426 | 1.0 | 4.6 | 104.6 | 255.3 |
| | Mean | 20.2 | 384.6 | −0.1 | 0.0 | 100.0 | 241.7 | 21.0 | 471.9 | 0.8 | 4.0 | 104.0 | 329.1 |
| | n | 10 | 10 | 10 | 10 | 10 | 10 | 10 | 10 | 10 | 10 | 10 | 10 |
| | SD | 1.0 | 163.4 | 1.8 | 8.3 | 8.3 | 160.4 | 0.6 | 237.8 | 1.1 | 5.6 | 5.6 | 232.8 |
| | SE | 0.3 | 51.7 | 0.6 | 2.6 | 2.6 | 50.7 | 0.2 | 75.2 | 0.4 | 1.8 | 1.8 | 73.6 |
| 5. OGX 40 ip qd*5, | 1 | 19.7 | 169 | −0.4 | −2.0 | 98.0 | 46.1 | 20.4 | 166 | 0.4 | 1.8 | 101.8 | 43.2 |
| twk + Velcade ® | 2 | 20.3 | 203 | −1.1 | −4.9 | 95.1 | 79.4 | 20.9 | 224 | −0.5 | −2.4 | 97.6 | 100.5 |
| 0.5 mg/kg iv biwk | 3 | 20.7 | 195 | −0.8 | −3.7 | 96.3 | 70.3 | 21.9 | 318 | 0.4 | 1.7 | 101.7 | 193.3 |
| | 4 | 19.5 | 113 | −0.5 | −2.5 | 97.5 | −20.7 | 20.1 | 82 | 0.1 | 0.3 | 100.3 | −51.8 |
| | 5 | | | | | | | | | | | | |
| | 6 | | | | | | | | | | | | |
| | 7 | 20.5 | 223 | 0.8 | 4.2 | 104.2 | 71.7 | 21.1 | 319 | 1.3 | 6.7 | 106.7 | 167.9 |
| | 8 | 21.8 | 202 | −1.3 | −5.5 | 94.5 | 44.9 | 22.3 | 311 | −0.8 | −3.3 | 96.7 | 153.3 |
| | 9 | 19.9 | 244 | 0.2 | 0.8 | 100.8 | 83.2 | 21.1 | 302 | 1.4 | 7.3 | 107.3 | 141.0 |
| | 10 | 19.3 | 230 | 0.7 | 3.8 | 103.8 | 67.6 | 20.0 | 308 | 1.4 | 7.6 | 107.6 | 146.2 |
| | Mean | 20.2 | 197.5 | −0.3 | −1.2 | 98.8 | 55.3 | 21.0 | 253.9 | 0.5 | 2.5 | 102.5 | 111.7 |
| | n | 8 | 8 | 8 | 8 | 8 | 8 | 8 | 8 | 8 | 8 | 8 | 8 |
| | SD | 0.8 | 41.1 | 0.8 | 3.8 | 3.8 | 33.8 | 0.8 | 88.7 | 0.9 | 4.3 | 4.3 | 80.3 |
| | SE | 0.3 | 14.5 | 0.3 | 1.3 | 1.3 | 11.9 | 0.3 | 31.4 | 0.3 | 1.5 | 1.5 | 28.4 |

| | | Day 48 | | Δ | | % of | | Day 51 | | | | % of | Δ |
|---|---|---|---|---|---|---|---|---|---|---|---|---|---|
| | | weight | volume | weight | Δ % | baseline | Δ volume | weight | volume | Δ weight | Δ % | baseline | volume |
| 1. Vehicle i.p. qd*5, twk | 1 | 16.6 | 510 | 0.2 | 1.2 | 101.2 | 391.5 | 16.6 | 538 | 0.2 | 1.5 | 101.5 | 419.2 |
| | 2 | 20.8 | 1000 | 0.7 | 3.5 | 103.5 | 875.0 | 21.5 | 1330 | 1.4 | 7.1 | 107.1 | 1205.0 |
| | 3 | 22.0 | 1111 | 0.6 | 3.0 | 103.0 | 978.8 | 22.0 | 1487 | 0.6 | 2.9 | 102.9 | 1354.7 |
| | 4 | 22.5 | 1140 | −0.1 | −0.5 | 99.5 | 1003.3 | 22.8 | 1406 | 0.2 | 0.8 | 100.8 | 1270.6 |
| | 5 | 17.4 | 1409 | 0.6 | 3.4 | 103.4 | 1269.6 | 18.3 | 2054 | 1.4 | 8.2 | 108.2 | 1915.0 |
| | 6 | 21.1 | 1002 | −0.1 | −0.7 | 99.3 | 857.5 | 21.4 | 1241 | 0.2 | 0.8 | 100.8 | 1096.6 |
| | 7 | 22.5 | 720 | 1.0 | 4.5 | 104.5 | 567.9 | 22.6 | 794 | 1.0 | 4.8 | 104.8 | 642.1 |
| | 8 | 21.5 | 986 | 0.4 | 2.0 | 102.0 | 827.1 | 21.7 | 1447 | 0.7 | 3.1 | 103.1 | 1289.0 |
| | 9 | 20.1 | 1125 | 1.0 | 5.4 | 105.4 | 963.5 | 20.1 | 1482 | 1.0 | 5.5 | 105.5 | 1320.2 |
| | 10 | 22.5 | 1388 | 1.7 | 8.4 | 108.4 | 1225.2 | 23.3 | 1934 | 2.6 | 12.4 | 112.4 | 1771.2 |
| | Mean | 20.7 | 1039.0 | 0.6 | 3.0 | 103.0 | 896 | 21.0 | 1371.4 | 0.9 | 4.7 | 104.7 | 1228 |
| | n | 10 | 10 | 10 | 10 | 10 | 10 | 10 | 10 | 10 | 10 | 10 | 10 |
| | SD | 2.1 | 272.6 | 0.6 | 2.7 | 2.7 | 266.6 | 2.1 | 454.9 | 0.7 | 3.7 | 3.7 | 448.8 |
| | SE | 0.7 | 86.2 | 0.2 | 0.9 | 0.9 | 84.3 | 0.7 | 143.9 | 0.2 | 1.2 | 1.2 | 141.9 |
| 2. OGX 40 ip qd*5, twk | 1 | | | | | | | | | | | | |
| | 2 | 18.73 | 495 | 1.0 | 5.6 | 105.6 | 371.9 | 18.40 | 687 | 0.7 | 3.8 | 103.8 | 563.6 |
| | 3 | 18.93 | 354 | 1.8 | 10.6 | 110.6 | 229.1 | 18.79 | 392 | 1.7 | 9.8 | 109.8 | 267.5 |
| | 4 | 21.36 | 689 | 0.8 | 3.7 | 103.7 | 550.6 | 21.71 | 817 | 1.1 | 5.4 | 105.4 | 678.0 |
| | 5 | 20.43 | 763 | 0.8 | 3.8 | 103.8 | 621.8 | 20.17 | 923 | 0.5 | 2.5 | 102.5 | 781.6 |
| | 6 | 19.03 | 717 | 0.4 | 2.1 | 102.1 | 571.9 | 19.13 | 1031 | 0.5 | 2.7 | 102.7 | 885.6 |
| | 7 | 20.83 | 1076 | 2.4 | 13.0 | 113.0 | 925.7 | 21.06 | 1324 | 2.6 | 14.2 | 114.2 | 1173.4 |
| | 8 | 20.79 | 483 | 0.8 | 4.2 | 104.2 | 329.0 | 20.78 | 603 | 0.8 | 4.2 | 104.2 | 448.7 |
| | 9 | 18.98 | 593 | 0.6 | 3.2 | 103.2 | 431.2 | 18.47 | 734 | 0.1 | 0.4 | 100.4 | 571.8 |
| | 10 | 19.13 | 671 | 0.2 | 1.2 | 101.2 | 502.7 | 19.87 | 804 | 1.0 | 5.1 | 105.1 | 635.5 |
| | Mean | 19.8 | 649.2 | 1.0 | 5.3 | 105.3 | 503.8 | 19.8 | 812.8 | 1.0 | 5.3 | 105.3 | 667.3 |
| | n | 9 | 9 | 9 | 9 | 9 | 9 | 9 | 9 | 9 | 9 | 9 | 9 |
| | SD | 1.0 | 207.3 | 0.7 | 3.9 | 3.9 | 202.3 | 1.2 | 265.5 | 0.8 | 4.2 | 4.2 | 261.1 |
| | SE | 0.3 | 69.1 | 0.2 | 1.3 | 1.3 | 67.4 | 0.4 | 88.5 | 0.3 | 1.4 | 1.4 | 87.0 |
| 3. Velcade ® 0.5 mg/kg | 1 | 20.0 | 520 | 0.8 | 4.2 | 104.2 | 401.6 | 20.2 | 767 | 1.0 | 5.0 | 105.0 | 648.2 |
| iv biwk | 2 | 20.7 | 868 | 0.6 | 3.1 | 103.1 | 745.2 | 20.8 | 1043 | 0.7 | 3.5 | 103.5 | 920.6 |
| | 3 | 19.7 | 757 | 0.5 | 2.8 | 102.8 | 624.2 | 20.5 | 1313 | 1.3 | 6.9 | 106.9 | 1179.9 |
| | 4 | 21.6 | 647 | 0.1 | 0.7 | 100.7 | 508.9 | 22.3 | 774 | 0.8 | 3.9 | 103.9 | 636.4 |
| | 5 | 20.8 | 1060 | 0.7 | 3.3 | 103.3 | 919.1 | 21.5 | 1432 | 1.4 | 6.8 | 106.8 | 1291.7 |
| | 6 | 14.8 | 549 | −0.1 | −0.3 | 99.7 | 403.8 | 15.3 | 619 | 0.4 | 2.8 | 102.8 | 474.1 |
| | 7 | 18.6 | 687 | 0.4 | 2.4 | 102.4 | 537.5 | 19.2 | 917 | 1.0 | 5.7 | 105.7 | 768.2 |
| | 8 | 20.7 | 470 | 0.8 | 3.8 | 103.8 | 310.7 | 21.2 | 578 | 1.2 | 5.9 | 105.9 | 418.7 |
| | 9 | 20.0 | 1151 | 0.3 | 1.8 | 101.8 | 988.8 | 20.6 | 1635 | 0.9 | 4.6 | 104.6 | 1473.4 |
| | 10 | 21.1 | 789 | −1.6 | −7.1 | 92.9 | 625.7 | 21.3 | 1195 | −1.5 | −6.6 | 93.4 | 1031.3 |
| | Mean | 19.8 | 749.8 | 0.3 | 1.5 | 101.5 | 606.6 | 20.3 | 1027.5 | 0.7 | 3.8 | 103.8 | 884.2 |
| | n | 10 | 10 | 10 | 10 | 10 | 10 | 10 | 10 | 10 | 10 | 10 | 10 |
| | SD | 2.0 | 225.6 | 0.7 | 3.3 | 3.3 | 223.3 | 1.9 | 358.3 | 0.8 | 3.9 | 3.9 | 355.7 |
| | SE | 0.6 | 71.3 | 0.2 | 1.0 | 1.0 | 70.6 | 0.6 | 113.3 | 0.3 | 1.2 | 1.2 | 112.5 |
| 4. OGX 20 ip qd*5, twk + | 1 | 21.0 | 306 | −0.8 | −3.7 | 96.3 | 186.2 | 21.2 | 272 | −0.6 | −2.8 | 97.2 | 152.1 |
| Velcade ® 0.5 mg/kg | 2 | 19.3 | 802 | 0.4 | 2.3 | 102.3 | 678.0 | 19.7 | 1015 | 0.8 | 4.3 | 104.3 | 891.2 |
| iv biwk | 3 | 20.8 | 349 | −0.3 | −1.3 | 98.7 | 223.7 | 21.0 | 554 | −0.1 | −0.4 | 99.6 | 429.5 |
| | 4 | 19.9 | 471 | 0.2 | 1.2 | 101.2 | 337.6 | 19.8 | 591 | 0.1 | 0.8 | 100.8 | 457.4 |
| | 5 | 20.8 | 1061 | 1.3 | 6.7 | 106.7 | 917.9 | 20.7 | 1360 | 1.2 | 6.1 | 106.1 | 1216.6 |
| | 6 | 20.8 | 866 | 1.8 | 9.5 | 109.5 | 719.3 | 21.1 | 1076 | 2.1 | 11.0 | 111.0 | 929.7 |
| | 7 | 19.7 | 379 | 0.5 | 2.6 | 102.8 | 228.4 | 19.5 | 494 | 0.3 | 1.6 | 101.6 | 343.6 |
| | 8 | 19.5 | 288 | −2.6 | −11.6 | 88.4 | 133.2 | 20.2 | 380 | −1.9 | −8.5 | 91.5 | 224.9 |
| | 9 | 20.3 | 1316 | 0.0 | 0.0 | 100.0 | 1155.3 | 19.9 | 1641 | −0.3 | −1.7 | 98.3 | 1480.1 |
| | 10 | 20.8 | 476 | −0.3 | −1.4 | 98.6 | 305.0 | 21.0 | 623 | −0.1 | −0.4 | 99.8 | 452.3 |
| | Mean | 20.3 | 631.3 | 0.0 | 0.4 | 100.4 | 488.5 | 20.4 | 800.6 | 0.2 | 1.0 | 101.0 | 657.7 |
| | n | 10 | 10 | 10 | 10 | 10 | 10 | 10 | 10 | 10 | 10 | 10 | 10 |
| | SD | 0.6 | 358.2 | 1.2 | 5.8 | 5.8 | 354.3 | 0.7 | 450.6 | 1.1 | 5.3 | 5.3 | 446.4 |
| | SE | 0.2 | 113.3 | 0.4 | 1.8 | 1.8 | 112.1 | 0.2 | 142.5 | 0.3 | 1.7 | 1.7 | 141.2 |
| 5. OGX 40 ip qd*5, twk + | 1 | 19.9 | 202 | −0.2 | −0.9 | 99.1 | 79.3 | 20.3 | 292 | 0.3 | 1.3 | 101.3 | 168.9 |
| Velcade ® 0.5 mg/kg | 2 | 20.5 | 331 | −0.9 | −4.0 | 96.0 | 207.3 | 21.5 | 495 | 0.1 | 0.5 | 100.5 | 371.6 |
| iv biwk | 3 | 21.9 | 370 | 0.4 | 1.7 | 101.7 | 245.3 | 22.3 | 498 | 0.8 | 3.6 | 103.6 | 372.8 |
| | 4 | | | | | | | | | | | | |
| | 5 | | | | | | | | | | | | |
| | 6 | 19.6 | 153 | −0.5 | −2.3 | 97.7 | 19.2 | 20.4 | 219 | 0.3 | 1.6 | 101.6 | 85.3 |
| | 7 | 20.9 | 387 | 1.2 | 6.1 | 106.1 | 235.7 | 21.6 | 718 | 1.9 | 9.4 | 109.4 | 566.1 |
| | 8 | 21.9 | 319 | −1.2 | −5.2 | 94.8 | 161.4 | 22.0 | 454 | −1.1 | −4.6 | 95.4 | 296.5 |
| | 9 | 20.1 | 423 | 0.4 | 2.2 | 102.2 | 262.0 | 20.3 | 615 | 0.6 | 2.9 | 102.9 | 453.5 |
| | 10 | 19.5 | 417 | 0.9 | 4.9 | 104.9 | 254.5 | 20.2 | 539 | 1.6 | 8.4 | 108.4 | 377.2 |
| | Mean | 20.5 | 325.3 | 0.0 | 0.3 | 100.3 | 183.1 | 21.1 | 478.7 | 0.6 | 2.9 | 102.9 | 336.5 |
| | n | 8 | 8 | 8 | 8 | 8 | 8 | 8 | 8 | 8 | 8 | 8 | 8 |
| | SD | 0.9 | 99.0 | 0.8 | 4.1 | 4.1 | 90.0 | 0.9 | 161.6 | 0.9 | 4.5 | 4.5 | 152.8 |
| | SE | 0.3 | 35.0 | 0.3 | 1.4 | 1.4 | 31.8 | 0.3 | 57.1 | 0.3 | 1.6 | 1.6 | 54.0 |
| | | Day 58 | | Δ | | % of | | Day 63 | | | | % of | Δ |
| | | weight | volume | weight | Δ % | baseline | Δ volume | weight | volume | Δ weight | Δ % | baseline | volume |
| 1. Vehicle i.p. qd*5, twk | 1 | 17.5 | 830 | 1.1 | 6.8 | 106.8 | 711.8 | 17.3 | 1255 | 0.9 | 5.8 | 105.8 | 1136.3 |
| | 2 | 22.4 | 1934 | 2.4 | 11.8 | 111.8 | 1809.7 | | | | | | |
| | 3 | 23.2 | 2416 | 1.8 | 8.2 | 108.2 | 2283.1 | | | | | | |
| | 4 | 23.8 | 3067 | 1.2 | 5.3 | 105.3 | 2929.9 | | | | | | |
| | 5 | | | | | | | | | | | | |
| | 6 | 22.4 | 1614 | 1.2 | 5.7 | 105.7 | 1469.9 | | | | | | |
| | 7 | 23.2 | 856 | 1.7 | 7.7 | 107.7 | 704.4 | 22.7 | 1007 | 1.1 | 5.3 | 105.3 | 855.2 |
| | 8 | 22.9 | 2006 | 1.8 | 8.6 | 108.6 | 1847.0 | | | | | | |
| | 9 | 20.8 | 2331 | 1.8 | 9.2 | 109.2 | 2169.0 | | | | | | |
| | 10 | | | | | | | | | | | | |
| | Mean | 22.0 | 1881.7 | 1.6 | 7.9 | 107.9 | 1741 | 20.2 | 1130.9 | 1.0 | 5.5 | 105.5 | 996 |
| | n | 8 | 8 | 8 | 8 | 8 | 8 | 2 | 2 | 2 | 2 | 2 | 2 |
| | SD | 2.0 | 768.9 | 0.4 | 2.1 | 2.1 | 766.6 | 3.8 | 175.2 | 0.1 | 0.4 | 0.4 | 198.8 |
| | SE | 0.7 | 271.8 | 0.1 | 0.7 | 0.7 | 271.0 | 2.7 | 123.9 | 0.1 | 0.3 | 0.3 | 140.5 |
| 2. OGX 40 ip qd*5, twk | 1 | | | | | | | | | | | | |
| | 2 | 18.72 | 797 | 1.0 | 5.6 | 105.6 | 673.0 | 18.57 | 1127 | 0.8 | 4.7 | 104.7 | 1003.4 |
| | 3 | 18.84 | 560 | 1.7 | 10.0 | 110.0 | 435.6 | 19.25 | 848 | 2.1 | 12.4 | 112.4 | 723.2 |
| | 4 | 22.31 | 1319 | 1.7 | 8.4 | 108.4 | 1180.5 | 22.39 | 2000 | 1.8 | 8.7 | 108.7 | 1861.0 |
| | 5 | 20.77 | 1418 | 1.1 | 5.5 | 105.5 | 1277.2 | 21.04 | 2185 | 1.4 | 6.9 | 106.9 | 2043.7 |
| | 6 | 19.53 | 1693 | 0.9 | 4.8 | 104.8 | 1547.4 | | | | | | |
| | 7 | 21.42 | 1970 | 3.0 | 16.2 | 116.2 | 1819.3 | | | | | | |
| | 8 | 21.45 | 1060 | 1.5 | 7.5 | 107.5 | 905.2 | 22.19 | 1918 | 2.2 | 11.2 | 111.2 | 1763.0 |
| | 9 | 19.51 | 1218 | 1.1 | 6.0 | 106.0 | 1056.5 | 19.81 | 1519 | 1.4 | 7.7 | 107.7 | 1357.1 |
| | 10 | 20.43 | 1475 | 1.5 | 8.1 | 108.1 | 1306.4 | 20.11 | 2426 | 1.2 | 6.4 | 106.4 | 2257.1 |
| | Mean | 20.3 | 1278.9 | 1.5 | 8.0 | 108.0 | 1133.5 | 20.5 | 1717.4 | 1.6 | 8.3 | 108.3 | 1572.6 |
| | n | 9 | 9 | 9 | 9 | 9 | 9 | 7 | 7 | 7 | 7 | 7 | 7 |
| | SD | 1.3 | 434.3 | 0.6 | 3.5 | 3.5 | 426.0 | 1.5 | 575.1 | 0.5 | 2.7 | 2.7 | 563.0 |
| | SE | 0.4 | 144.8 | 0.2 | 1.2 | 1.2 | 142.0 | 0.5 | 217.4 | 0.2 | 1.0 | 1.0 | 212.8 |
| 3. Velcade ® 0.5 mg/kg | 1 | 22.2 | 1019 | 3.0 | 15.7 | 115.7 | 900.3 | 21.8 | 1484 | 2.6 | 13.6 | 113.6 | 1365.3 |
| iv biwk | 2 | 21.9 | 1659 | 1.7 | 8.7 | 108.7 | 1536.1 | | | | | | |
| | 3 | 21.2 | 1885 | 2.0 | 10.6 | 110.6 | 1752.5 | | | | | | |
| | 4 | 22.7 | 779 | 1.3 | 5.8 | 105.8 | 640.9 | 23.0 | 881 | 1.6 | 7.4 | 107.4 | 742.6 |
| | 5 | 22.6 | 2196 | 2.5 | 12.3 | 112.3 | 2055.5 | | | | | | |
| | 6 | 15.5 | 721 | 0.7 | 4.7 | 104.7 | 575.7 | 16.3 | 1166 | 1.5 | 9.9 | 109.9 | 1020.7 |
| | 7 | 20.2 | 1500 | 2.1 | 11.4 | 111.4 | 1351.3 | | | | | | |
| | 8 | 22.4 | 885 | 2.4 | 12.2 | 112.2 | 724.9 | 22.6 | 1417 | 2.6 | 13.2 | 113.2 | 1257.1 |
| | 9 | | | | | | | | | | | | |
| | 10 | 22.8 | 1651 | 0.0 | 0.1 | 100.1 | 1487.1 | | | | | | |
| | Mean | 21.3 | 1366.1 | 1.7 | 9.1 | 109.1 | 1224.9 | 20.9 | 1236.8 | 2.1 | 11.0 | 111.0 | 1096.4 |
| | n | 9 | 9 | 9 | 9 | 9 | 9 | 4 | 4 | 4 | 4 | 4 | 4 |
| | SD | 2.3 | 531.1 | 0.9 | 4.8 | 4.8 | 532.4 | 3.1 | 274.0 | 0.6 | 2.9 | 2.9 | 276.3 |
| | SE | 0.8 | 177.0 | 0.3 | 1.6 | 1.6 | 177.5 | 1.6 | 137.0 | 0.3 | 1.5 | 1.5 | 138.2 |
| 4. OGX 20 ip qd*5, twk + | 1 | 23.0 | 447 | 1.2 | 5.3 | 105.3 | 327.1 | 23.0 | 308 | 1.2 | 5.4 | 105.4 | 188.2 |
| Velcade ® 0.5 mg/kg | 2 | 21.1 | 1254 | 2.2 | 11.8 | 111.8 | 1130.7 | 22.3 | 1937 | 3.5 | 18.3 | 118.3 | 1813.6 |
| iv biwk | 3 | 23.0 | 808 | 1.9 | 9.1 | 109.1 | 683.4 | 23.9 | 1385 | 2.8 | 13.3 | 113.3 | 1259.8 |
| | 4 | 21.6 | 743 | 2.0 | 9.9 | 109.9 | 609.6 | 22.6 | 1049 | 3.0 | 15.0 | 115.0 | 915.2 |
| | 5 | 22.0 | 2433 | 2.6 | 13.1 | 113.1 | 2290.0 | | | | | | |
| | 6 | 22.4 | 1488 | 3.3 | 17.5 | 117.5 | 1341.2 | 23.0 | 2509 | 4.0 | 20.9 | 120.9 | 2362.6 |
| | 7 | 20.9 | 448 | 1.7 | 8.9 | 108.9 | 297.5 | 22.1 | 738 | 2.9 | 15.1 | 115.1 | 587.8 |
| | 8 | 22.1 | 545 | 0.0 | 0.0 | 100.0 | 389.8 | 23.6 | 1234 | 1.5 | 6.8 | 106.8 | 1079.2 |
| | 9 | | | | | | | | | | | | |
| | 10 | 22.3 | 587 | 1.2 | 5.7 | 105.7 | 416.2 | 22.7 | 1061 | 1.6 | 7.7 | 107.7 | 890.0 |
| | Mean | 22.0 | 972.5 | 1.8 | 9.0 | 109.0 | 831.7 | 22.9 | 1277.6 | 2.5 | 12.8 | 112.8 | 1137.0 |
| | n | 9 | 9 | 9 | 9 | 9 | 9 | 8 | 8 | 8 | 8 | 8 | 8 |
| | SD | 0.7 | 655.8 | 0.9 | 5.1 | 5.1 | 657.2 | 0.6 | 686.1 | 1.0 | 5.6 | 5.6 | 685.6 |
| | SE | 0.2 | 218.6 | 0.3 | 1.7 | 1.7 | 219.1 | 0.2 | 242.6 | 0.4 | 2.0 | 2.0 | 242.4 |
| 5. OGX 40 ip qd*5, twk + | 1 | 21.4 | 360 | 1.4 | 6.8 | 106.8 | 237.0 | 21.3 | 384 | 1.3 | 6.3 | 106.3 | 261.2 |
| Velcade ® 0.5 mg/kg | 2 | 22.7 | 554 | 1.3 | 6.0 | 106.0 | 430.4 | 23.2 | 1220 | 1.8 | 8.3 | 108.3 | 1096.6 |
| iv biwk | 3 | 23.7 | 723 | 2.2 | 10.1 | 110.1 | 597.9 | 23.3 | 1500 | 1.8 | 8.5 | 108.5 | 1374.9 |
| | 4 | 21.1 | 214 | 1.1 | 5.4 | 105.4 | 79.9 | 20.6 | 253 | 0.5 | 2.7 | 102.7 | 118.5 |
| | 5 | | | | | | | | | | | | |
| | 6 | | | | | | | | | | | | |
| | 7 | 21.9 | 990 | 2.1 | 10.8 | 110.8 | 838.1 | 21.7 | 1783 | 2.0 | 10.2 | 110.2 | 1631.2 |
| | 8 | 22.5 | 543 | −0.5 | −2.3 | 97.7 | 385.9 | 21.8 | 565 | −1.3 | −5.6 | 94.4 | 408.0 |
| | 9 | 20.1 | 1076 | 0.4 | 2.2 | 102.2 | 914.4 | 20.2 | 1831 | 0.5 | 2.7 | 102.7 | 1669.9 |
| | 10 | 21.0 | 814 | 2.4 | 13.0 | 113.0 | 652.3 | 21.5 | 1377 | 2.9 | 15.3 | 115.3 | 1215.0 |
| | Mean | 21.8 | 659.2 | 1.3 | 6.5 | 106.5 | 517.0 | 21.7 | 1114.1 | 1.2 | 6.1 | 106.1 | 971.9 |
| | n | 8 | 8 | 8 | 8 | 8 | 8 | 8 | 8 | 8 | 8 | 8 | 8 |
| | SD | 1.1 | 298.3 | 1.0 | 5.0 | 5.0 | 287.8 | 1.1 | 628.7 | 1.3 | 6.2 | 6.2 | 622.2 |
| | SE | 0.4 | 105.5 | 0.4 | 1.8 | 1.8 | 101.8 | 0.4 | 222.3 | 0.4 | 2.2 | 2.2 | 220.0 |

### Example 15

### In Vivo Activity of Custersin (TV-1011) Against RPMI 8226 (Human Myeloma) Xenograft Model, Implanted Subcutaneously into Athymic Nude Mice

A previous in-house study demonstrated that custirsen in combination with a suboptimal dose concentration of Bortezomib had an inhibitory effect on tumor growth, but also showed unacceptable toxicity at a high dose.

In this study custirsen (TV-1011) is administered at different doses and regiments in combination with Bortezomib.

Materials and Methods

Test Articles
RPMI 8226 (Human Plasmacytoma, Myeloma B Cells) ATCC # CCL-155;
ECM Gel (Matrigel), Sigma-Aldrich, Cat # E1270, 5 ml;
RPMI (Beit Haemek);
Velcade® (Bortezomib) 3.5 mg lyophilized in vial; Mnf. 09-2011;
Custirsen (TV-1011) 20 mg/ml, K-46138;
Sodium chloride, TEVA.

Test Animals

120 CB.17 SCID female mice, 4-6 weeks old, 16-20 grams, obtained from Harlan animal breeding center.

Experimental Procedures

Cells Preparation

Cells (originated from ATCC) were cultured on RPMI medium. Cell suspension was centrifuged and resuspended in 50% Matrigel/HBSS to a final concentration of 7×10⁷ cells/ml. The suspension was implanted s.c. in the right flank of the anesthetized mouse at a volume of 100 μl.

Compounds Preparation

Velcade® was prepared once a week. Seven ml saline were added to the original vial containing 3.5 mg powder resulting in 0.5 mg/ml. Three ml of this solution were added to 27 ml saline to receive 0.05 mg/ml concentration. 11.2 ml of the stock solution of TV-1011 (20 mg/ml) were added to 44.8 ml saline to receive 4 mg/ml. 28.5 ml of 4 mg/ml were added to 9.5 saline to receive 3 mg/ml.

Experimental Design

Mice were implanted subcutaneously, with 7×10⁶ RPMI 8226 cells/mouse (in 50% Martigel/HBSS) on Day 0. On day 21, mice were sorted by the optimal average tumor volume (˜130 mm3) and were allocated into eight groups of 10 mice each.

| Gr. | N | Agent | Route | Dose & schedule |
|---|---|---|---|---|
| 1 | 10 | Vehicle | ip | qd*5, then biwk |
| 2 | 10 | Velcade ® | iv | 0.5 mg/kg biwk |
| 3 | 10 | Velcade ® + | iv | 0.5 mg/kg biwk |
| | | Custirsen (TV-1011) | ip | 30 mg/kg qd*5 |
| | | | | (loading only) |
| 4 | 10 | Velcade ® + | iv | 0.5 mg/kg biwk |
| | | Custirsen (TV-1011) | ip | 40 mg/kg twk, |
| | | | | then biwk |
| 5 | 10 | Velcade ® + | iv | 0.5 mg/kg biwk |
| | | Custirsen (TV-1011) | ip | 40 mg/kg twk, |
| | | | | then qwk |
| 6 | 10 | Velcade ® + | iv | 0.5 mg/kg biwk |
| | | Custirsen (TV-1011) | ip | 30 mg/kg qd*5, |
| | | | | then 40 mg/kg biwk |
| 7 | 10 | Velcade ® + | iv | 0.5 mg/kg biwk |
| | | Custirsen (TV-1011) | ip | 30 mg/kg qd*5, |
| | | | | then 30 mg/kg biwk |
| 8 | 10 | Custirsen (TV-1011) | ip | 30 qd*5, 40 biwk |

The treatment started on day 21 post implant, 0.2 ml per 20 grams v/w.

Statistical Analysis

Tumor volume was calculated as follows:

$$π \times {( \frac{width}{2} )}^{2} \times length .$$

The analysis of weight gain and tumor volume progression was made using one-way ANOVA followed by Tukey post-hoc comparisons.

Results

The treatment responses for Day 62 are summarized in Table 5.

All the treatments inhibited tumor growth compared to control group (Table 5), see also FIGS. 4 and 5. The effect of Velcade alone was moderate (43% TGI) and not statistically significant. Custirsen alone inhibited tumor growth by 67% (p<0.001). The addition of Velcade to Custirsen treatment (at the same doses and regimen) resulted in minor synergistic effect (72% TGI, p<0.001). The activity of all other combined treatments ranged between 43% TGI and 69% TGI with apparent advantage of maintained treatment over loading only.

TABLE 5
Summary of the results (day 62)

| | | | Maintenance | | Δ mean | | | | | | |
|---|---|---|---|---|---|---|---|---|---|---|---|
| | | Loading | Dose, | Total | tumor | | | | Max BW | | |
| | | Dose, | mg/kg/week* | dose, | volume, | | | No. of | (mean) | No. of | No. og |
| Compound | Regimen | mg/kg/week | 5 w | mg/kg | (mean ± se) | % TGI | No. of PR | CR | reduction | TRD | nTRD |
| 1. Vehicle | i.p. qd*5, then | | | | 1746 ± 218 | | 0 | 0 | −1.7% | 0 | 0 |
| | biwk | | | | | | | | day 28 | | |
| 2. Velcade ® | i.v. biwk | | | | 996 ± 179 | 43 | 0 | 0 | −0.5% | 0 | 0 |
| 0.5 mg/kg | | | | | | | | | day 28 | | |
| 3. Velcade ® + | i.v. biwk | | | | 861 ± 260 | 51* | 0 | 0 | −0.2% | 1 | |
| TV-1011 | i.p. qd*5 only | 150 | | 150 | | | | | day 33 | | |
| 4. Velcade ® + | i.v. biwk | | | | 535 ± 147 | 69*** | 1 | 0 | −0.6% | 0 | 0 |
| TV-1011 | twk in the 1- | 120 | 80 * 5 | 520 | | | | | day 33 | | |
| | st week, then | | | | | | | | | | |
| | biwk | | | | | | | | | | |
| 5. Velcade ® + | i.v. biwk | | | | 1003 ± 180 | 43 | 0 | 0 | — | 0 | 0 |
| TV-1011 | i.p. twk in the | 120 | 40 * 5 | 320 | | | | | | | |
| | 1-st week, | | | | | | | | | | |
| | then qwk | | | | | | | | | | |
| 6. Velcade ® + | i.v. biwk | | | | 496 ± 103 | 72*** | 1 | 0 | −3.2% | 0 | 1 |
| TV-1011 | i.p. qd*5, then | 150 | 80 * 5 | 550 | | | | | day 33 | | |
| | biwk | | | | | | | | | | |
| 7. Velcade ® + | i.v. biwk | | | | 742 ± 124 | 57** | 0 | 0 | −3.5% | 0 | 0 |
| TV-1011 | i.p. qd*5, then | 150 | 60 * 5 | 450 | | | | | day 33 | | |
| | biwk | | | | | | | | | | |
| 8. TV-1011 | i.p. qd*5, then | 150 | 80 * 5 | 550 | 574 ± 118 | 67*** | 1 | 0 | −0.5% | 0 | 0 |
| | biwk | | | | | | | | day 28 | | |

*p < 0.05,
**p < 0.01,
***p < 0.001 (one-way Anova, Tukey post-hoc test)

Conclusions
Custirsen 30 mg/kg qdx5 then 40 mg/kg biwk—alone was very efficacious; this regimen was not toxic;
The combination of Custirsen with Velcade was not much better than Custirsen alone;
The effect of continuous treatment with Custirsen is dose-dependent and results in better outcome versus loading only.

TABLE 6
Raw Data

| | | Day 21 | | Day 28 | | Δ | | % of | Day 33 | | | | % of | Day 41 | | | | % of |
|---|---|---|---|---|---|---|---|---|---|---|---|---|---|---|---|---|---|---|
| | | weight | volume | weight | volume | weight | Δ % | baseline | weight | volume | Δ weight | Δ % | baseline | weight | volume | Δ weight | Δ % | baseline |
| 1. Vehicle i.p. qd*5, then | 1 | 17.8 | 100 | 17.0 | 129 | −0.8 | −4.3 | 95.7 | 17.0 | 165 | −0.8 | −4.6 | 95.4 | 16.9 | 235 | −0.8 | −4.6 | 95.4 |
| biwk | 2 | 19.2 | 113 | 19.5 | 180 | 0.3 | 1.7 | 101.7 | 20.6 | 253 | 1.4 | 7.4 | 107.4 | 20.0 | 359 | 0.8 | 4.1 | 104.1 |
| | 3 | 21.1 | 116 | 21.6 | 244 | 0.4 | 2.1 | 102.1 | 21.1 | 270 | 0.0 | −0.2 | 99.8 | 21.1 | 368 | 0.0 | 0.1 | 100.1 |
| | 4 | 18.7 | 119 | 18.3 | 209 | −0.4 | −2.4 | 97.6 | 19.1 | 189 | 0.3 | 1.7 | 101.7 | 18.9 | 285 | 0.2 | 0.9 | 100.9 |
| | 5 | 18.1 | 124 | 17.0 | 199 | −1.1 | −6.1 | 93.9 | 17.4 | 274 | −0.6 | −3.6 | 96.4 | 18.1 | 336 | 0.1 | 0.3 | 100.3 |
| | 6 | 21.5 | 128 | 20.7 | 280 | −0.8 | −3.7 | 96.3 | 20.4 | 357 | −1.1 | −5.1 | 94.9 | 21.6 | 509 | 0.1 | 0.3 | 100.3 |
| | 7 | 20.3 | 132 | 20.1 | 183 | −0.2 | −0.9 | 99.1 | 20.0 | 171 | −0.2 | −1.2 | 98.8 | 20.3 | 481 | 0.1 | 0.3 | 100.3 |
| | 8 | 18.7 | 141 | 18.8 | 224 | 0.1 | 0.5 | 100.5 | 18.8 | 279 | 0.1 | 0.4 | 100.4 | 20.2 | 449 | 1.5 | 7.8 | 107.8 |
| | 9 | 19.1 | 154 | 19.1 | 277 | 0.0 | 0.1 | 100.1 | 18.9 | 334 | −0.2 | −1.2 | 98.8 | 19.4 | 607 | 0.3 | 1.5 | 101.5 |
| | 10 | 21.8 | 157 | 21.0 | 216 | −0.8 | −3.6 | 96.4 | 20.5 | 176 | −1.3 | −5.9 | 94.1 | 20.6 | 264 | −1.2 | −5.6 | 94.4 |
| | Mean | 19.6 | 128.5 | 19.3 | 214.1 | −0.3 | −1.7 | 98.3 | 19.4 | 246.9 | −0.3 | −1.2 | 98.8 | 19.7 | 389.5 | 0.1 | 0.5 | 100.5 |
| | n | 10 | 10 | 10 | 10 | 10 | 10 | 10 | 10 | 10 | 10 | 10 | 10 | 10 | 10 | 10 | 10 | 10 |
| | SD | 1.5 | 18.2 | 1.6 | 45.9 | 0.5 | 2.8 | 2.8 | 1.4 | 68.9 | 0.8 | 3.9 | 3.9 | 1.4 | 119.4 | 0.7 | 3.8 | 3.8 |
| | SE | 0.5 | 5.7 | 0.5 | 14.5 | 0.2 | 0.9 | 0.9 | 0.4 | 21.8 | 0.2 | 1.2 | 1.2 | 0.4 | 37.7 | 0.2 | 1.2 | 1.2 |
| 2. Velcade ® 0.5 mg/kg | 1 | 18.7 | 100 | 18.4 | 127 | −0.4 | −1.9 | 98.1 | 17.7 | 152 | −1.0 | −5.5 | 94.5 | 18.8 | 120 | 0.1 | 0.7 | 100.7 |
| biwk | 2 | 18.1 | 106 | 19.2 | 162 | 1.1 | 6.2 | 106.2 | 19.8 | 175 | 1.7 | 9.5 | 109.5 | 20.9 | 188 | 2.8 | 15.5 | 115.5 |
| | 3 | 19.0 | 117 | 19.4 | 223 | 0.5 | 2.6 | 102.6 | 19.4 | 216 | 0.5 | 2.6 | 102.6 | 20.2 | 288 | 1.2 | 6.4 | 106.4 |
| | 4 | 20.3 | 119 | 20.2 | 192 | −0.1 | −0.6 | 99.4 | 20.3 | 189 | 0.0 | 0.1 | 100.1 | 21.3 | 244 | 1.0 | 5.1 | 105.1 |
| | 5 | 19.7 | 124 | 19.2 | 155 | −0.4 | −2.2 | 97.8 | 20.0 | 187 | 0.3 | 1.7 | 101.7 | 20.8 | 225 | 1.1 | 5.5 | 105.5 |
| | 6 | 20.2 | 129 | 19.9 | 152 | −0.3 | −1.3 | 98.7 | 19.8 | 227 | −0.4 | −1.9 | 98.1 | 21.1 | 299 | 0.9 | 4.4 | 104.4 |
| | 7 | 18.6 | 133 | 17.9 | 172 | −0.7 | −3.7 | 96.3 | 18.0 | 165 | −0.6 | −3.4 | 96.6 | 18.7 | 191 | 0.1 | 0.6 | 100.6 |
| | 8 | 19.3 | 142 | 19.4 | 141 | 0.0 | 0.2 | 100.2 | 19.2 | 152 | −0.1 | −0.5 | 99.5 | 19.3 | 245 | 0.0 | 0.1 | 100.1 |
| | 9 | 20.8 | 154 | 20.4 | 243 | −0.4 | −1.9 | 98.1 | 20.9 | 225 | 0.1 | 0.5 | 100.5 | 21.4 | 280 | 0.6 | 2.8 | 102.8 |
| | 10 | 20.2 | 166 | 19.8 | 222 | −0.4 | −1.9 | 98.1 | 19.9 | 187 | −0.3 | −1.4 | 98.6 | 20.5 | 355 | 0.3 | 1.5 | 101.5 |
| | Mean | 19.5 | 129.3 | 19.4 | 178.9 | −0.1 | −0.5 | 99.5 | 19.5 | 187.6 | 0.0 | 0.2 | 100.2 | 20.3 | 243.6 | 0.8 | 4.3 | 104.3 |
| | n | 10 | 10 | 10 | 10 | 10 | 10 | 10 | 10 | 10 | 10 | 10 | 10 | 10 | 10 | 10 | 10 | 10 |
| | SD | 0.9 | 20.3 | 0.8 | 39.1 | 0.5 | 2.9 | 2.9 | 1.0 | 27.9 | 0.7 | 4.0 | 4.0 | 1.0 | 66.9 | 0.8 | 4.5 | 4.5 |
| | SE | 0.3 | 6.4 | 0.2 | 12.4 | 0.2 | 0.9 | 0.9 | 0.3 | 8.8 | 02 | 1.3 | 1.3 | 0.3 | 21.2 | 0.3 | 1.4 | 1.4 |
| 3. Velcade ® 0.5 mg/kg | 1 | 22.5 | 100 | 21.8 | 108 | −0.7 | −3.1 | 96.9 | 21.8 | 105 | −0.7 | −3.2 | 96.8 | 23.0 | 183 | 0.4 | 2.0 | 102.0 |
| iv biwk + TV-1011 | 2 | 19.8 | 108 | 20.3 | 124 | 0.5 | 2.4 | 102.4 | 20.5 | 119 | 0.7 | 3.7 | 103.7 | 21.3 | 206 | 1.5 | 7.5 | 107.5 |
| 30 mg/kg qd*5 | 3 | 15.8 | 114 | | | | | | | | | | | | | | | |
| | 4 | 20.6 | 120 | 21.1 | 175 | 0.4 | 2.1 | 102.1 | 20.5 | 174 | −0.1 | −0.4 | 99.6 | 21.9 | 224 | 1.2 | 6.0 | 106.0 |
| | 5 | 19.9 | 124 | 21.4 | 170 | 1.5 | 7.5 | 107.5 | 20.7 | 163 | 0.8 | 4.2 | 104.2 | 21.9 | 364 | 2.0 | 10.3 | 110.3 |
| | 6 | 20.2 | 129 | 20.1 | 145 | −0.1 | −0.4 | 99.6 | 19.0 | 128 | −1.2 | −5.9 | 94.1 | 20.6 | 139 | 0.4 | 2.2 | 102.2 |
| | 7 | 17.6 | 134 | 18.3 | 212 | 0.6 | 3.6 | 103.6 | 17.8 | 209 | 0.2 | 0.9 | 100.9 | 16.4 | 267 | 0.7 | 4.1 | 104.1 |
| | 8 | 15.7 | 142 | 16.0 | 145 | 0.3 | 2.0 | 102.0 | 15.5 | 206 | −0.2 | −1.2 | 98.8 | 16.7 | 279 | 0.9 | 6.0 | 106.0 |
| | 9 | 19.1 | 152 | 19.6 | 160 | 0.5 | 2.8 | 102.8 | 19.3 | 148 | 0.2 | 0.8 | 100.8 | 20.2 | 171 | 1.1 | 5.9 | 105.9 |
| | 10 | 20.8 | 165 | 20.8 | 203 | 0.0 | −0.1 | 99.9 | 20.7 | 227 | −0.2 | −0.8 | 99.2 | 21.6 | 290 | 0.7 | 3.5 | 103.5 |
| | Mean | 19.2 | 128.9 | 19.9 | 160.1 | 0.3 | 1.9 | 101.9 | 19.5 | 164.6 | −0.1 | −0.2 | 99.8 | 20.6 | 236.0 | 1.0 | 5.3 | 105.3 |
| | n | 10 | 10 | 9 | 9 | 9 | 9 | 9 | 9 | 9 | 9 | 9 | 9 | 9 | 9 | 9 | 9 | 9 |
| | SD | 2.2 | 20.2 | 1.8 | 34.3 | 0.6 | 3.0 | 3.0 | 1.9 | 43.1 | 0.6 | 3.2 | 3.2 | 2.0 | 70.4 | 0.5 | 2.6 | 2.6 |
| | SE | 0.7 | 6.4 | 0.6 | 11.4 | 0.2 | 1.0 | 1.0 | 0.6 | 14.4 | 0.2 | 1.1 | 1.1 | 0.7 | 23.5 | 0.2 | 0.9 | 0.9 |
| 4. Velcade ® 0.5 mg/kg | 1 | 15.5 | 104 | 16.6 | 197 | 1.1 | 7.0 | 107.0 | 16.3 | 184 | 0.8 | 4.8 | 104.8 | 15.2 | 162 | −0.3 | −2.0 | 98.0 |
| iv biwk + TV-1011 | 2 | 22.0 | 110 | 21.7 | 210 | −0.3 | −1.4 | 98.6 | 21.7 | 109 | −0.2 | −1.0 | 99.0 | 22.0 | 181 | 0.0 | 0.1 | 100.1 |
| 40 mg/kg twk, then biwk | 3 | 18.7 | 114 | 18.5 | 134 | −0.2 | −1.0 | 99.0 | 17.5 | 153 | −1.2 | −6.6 | 93.4 | 18.2 | 189 | −0.5 | −2.8 | 97.2 |
| | 4 | 21.2 | 118 | 21.2 | 155 | 0.1 | 0.3 | 100.3 | 20.1 | 171 | −1.0 | −4.8 | 95.2 | 20.6 | 165 | −0.6 | −2.8 | 97.2 |
| | 5 | 19.1 | 125 | 19.4 | 130 | 0.4 | 1.9 | 101.9 | 18.8 | 172 | −0.2 | −1.2 | 98.8 | 18.5 | 123 | −0.6 | −3.0 | 97.0 |
| | 6 | 21.8 | 130 | 21.6 | 122 | −0.2 | −1.1 | 98.9 | 21.4 | 139 | −0.4 | −1.7 | 98.3 | 21.8 | 149 | 0.0 | 0.2 | 100.2 |
| | 7 | 18.1 | 134 | 18.5 | 167 | 0.5 | 2.6 | 102.6 | 17.8 | 189 | −0.2 | −1.2 | 98.8 | 18.6 | 160 | 0.6 | 3.1 | 103.1 |
| | 8 | 21.2 | 144 | 22.0 | 124 | 0.7 | 3.5 | 103.5 | 21.2 | 230 | 0.0 | −0.1 | 99.9 | 21.2 | 168 | −0.1 | −0.3 | 99.7 |
| | 9 | 18.7 | 152 | 19.5 | 137 | 0.8 | 4.1 | 104.1 | 19.8 | 196 | 1.1 | 5.6 | 105.6 | 19.7 | 182 | 1.0 | 5.3 | 105.3 |
| | 10 | 19.5 | 167 | 19.5 | 192 | 0.0 | 0.1 | 100.1 | 19.5 | 216 | 0.0 | −0.2 | 99.8 | 19.4 | 146 | −0.1 | −0.6 | 99.4 |
| | Mean | 19.6 | 129.7 | 19.8 | 156.7 | 0.3 | 1.6 | 101.6 | 19.4 | 175.9 | −0.2 | −0.6 | 99.4 | 19.5 | 162.3 | −0.1 | −0.3 | 99.7 |
| | n | 10 | 10 | 10 | 10 | 10 | 10 | 10 | 10 | 10 | 10 | 10 | 10 | 10 | 10 | 10 | 10 | 10 |
| | SD | 2.0 | 19.9 | 1.7 | 32.8 | 0.5 | 2.7 | 2.7 | 1.8 | 36.0 | 0.7 | 3.7 | 3.7 | 2.0 | 19.7 | 0.5 | 2.7 | 2.7 |
| | SE | 0.6 | 6.3 | 0.5 | 10.4 | 0.2 | 0.9 | 0.9 | 0.6 | 11.4 | 0.2 | 1.2 | 1.2 | 0.6 | 6.2 | 0.2 | 0.9 | 0.9 |
| 5. Velcade ® 0.5 mg/kg | 1 | 19.7 | 104 | 19.6 | 84 | −0.1 | −0.6 | 99.4 | 20.0 | 129 | 0.3 | 1.4 | 101.4 | 21.0 | 190 | 1.3 | 6.6 | 106.6 |
| iv biwk + TV-1011 | 2 | 17.7 | 111 | 18.3 | 163 | 0.7 | 3.7 | 103.7 | 18.0 | 137 | 0.4 | 2.1 | 102.1 | 20.0 | 174 | 2.4 | 13.3 | 113.3 |
| 40 mg/kg twk, then qwk | 3 | 19.8 | 114 | 20.2 | 173 | 0.4 | 2.2 | 102.2 | 19.7 | 150 | −0.1 | 0.6 | 99.4 | 20.8 | 177 | 1.0 | 5.0 | 105.0 |
| | 4 | 17.6 | 118 | 17.9 | 183 | 0.3 | 1.4 | 101.4 | 18.7 | 154 | 1.1 | 6.0 | 106.0 | 19.9 | 257 | 2.3 | 12.8 | 112.8 |
| | 5 | 16.7 | 120 | 16.1 | 107 | −0.6 | −3.4 | 96.6 | 16.0 | 96 | −0.7 | −4.1 | 95.9 | 17.3 | 138 | 0.6 | 3.8 | 103.8 |
| | 6 | 20.2 | 130 | 20.5 | 166 | 0.3 | 1.4 | 101.4 | 20.5 | 122 | 0.3 | 1.4 | 101.4 | 21.2 | 238 | 1.0 | 4.9 | 104.9 |
| | 7 | 20.0 | 135 | 20.1 | 202 | 0.1 | 0.6 | 100.6 | 20.3 | 186 | 0.3 | 1.6 | 101.6 | 21.5 | 261 | 1.5 | 7.4 | 107.4 |
| | 8 | 16.1 | 145 | 16.3 | 147 | 0.3 | 1.6 | 101.6 | 17.6 | 166 | 1.6 | 9.7 | 109.7 | 19.0 | 227 | 2.9 | 18.0 | 118.0 |
| | 9 | 19.1 | 151 | 19.3 | 170 | 0.2 | 1.1 | 101.1 | 19.5 | 149 | 0.5 | 2.5 | 102.5 | 20.6 | 172 | 1.5 | 8.0 | 108.0 |
| | 10 | 18.2 | 165 | 18.4 | 196 | 0.2 | 1.3 | 101.3 | 17.8 | 176 | −0.4 | −2.0 | 98.0 | 19.3 | 143 | 1.1 | 5.9 | 105.9 |
| | Mean | 18.5 | 129.3 | 18.7 | 159.0 | 0.2 | 0.9 | 100.9 | 18.8 | 146.5 | 0.3 | 1.8 | 101.8 | 20.0 | 197.6 | 1.5 | 8.5 | 108.6 |
| | n | 10 | 10 | 10 | 10 | 10 | 10 | 10 | 10 | 10 | 10 | 10 | 10 | 10 | 10 | 10 | 10 | 10 |
| | SD | 1.5 | 19.6 | 1.6 | 37.6 | 0.3 | 1.9 | 1.9 | 1.4 | 26.7 | 0.6 | 3.9 | 3.9 | 1.3 | 45.0 | 0.7 | 4.6 | 4.6 |
| | SE | 0.5 | 6.2 | 0.5 | 11.9 | 0.1 | 0.6 | 0.6 | 0.5 | 8.4 | 0.2 | 1.2 | 1.2 | 0.4 | 14.2 | 0.2 | 1.5 | 1.5 |
| 6. Velcade ® 0.5 mg/kg | 1 | 16.3 | 105 | 16.0 | 134 | −0.3 | −1.8 | 98.4 | 14.6 | 113 | −1.7 | −10.6 | 89.4 | 15.1 | 109 | −1.2 | −7.4 | 92.6 |
| iv biwk + TV-1011 | 2 | 19.6 | 112 | 19.7 | 135 | 0.0 | 0.2 | 100.2 | 19.5 | 103 | −0.2 | −0.9 | 99.1 | 20.3 | 112 | 0.7 | 3.6 | 103.6 |
| 30 mg/kg qd*5, then | 3 | 18.5 | 115 | 18.9 | 103 | 0.4 | 2.4 | 102.4 | 19.1 | 99 | 0.6 | 3.2 | 103.2 | 20.4 | 148 | 2.0 | 10.6 | 110.6 |
| 40 mg/kg biwk | 4 | 21.0 | 119 | 21.1 | 166 | 0.1 | 0.4 | 100.4 | 20.3 | 141 | −0.7 | −3.1 | 96.9 | 19.9 | 82 | −1.1 | −5.3 | 94.7 |
| | 5 | 19.8 | 120 | 19.1 | 167 | −0.6 | −3.2 | 96.8 | 19.4 | 146 | −0.3 | −1.7 | 98.3 | 20.7 | 140 | 0.9 | 4.8 | 104.8 |
| | 6 | 21.7 | 125 | 21.0 | 172 | −0.6 | −2.9 | 97.1 | 20.7 | 123 | −1.0 | −4.5 | 95.5 | 20.8 | 143 | −0.8 | −3.9 | 96.1 |
| | 7 | 20.2 | 137 | 19.7 | 201 | −0.5 | −2.5 | 97.5 | 19.5 | 184 | −0.7 | −3.4 | 96.6 | 21.2 | 207 | 1.1 | 5.4 | 105.4 |
| | 8 | 18.9 | 145 | 18.5 | 184 | −0.5 | −2.6 | 97.4 | 18.5 | 117 | −0.5 | −2.6 | 97.4 | 20.1 | 128 | 1.2 | 6.1 | 106.1 |
| | 9 | 19.6 | 150 | 19.0 | 157 | −0.6 | −3.0 | 97.0 | 18.5 | 105 | −1.1 | −5.8 | 94.2 | 18.9 | 86 | −0.7 | −3.7 | 96.3 |
| | 10 | 19.0 | 170 | 19.4 | 165 | 0.4 | 2.4 | 102.4 | 18.4 | 174 | −0.5 | −2.8 | 97.2 | 19.6 | 158 | 0.7 | 3.6 | 103.6 |
| | Mean | 19.4 | 129.7 | 19.2 | 158.3 | −0.2 | −1.0 | 99.0 | 18.8 | 130.6 | −0.6 | −3.2 | 96.8 | 19.7 | 131.4 | 0.3 | 1.4 | 101.4 |
| | n | 10 | 10 | 10 | 10 | 10 | 10 | 10 | 10 | 10 | 10 | 10 | 10 | 10 | 10 | 10 | 10 | 10 |
| | SD | 1.5 | 20.4 | 1.4 | 28.0 | 0.4 | 2.2 | 2.2 | 1.7 | 29.9 | 0.6 | 3.5 | 3.5 | 1.8 | 36.9 | 1.1 | 5.9 | 5.9 |
| | SE | 0.5 | 6.5 | 0.4 | 8.9 | 0.1 | 0.7 | 0.7 | 0.5 | 9.5 | 0.2 | 1.1 | 1.1 | 0.6 | 11.7 | 0.4 | 1.9 | 1.9 |
| 7. Velcade ® 0.5 mg/kg | 1 | 21.2 | 106 | 19.9 | 128 | −1.3 | −6.2 | 93.8 | 19.9 | 188 | −1.3 | −6.3 | 93.7 | 20.5 | 190 | −0.7 | −3.5 | 96.5 |
| iv biwk + TV-1011 | 2 | 20.8 | 112 | 21.0 | 131 | 0.2 | 1.0 | 101.0 | 20.3 | 121 | −0.5 | −2.5 | 97.5 | 21.2 | 146 | 0.3 | 1.7 | 101.7 |
| 30 mg/kg qd*5, then | 3 | 18.4 | 115 | 18.1 | 141 | −0.3 | −1.4 | 98.6 | 18.8 | 149 | 0.5 | 2.7 | 102.7 | 20.1 | 122 | 1.7 | 9.5 | 109.5 |
| 30 mg/kg biwk | 4 | 18.5 | 119 | 18.6 | 220 | 0.2 | 0.9 | 100.9 | 17.6 | 187 | −0.8 | −4.5 | 95.5 | 18.0 | 163 | −0.5 | −2.7 | 97.3 |
| | 5 | 18.1 | 121 | 18.6 | 202 | 0.5 | 2.8 | 102.8 | 17.7 | 137 | −0.3 | −1.9 | 98.1 | 19.4 | 111 | 1.4 | 7.6 | 107.6 |
| | 6 | 18.3 | 127 | 16.9 | 131 | −1.4 | −7.8 | 92.2 | 17.1 | 173 | −1.2 | −6.6 | 93.4 | 17.4 | 196 | −0.9 | −5.1 | 94.9 |
| | 7 | 20.4 | 130 | 20.2 | 173 | −0.2 | −0.9 | 99.1 | 18.8 | 151 | −1.5 | −7.5 | 92.5 | 19.5 | 164 | −0.8 | −4.0 | 96.0 |
| | 8 | 20.1 | 147 | 19.7 | 197 | −0.4 | −2.1 | 97.9 | 19.2 | 128 | −0.9 | −4.4 | 95.6 | 19.9 | 113 | −0.2 | −1.0 | 99.0 |
| | 9 | 18.8 | 148 | 18.4 | 167 | −0.4 | −2.1 | 97.9 | 19.0 | 171 | 0.2 | 1.1 | 101.1 | 19.6 | 220 | 0.9 | 4.5 | 104.5 |
| | 10 | 19.1 | 166 | 18.6 | 242 | −0.5 | −2.7 | 97.3 | 18.1 | 231 | −1.1 | −5.5 | 94.5 | 19.4 | 281 | 0.3 | 1.7 | 101.7 |
| | Mean | 19.4 | 129.1 | 19.0 | 173.1 | −0.4 | −1.9 | 98.1 | 18.7 | 163.7 | −0.7 | −3.5 | 96.5 | 19.5 | 170.6 | 0.1 | 0.9 | 100.9 |
| | n | 10 | 10 | 10 | 10 | 10 | 10 | 10 | 10 | 10 | 10 | 10 | 10 | 10 | 10 | 10 | 10 | 10 |
| | SD | 1.2 | 18.8 | 1.2 | 40.8 | 0.6 | 3.2 | 3.2 | 1.0 | 33.3 | 0.7 | 3.4 | 3.4 | 1.1 | 53.3 | 0.9 | 5.0 | 5.0 |
| | SE | 0.4 | 6.0 | 0.4 | 12.9 | 0.2 | 1.0 | 1.0 | 0.3 | 10.5 | 0.2 | 1.1 | 1.1 | 0.4 | 16.9 | 0.3 | 1.6 | 1.6 |
| 8. TV-1011 30 mg/kg | 1 | 21.4 | 107 | 21.0 | 78 | −0.4 | −2.0 | 98.0 | 20.9 | 132 | −0.5 | −2.2 | 97.8 | 21.7 | 76 | 0.3 | 1.4 | 101.4 |
| qd*5, 40 mg/kg biwk | 2 | 17.7 | 113 | 118.5 | 127 | 0.8 | 4.4 | 104.4 | 19.2 | 131 | 1.5 | 8.3 | 108.3 | 20.0 | 144 | 2.3 | 12.9 | 112.9 |
| | 3 | 20.5 | 116 | 21.3 | 114 | 0.8 | 4.0 | 104.0 | 22.1 | 120 | 1.7 | 8.1 | 108.1 | 22.8 | 140 | 2.3 | 11.2 | 111.2 |
| | 4 | 15.9 | 119 | 16.3 | 149 | 0.4 | 2.6 | 102.6 | 15.2 | 116 | −0.6 | −4.1 | 95.9 | 17.2 | 139 | 1.3 | 8.4 | 108.4 |
| | 5 | 19.6 | 123 | 18.7 | 180 | −0.9 | −4.8 | 95.2 | 18.4 | 167 | −1.2 | −6.0 | 94.0 | 20.2 | 336 | 0.6 | 2.9 | 102.9 |
| | 6 | 20.3 | 128 | 19.6 | 204 | −0.6 | −3.1 | 96.9 | 20.5 | 157 | 0.3 | 1.3 | 101.3 | 20.7 | 249 | 0.5 | 2.3 | 102.3 |
| | 7 | 17.9 | 131 | 16.9 | 159 | −1.0 | 5.6 | 94.4 | 17.6 | 100 | −0.3 | −1.7 | 98.3 | 18.9 | 152 | 1.1 | 5.9 | 105.9 |
| | 8 | 16.6 | 138 | 17.1 | 175 | 0.5 | 3.0 | 103.0 | 17.5 | 154 | 0.9 | 5.5 | 105.5 | 18.7 | 185 | 2.0 | 12.3 | 112.3 |
| | 9 | 20.1 | 147 | 19.9 | 193 | −0.3 | −1.3 | 98.7 | 20.1 | 206 | 0.0 | −0.1 | 99.9 | 21.5 | 223 | 1.3 | 6.7 | 106.7 |
| | 10 | 17.8 | 172 | 17.5 | 204 | −0.3 | −1.7 | 98.3 | 19.1 | 186 | 1.3 | 7.3 | 107.3 | 18.5 | 200 | 0.7 | 3.9 | 103.9 |
| | Mean | 18.8 | 129.3 | 18.7 | 158.1 | −0.1 | −0.5 | 99.5 | 19.1 | 147.0 | 0.3 | 1.6 | 101.6 | 20.0 | 184.3 | 1.2 | 6.8 | 106.8 |
| | n | 10 | 10 | 10 | 10 | 10 | 10 | 10 | 10 | 10 | 10 | 10 | 10 | 10 | 10 | 10 | 10 | 10 |
| | SD | 1.8 | 19.2 | 1.7 | 41.7 | 0.7 | 3.7 | 3.7 | 2.0 | 33.3 | 1.0 | 5.3 | 5.3 | 1.7 | 72.4 | 0.8 | 4.3 | 4.3 |
| | SE | 0.6 | 6.1 | 0.6 | 13.2 | 0.2 | 1.2 | 1.2 | 0.6 | 10.5 | 0.3 | 1.7 | 1.7 | 0.5 | 22.9 | 0.2 | 1.3 | 1.3 |

| | | Day 48 | | | | % of | Day 55 | | | | % of | Day 62 | | | | % of | |
|---|---|---|---|---|---|---|---|---|---|---|---|---|---|---|---|---|---|
| | | weight | volume | Δ weight | Δ % | baseline | weight | volume | Δ weight | Δ % | baseline | weight | volume | Δ weight | Δ % | baseline | Δ volume |
| 1. Vehicle i.p. qd*5, then | 1 | 16.8 | 341 | −1.0 | −5.5 | 94.5 | 17.4 | 540 | −0.4 | −2.3 | 97.7 | 17.8 | 1006 | 0.0 | 0.3 | 100.3 | 905.5 |
| biwk | 2 | 21.7 | 514 | 2.6 | 13.4 | 113.4 | 21.8 | 1417 | 2.6 | 13.7 | 113.7 | 22.9 | 2026 | 3.7 | 19.3 | 119.3 | 1913.4 |
| | 3 | 21.6 | 460 | 0.5 | 2.2 | 102.2 | 22.0 | 900 | 0.8 | 4.0 | 104.0 | 22.2 | 1554 | 1.1 | 5.0 | 106.0 | 1437.3 |
| | 4 | 19.5 | 674 | 0.7 | 4.0 | 104.0 | 21.4 | 1242 | 2.6 | 14.1 | 114.1 | 21.1 | 2256 | 2.4 | 12.7 | 112.7 | 2137.4 |
| | 5 | 18.2 | 384 | 0.1 | 0.6 | 100.6 | 19.6 | 544 | 1.6 | 8.6 | 108.6 | 19.0 | 963 | 0.9 | 4.8 | 104.8 | 829.0 |
| | 6 | 22.3 | 813 | 0.8 | 3.6 | 103.6 | 23.0 | 1495 | 1.5 | 6.9 | 106.9 | 23.7 | 2418 | 2.2 | 10.0 | 110.0 | 2289.3 |
| | 7 | 20.5 | 726 | 0.3 | 1.3 | 101.3 | 22.0 | 1100 | 1.7 | 8.4 | 108.4 | 22.1 | 2252 | 1.8 | 8.9 | 108.9 | 2119.9 |
| | 8 | 19.6 | 755 | 0.8 | 4.5 | 104.5 | 21.1 | 1260 | 2.4 | 12.7 | 112.7 | 22.2 | 2291 | 3.5 | 18.8 | 118.8 | 2150.2 |
| | 9 | 20.1 | 1104 | 1.1 | 5.5 | 105.5 | 20.9 | 1667 | 1.8 | 9.3 | 109.3 | 22.4 | 2945 | 3.3 | 17.1 | 117.1 | 2790.8 |
| | 10 | 21.2 | 410 | −0.6 | −2.9 | 97.1 | 22.3 | 599 | 0.5 | 2.2 | 102.2 | 23.0 | 1040 | 1.2 | 5.4 | 105.4 | 882.7 |
| | Mean | 20.1 | 618.0 | 0.5 | 2.7 | 102.7 | 21.1 | 1076.5 | 1.5 | 7.8 | 107.8 | 21.6 | 1874.0 | 2.0 | 10.2 | 110.2 | 1745.6 |
| | n | 10 | 10 | 10 | 10 | 10 | 10 | 10 | 10 | 10 | 10 | 10 | 10 | 10 | 10 | 10 | 10 |
| | SD | 1.7 | 240.0 | 1.0 | 5.1 | 5.1 | 1.6 | 412.5 | 1.0 | 5.3 | 5.3 | 1.9 | 693.6 | 1.2 | 6.6 | 6.6 | 687.9 |
| | SE | 0.5 | 75.9 | 0.3 | 1.6 | 1.6 | 0.5 | 130.4 | 0.3 | 1.7 | 1.7 | 0.6 | 219.3 | 0.4 | 2.1 | 2.1 | 217.5 |
| 2. Velcade ® 0.5 mg/kg | 1 | 19.1 | 180 | 0.4 | 1.9 | 101.9 | 20.3 | 164 | 1.6 | 8.6 | 108.6 | 19.9 | 174 | 1.2 | 6.3 | 106.3 | 74.3 |
| biwk | 2 | 18.9 | 423 | 1.8 | 9.9 | 109.9 | 21.4 | 924 | 3.3 | 18.2 | 118.2 | 21.9 | 1747 | 3.6 | 21.2 | 121.2 | 1638.7 |
| | 3 | 22.0 | 682 | 3.1 | 16.1 | 116.1 | 22.4 | 1082 | 3.5 | 18.4 | 118.4 | 23.1 | 1988 | 4.2 | 22.1 | 122.1 | 1870.6 |
| | 4 | 21.3 | 441 | 1.0 | 5.1 | 105.1 | 21.8 | 644 | 1.5 | 7.1 | 107.1 | 22.4 | 1094 | 2.1 | 10.4 | 110.4 | 974.8 |
| | 5 | 20.9 | 282 | 1.2 | 6.3 | 106.3 | 21.6 | 403 | 1.9 | 9.6 | 109.6 | 21.0 | 726 | 1.3 | 6.7 | 106.7 | 601.2 |
| | 6 | 21.3 | 431 | 1.2 | 5.7 | 105.7 | 21.6 | 809 | 1.6 | 8.1 | 108.1 | 23.1 | 1451 | 2.9 | 14.3 | 114.3 | 1322.3 |
| | 7 | 18.4 | 252 | 0.8 | 4.2 | 104.2 | 19.9 | 562 | 1.4 | 7.3 | 107.3 | 20.2 | 1084 | 1.7 | 8.9 | 108.9 | 950.8 |
| | 8 | 20.2 | 281 | 0.9 | 4.6 | 104.6 | 20.8 | 481 | 1.5 | 7.8 | 107.8 | 21.9 | 924 | 2.6 | 13.2 | 113.2 | 782.5 |
| | 9 | 22.5 | 363 | 1.7 | 8.0 | 106.0 | 22.7 | 371 | 1.9 | 9.1 | 109.1 | 23.2 | 520 | 2.4 | 11.3 | 111.3 | 366.6 |
| | 10 | 20.9 | 490 | 0.8 | 3.8 | 103.8 | 22.3 | 894 | 2.2 | 10.9 | 110.9 | 23.0 | 1544 | 2.9 | 14.3 | 114.3 | 1377.6 |
| | Mean | 20.8 | 382.6 | 1.3 | 6.6 | 106.6 | 21.5 | 633.5 | 2.0 | 10.5 | 110.5 | 22.0 | 1125.2 | 2.5 | 12.9 | 112.9 | 995.9 |
| | n | 10 | 10 | 10 | 10 | 10 | 10 | 10 | 10 | 10 | 10 | 10 | 10 | 10 | 10 | 10 | 10 |
| | SD | 1.1 | 144.3 | 0.8 | 4.0 | 4.0 | 0.9 | 289.6 | 08 | 4.3 | 4.3 | 1.2 | 566.2 | 1.0 | 5.4 | 5.4 | 565.7 |
| | SE | 0.4 | 45.6 | 0.2 | 1.3 | 1.3 | 0.3 | 91.6 | 0.2 | 1.3 | 1.3 | 0.4 | 179.0 | 0.3 | 1.7 | 1.7 | 178.9 |
| 3. Velcade ® 0.5 mg/kg | 1 | 24.2 | 374 | 1.7 | 7.4 | 107.4 | 24.4 | 619 | 1.9 | 8.4 | 108.4 | 24.9 | 1178 | 2.4 | 10.6 | 110.6 | 1078.0 |
| iv biwk + TV-1011 | 2 | 21.8 | 253 | 2.0 | 10.1 | 110.1 | 22.8 | 469 | 3.1 | 15.4 | 115.4 | 22.8 | 692 | 3.0 | 15.1 | 115.1 | 584.4 |
| 30 mg/kg qd*5 | 3 | | | | | | | | | | | | | | | | |
| | 4 | 21.6 | 211 | 0.9 | 4.6 | 104.6 | 22.4 | 303 | 1.8 | 8.6 | 108.6 | 22.1 | 405 | 1.5 | 7.0 | 107.0 | 285.3 |
| | 5 | 22.2 | 859 | 2.3 | 11.7 | 111.7 | 23.4 | 1442 | 3.8 | 17.9 | 117.9 | 23.6 | 2815 | 3.7 | 18.8 | 118.8 | 2690.7 |
| | 6 | 21.6 | 132 | 1.5 | 7.3 | 107.3 | 21.6 | 253 | 1.4 | 6.9 | 106.9 | 21.3 | 273 | 1.2 | 5.8 | 105.8 | 144.0 |
| | 7 | 19.3 | 458 | 1.7 | 9.5 | 109.5 | 19.9 | 802 | 2.2 | 12.6 | 112.6 | 20.2 | 1116 | 2.5 | 14.3 | 114.3 | 982.3 |
| | 8 | 17.1 | 474 | 1.4 | 8.9 | 108.9 | 16.8 | 652 | 1.1 | 7.1 | 107.1 | 18.0 | 982 | 2.3 | 14.6 | 114.6 | 640.0 |
| | 9 | 22.1 | 244 | 3.0 | 15.7 | 115.7 | 21.7 | 272 | 2.6 | 13.7 | 113.7 | 22.2 | 293 | 3.1 | 16.3 | 116.3 | 140.8 |
| | 10 | 22.5 | 447 | 1.6 | 7.9 | 107.9 | 23.0 | 457 | 2.2 | 10.4 | 110.4 | 23.3 | 1169 | 2.4 | 11.7 | 111.7 | 1003.7 |
| | Mean | 21.4 | 383.6 | 1.8 | 9.2 | 109.2 | 21.8 | 585.5 | 2.2 | 11.2 | 111.2 | 22.0 | 991.6 | 2.5 | 12.7 | 112.7 | 861.0 |
| | n | 9 | 9 | 9 | 9 | 9 | 9 | 9 | 9 | 9 | 9 | 9 | 9 | 9 | 9 | 9 | 9 |
| | SD | 2.0 | 215.9 | 0.6 | 3.1 | 3.1 | 2.3 | 371.6 | 0.8 | 3.9 | 3.9 | 2.0 | 778.2 | 0.8 | 4.3 | 4.3 | 780.3 |
| | SE | 0.7 | 72.0 | 0.2 | 1.0 | 1.0 | 0.8 | 123.9 | 0.3 | 1.3 | 1.3 | 0.7 | 259.4 | 0.3 | 1.4 | 1.4 | 260.1 |
| 4. Velcade ® 0.5 mg/kg | 1 | 16.7 | 501 | 1.2 | 7.4 | 107.4 | 17.0 | 524 | 1.5 | 9.4 | 109.4 | 16.7 | 1007 | 1.2 | 7.9 | 107.9 | 902.8 |
| iv biwk + TV-1011 | 2 | 23.7 | 246 | 1.7 | 7.9 | 107.9 | 23.8 | 610 | 1.8 | 8.1 | 108.1 | 24.6 | 987 | 2.7 | 12.2 | 112.2 | 877.0 |
| 40 mg/kg twk, then biwk | 3 | 20.3 | 321 | 1.6 | 8.4 | 108.4 | 20.7 | 473 | 1.9 | 10.3 | 110.3 | 19.7 | 1055 | 1.0 | 5.1 | 105.1 | 941.4 |
| | 4 | 22.4 | 511 | 1.2 | 5.6 | 105.6 | 23.2 | 676 | 2.1 | 9.8 | 109.8 | 22.1 | 1218 | 1.0 | 4.8 | 104.6 | 1099.8 |
| | 5 | 21.1 | 161 | 2.1 | 10.8 | 110.8 | 21.1 | 178 | 2.0 | 10.7 | 110.7 | 20.2 | 167 | 1.2 | 6.1 | 106.1 | 42.9 |
| | 6 | 23.6 | 191 | 1.8 | 6.4 | 106.4 | 23.9 | 392 | 2.1 | 9.6 | 109.6 | 22.3 | 621 | 0.5 | 2.4 | 102.4 | 491.6 |
| | 7 | 19.8 | 196 | 1.8 | 9.8 | 109.8 | 19.8 | 187 | 1.7 | 9.4 | 109.4 | 19.5 | 199 | 1.4 | 7.8 | 107.6 | 64.2 |
| | 8 | 23.3 | 209 | 2.0 | 9.6 | 109.6 | 23.2 | 120 | 2.0 | 9.3 | 109.3 | 22.8 | 83 | 1.6 | 7.5 | 107.5 | −60.6 |
| | 9 | 22.3 | 358 | 3.6 | 19.1 | 119.1 | 22.3 | 496 | 3.6 | 19.2 | 119.2 | 22.7 | 1083 | 4.0 | 21.5 | 121.5 | 931.2 |
| | 10 | 21.0 | 197 | 1.6 | 8.0 | 108.0 | 21.2 | 248 | 1.7 | 8.8 | 108.8 | 21.5 | 227 | 2.0 | 10.1 | 110.1 | 59.8 |
| | Mean | 21.4 | 289.1 | 1.8 | 9.5 | 109.5 | 21.6 | 380.3 | 2.0 | 10.5 | 110.5 | 21.2 | 664.7 | 1.6 | 8.5 | 108.5 | 535.0 |
| | n | 10 | 10 | 10 | 10 | 10 | 10 | 10 | 10 | 10 | 10 | 10 | 10 | 10 | 10 | 10 | 10 |
| | SD | 2.2 | 129.6 | 0.7 | 3.7 | 3.7 | 2.2 | 185.8 | 0.6 | 3.2 | 3.2 | 2.2 | 453.4 | 1.0 | 5.3 | 5.3 | 464.2 |
| | SE | 0.7 | 41.0 | 0.2 | 1.2 | 1.2 | 0.7 | 58.8 | 0.2 | 1.0 | 1.0 | 0.7 | 143.4 | 0.3 | 1.7 | 1.7 | 146.8 |
| 5. Velcade ® 0.5 mg/kg | 1 | 22.3 | 331 | 2.6 | 13.2 | 113.2 | 21.5 | 489 | 1.8 | 9.0 | 109.0 | 21.9 | 797 | 2.2 | 11.4 | 111.4 | 693.3 |
| iv biwk + TV-1011 | 2 | 21.0 | 199 | 3.3 | 18.9 | 118.9 | 20.6 | 260 | 2.9 | 16.5 | 116.5 | 20.1 | 318 | 2.5 | 13.9 | 113.9 | 207.1 |
| 40 mg/kg twk, then qwk | 3 | 22.7 | 287 | 2.9 | 14.4 | 114.4 | 21.8 | 388 | 2.0 | 9.9 | 109.9 | 21.8 | 670 | 2.0 | 10.2 | 110.2 | 556.5 |
| | 4 | 21.3 | 586 | 3.7 | 21.3 | 121.3 | 21.8 | 758 | 4.2 | 23.6 | 123.6 | 22.3 | 1735 | 4.7 | 26.9 | 126.9 | 1617.0 |
| | 5 | 18.7 | 216 | 2.0 | 11.8 | 111.8 | 18.7 | 262 | 2.0 | 11.7 | 111.7 | 19.0 | 519 | 2.2 | 13.4 | 113.4 | 399.3 |
| | 6 | 22.8 | 505 | 2.6 | 12.7 | 112.7 | 22.6 | 821 | 2.4 | 11.8 | 111.8 | 23.9 | 1296 | 3.7 | 18.1 | 118.1 | 1167.8 |
| | 7 | 22.8 | 619 | 2.6 | 13.1 | 113.1 | 22.9 | 1186 | 2.9 | 14.6 | 114.6 | 23.2 | 2187 | 3.2 | 15.9 | 115.9 | 2052.5 |
| | 8 | 20.0 | 633 | 4.0 | 24.7 | 124.7 | 20.1 | 760 | 4.1 | 25.4 | 125.4 | 20.0 | 1293 | 4.0 | 24.6 | 124.6 | 1148.0 |
| | 9 | 22.5 | 362 | 3.5 | 18.2 | 118.2 | 22.7 | 618 | 3.7 | 19.3 | 119.3 | 22.5 | 1436 | 3.5 | 18.1 | 118.1 | 1285.3 |
| | 10 | 20.8 | 345 | 2.6 | 14.5 | 114.5 | 20.9 | 425 | 2.7 | 14.9 | 114.9 | 21.0 | 1067 | 2.8 | 15.4 | 115.4 | 901.6 |
| | Mean | 21.5 | 408.2 | 3.0 | 16.3 | 116.3 | 21.4 | 596.8 | 2.9 | 15.7 | 115.7 | 21.6 | 1132.2 | 3.1 | 16.8 | 116.8 | 1002.8 |
| | n | 10 | 10 | 10 | 10 | 10 | 10 | 10 | 10 | 10 | 10 | 10 | 10 | 10 | 10 | 10 | 10 |
| | SD | 1.4 | 164.4 | 0.6 | 4.3 | 4.3 | 1.3 | 290.6 | 0.9 | 5.6 | 5.6 | 1.5 | 576.3 | 0.9 | 5.4 | 5.4 | 568.6 |
| | SE | 0.4 | 52.0 | 0.2 | 1.4 | 1.4 | 0.4 | 91.9 | 0.3 | 1.8 | 1.8 | 0.5 | 182.3 | 0.3 | 1.7 | 1.7 | 179.8 |
| 6. Velcade ® 0.5 mg/kg | 1 | 16.6 | 180 | 0.3 | 2.1 | 102.1 | 16.8 | 286 | 0.5 | 3.0 | 103.0 | 16.3 | 366 | 0.0 | 0.2 | 100.2 | 260.9 |
| iv biwk + TV-1011 | 2 | 22.3 | 152 | 2.6 | 13.4 | 113.4 | 20.9 | 140 | 1.2 | 8.3 | 108.3 | 19.3 | 103 | −0.3 | −1.5 | 98.5 | −8.6 |
| 30 mg/kg qd*5, then | 3 | 22.1 | 238 | 3.6 | 19.4 | 119.4 | 21.6 | 294 | 3.2 | 17.1 | 117.1 | 21.6 | 610 | 3.1 | 17.0 | 117.0 | 494.5 |
| 40 mg/kg biwk | 4 | | | | | | | | | | | | | | | | |
| | 5 | 22.1 | 263 | 2.3 | 11.5 | 111.5 | 21.9 | 274 | 2.2 | 10.9 | 110.9 | 22.0 | 510 | 2.2 | 11.2 | 111.2 | 389.7 |
| | 6 | 22.6 | 275 | 1.0 | 4.4 | 104.4 | 22.7 | 354 | 1.1 | 5.0 | 106.0 | 22.3 | 945 | 0.7 | 3.0 | 103.0 | 820.2 |
| | 7 | 23.1 | 401 | 2.9 | 14.5 | 114.5 | 22.1 | 530 | 1.9 | 9.4 | 109.4 | 21.7 | 655 | 1.5 | 7.5 | 107.5 | 518.1 |
| | 8 | 20.7 | 238 | 1.7 | 9.0 | 109.0 | 21.9 | 351 | 2.9 | 15.5 | 115.5 | 21.0 | 947 | 2.0 | 10.7 | 110.7 | 801.9 |
| | 9 | 20.4 | 192 | 0.8 | 4.1 | 104.1 | 20.2 | 253 | 0.5 | 2.8 | 102.8 | 19.7 | 414 | 0.1 | 0.3 | 100.3 | 264.9 |
| | 10 | 21.7 | 376 | 2.8 | 14.7 | 114.7 | 21.8 | 620 | 2.8 | 15.0 | 115.0 | 20.2 | 1096 | 1.2 | 8.4 | 106.4 | 924.5 |
| | Mean | 21.3 | 255.0 | 2.0 | 10.4 | 110.4 | 21.1 | 344.8 | 1.8 | 9.4 | 109.4 | 20.4 | 627.1 | 1.2 | 6.1 | 106.1 | 496.1 |
| | n | 9 | 9 | 9 | 9 | 9 | 9 | 9 | 9 | 9 | 9 | 9 | 9 | 9 | 9 | 9 | 9 |
| | SD | 1.9 | 87.1 | 1.1 | 5.8 | 5.8 | 1.8 | 146.5 | 1.0 | 5.5 | 5.5 | 1.9 | 321.2 | 1.2 | 6.2 | 6.2 | 307.8 |
| | SE | 0.6 | 29.0 | 0.4 | 1.9 | 1.9 | 0.6 | 48.8 | 0.3 | 1.8 | 1.8 | 0.6 | 107.1 | 0.4 | 2.1 | 2.1 | 102.6 |
| 7. Velcade ® 0.5 mg/kg | 1 | 22.2 | 478 | 1.0 | 4.6 | 104.6 | 22.0 | 927 | 0.8 | 3.7 | 103.7 | 22.0 | 1585 | 0.7 | 3.4 | 103.4 | 1478.8 |
| iv biwk + TV-1011 | 2 | 23.5 | 194 | 2.7 | 13.2 | 113.2 | 23.1 | 305 | 2.3 | 11.2 | 111.2 | 21.8 | 465 | 1.0 | 4.6 | 104.6 | 353.2 |
| 30 mg/kg qd*5, then | 3 | 22.2 | 262 | 3.9 | 21.2 | 121.2 | 21.9 | 454 | 3.5 | 19.2 | 119.2 | 21.9 | 1039 | 3.6 | 19.5 | 119.5 | 923.7 |
| 30 mg/kg biwk | 4 | 20.2 | 344 | 1.8 | 9.5 | 109.5 | 20.7 | 337 | 2.2 | 12.1 | 112.1 | 19.9 | 819 | 1.5 | 7.9 | 107.9 | 699.8 |
| | 5 | 21.0 | 168 | 2.9 | 16.2 | 116.2 | 20.7 | 191 | 2.6 | 14.6 | 114.6 | 20.0 | 461 | 1.9 | 10.7 | 110.7 | 340.4 |
| | 6 | 19.2 | 311 | 0.9 | 5.1 | 105.1 | 20.1 | 512 | 1.8 | 10.0 | 110.0 | 18.6 | 764 | 0.3 | 1.4 | 101.4 | 636.6 |
| | 7 | 21.0 | 228 | 0.7 | 3.2 | 103.2 | 21.6 | 438 | 1.2 | 5.9 | 105.9 | 20.9 | 685 | 0.6 | 2.7 | 102.7 | 554.9 |
| | 8 | 21.7 | 176 | 1.6 | 7.9 | 107.9 | 22.1 | 260 | 2.0 | 9.8 | 109.8 | 22.1 | 475 | 2.0 | 9.8 | 109.8 | 328.6 |
| | 9 | 21.8 | 547 | 3.0 | 16.1 | 116.1 | 21.8 | 730 | 3.0 | 16.0 | 116.0 | 21.6 | 1393 | 2.8 | 14.8 | 114.8 | 1244.2 |
| | 10 | 20.9 | 546 | 1.8 | 9.3 | 109.3 | 21.0 | 646 | 1.9 | 9.9 | 109.9 | 20.4 | 1028 | 1.3 | 6.7 | 106.7 | 862.5 |
| | Mean | 21.4 | 327.3 | 2.0 | 10.6 | 110.6 | 21.5 | 479.9 | 2.1 | 11.2 | 111.2 | 20.9 | 871.4 | 1.5 | 8.1 | 108.1 | 742.3 |
| | n | 10 | 10 | 10 | 10 | 10 | 10 | 10 | 10 | 10 | 10 | 10 | 10 | 10 | 10 | 10 | 10 |
| | SD | 1.2 | 148.2 | 1.1 | 5.9 | 5.9 | 0.9 | 230.0 | 0.8 | 4.6 | 4.6 | 1.2 | 390.6 | 1.0 | 5.7 | 5.7 | 390.7 |
| | SE | 0.4 | 46.9 | 0.3 | 1.9 | 1.9 | 0.3 | 72.7 | 0.3 | 1.5 | 1.5 | 0.4 | 123.5 | 0.3 | 1.8 | 1.8 | 123.5 |
| 8. TV-1011 30 mg/kg | 1 | 23.2 | 101 | 1.8 | 8.5 | 108.5 | 22.1 | 106 | 0.7 | 3.3 | 103.3 | 22.7 | 92 | 1.3 | 5.9 | 105.9 | −14.3 |
| qd*5, 40 mg/kg biwk | 2 | 20.6 | 360 | 3.0 | 16.7 | 116.7 | 20.7 | 638 | 3.0 | 16.7 | 116.7 | 21.5 | 955 | 3.9 | 21.8 | 121.8 | 842.5 |
| | 3 | 23.1 | 268 | 2.6 | 12.9 | 112.9 | 22.4 | 463 | 2.0 | 9.7 | 109.7 | 22.8 | 891 | 2.3 | 11.4 | 111.4 | 775.4 |
| | 4 | 19.1 | 267 | 3.3 | 20.7 | 120.7 | 19.5 | 399 | 3.6 | 22.9 | 122.9 | 18.8 | 568 | 3.0 | 18.7 | 118.7 | 448.8 |
| | 5 | 21.6 | 556 | 2.0 | 10.1 | 110.1 | 20.2 | 767 | 0.6 | 3.1 | 103.1 | 20.8 | 1324 | 1.2 | 6.2 | 106.2 | 1200.9 |
| | 6 | 21.8 | 581 | 1.5 | 7.6 | 107.6 | 21.1 | 663 | 0.8 | 4.0 | 104.0 | 21.7 | 804 | 1.4 | 7.1 | 107.1 | 676.2 |
| | 7 | 20.5 | 211 | 2.6 | 14.4 | 114.4 | 19.0 | 257 | 1.1 | 6.1 | 106.1 | 19.1 | 379 | 1.2 | 6.8 | 106.8 | 247.2 |
| | 8 | 20.5 | 257 | 3.9 | 23.3 | 123.3 | 19.0 | 246 | 2.3 | 14.0 | 114.0 | 19.8 | 273 | 3.2 | 19.0 | 119.0 | 135.4 |
| | 9 | 23.3 | 451 | 3.2 | 15.7 | 115.7 | 21.8 | 638 | 1.7 | 8.4 | 108.4 | 22.6 | 1001 | 2.5 | 12.4 | 112.4 | 853.6 |
| | 10 | 19.3 | 392 | 1.5 | 8.4 | 108.4 | 18.4 | 588 | 0.7 | 3.7 | 103.7 | 18.9 | 742 | 1.1 | 6.3 | 106.3 | 570.4 |
| | Mean | 21.3 | 344.4 | 2.5 | 13.8 | 113.8 | 20.4 | 476.6 | 1.7 | 9.2 | 109.2 | 20.9 | 702.9 | 2.1 | 11.5 | 111.5 | 573.6 |
| | n | 10 | 10 | 10 | 10 | 10 | 10 | 10 | 10 | 10 | 10 | 10 | 10 | 10 | 10 | 10 | 10 |
| | SD | 1.6 | 153.2 | 0.8 | 5.4 | 5.4 | 1.4 | 218.0 | 1.1 | 6.7 | 6.7 | 1.6 | 375.1 | 1.0 | 6.2 | 6.2 | 373.4 |
| | SE | 0.5 | 48.4 | 0.3 | 1.7 | 1.7 | 0.5 | 68.9 | 0.3 | 2.1 | 2.1 | 0.5 | 118.6 | 0.3 | 1.9 | 1.9 | 118.1 |

Discussion

Clusterin expression is upregulated in many tumors tested, and sCLU is a cytoprotective protein that is upregulated in response to tumor cell killing interventions, such as chemotherapy, hormone ablative, and radiation therapies. In response to anti-tumor interventions, sCLU prevents apoptosis by interacting with the stressed cell surface proteins to inhibit pro-apoptotic signal transduction. In addition, it interacts with the altered conformation of Bax to inhibit apoptosis. Furthermore, sCLU increases Akt phosphorylation and cell survival, which in turn is promoted by the increase of NF-κB nuclear transactivation by clusterin (Zoubeidi et al., 2010).

A broad range of in vitro studies have shown that custirsen potently regulated the expression of clusterin, facilitated apoptosis and sensitized cancerous human prostate, breast, ovarian, non small cell lung cancer, renal, bladder, and melanoma cells to chemotherapy, thus inhibition of clusterin may enhance the effects of drug therapies in the treatment of cancer (Zoubeidi and Gleave 2012). Custirsen is a second-generation ASO, is designed to specifically bind to a portion of the clusterin mRNA, resulting in the inhibition of the production of the human secreted isoform of clusterin (sCLU).

Secretary clusterin (sCLU)-2 is a stress-activated, cytoprotective chaperone that confers broad-spectrum cancer treatment resistance and its targeted inhibitor (TV-1011, Custirsen) is currently in Phase III trials for prostate cancer. Custirsen inhibits the production of clusterin, a protein that is associated with treatment resistance in a number of solid tumors and hematological cancer, including human myeloma (plasmacytoma, B cells) along with prostate, breast, non-small cell lung, ovarian, and bladder cancers. It has potential applicability as a therapeutic in a broad number of cancers at different stages and can potentially be used in combination with a variety of commonly used cancer treatments, including chemotherapy, radiation therapy, and hormone ablation therapy.

In clinical settings, the primary tolerability issues for second-generation ASOs, such as custirsen, have proven to be constitutional symptoms such as pyrexia, chills, and headaches, phenomena secondary to nonhybridization-dependent effects, as observed in Study OGX 011 01, where a dose relationship pattern was seen with an increased incidence of these constitutional symptoms in the higher dose cohorts. In addition, other laboratory abnormalities such as prolongation of activated partial thromboplastin time (aPTT), elevation of liver enzymes, and decrease in platelet count have also been reported; however, as stated above, second-generation ASOs have shown a better safety profile than the first-generation ASOs (Gleave et al., 2002; Akdim et al., 2010; Glover at al., 1997; Sewell et al., 2002). Similar results were observed in another study, where ISIS 104838, a 2′MOE ASO, was administered to healthy volunteers (HVS). Although the drug was generally well tolerated, transient aPTT prolongation occurred for the highest dose of 6 mg/kg at the maximal concentration, resolving a few hours afterwards with no evidence of compromised hemostasis (Sewell et al., 2002).

In a Phase I dose-escalation study utilizing doses from 40 mg to 640 mg of custirsen (Study OGX 011 01), prostate cancer patients were administered repeated doses of custirsen in combination with neoadjuvant hormone therapy. In this study, no premedication was administered to any patients, but the patients were allowed to take acetaminophen, nonsteroidal anti inflammatory drugs (NSAIDs), codeine, or narcotic-based pain medication.

No dose limiting toxicities, deaths, or adverse events (AEs) leading to withdrawal were reported in this study. Some of the more common events, such as chills, fatigue, pyrexia, and headache, were observed more frequently in the higher dose groups, which suggests a dose relationship. Hepatic transaminase elevations of Grade 1 and 2 were observed, particularly in higher dose groups. This study showed that constitutional symptoms associated with custirsen administration appeared to be dose dependent and occurred almost exclusively within the loading-dose week.

In a Phase I HV study with custirsen (TV1011-DDI-102), subjects either received a single dose of 640 mg custirsen with a premedication regimen of 400 mg ibuprofen or repeated doses of 640 mg custirsen with premedication regimen of 650 mg acetaminophen. Similar constitutional symptoms, as with the above-mentioned study, were experienced by subjects, including upper abdominal pain, arthralgia, asthenia, back pain, chest discomfort, chills, dizziness, feeling cold, feeling hot, headaches, hyperhidrosis, myalgia, nausea, pruritus, pyrexia, and vomiting. Symptoms were only partially alleviated by ibuprofen or acetaminophen administration. Three subjects withdrew from the study due to these constitutional symptoms. No deaths or other SAEs were reported.

This study showed that both single and repeated doses of 640 mg custirsen resulted in no SAEs, but the tolerability of custirsen was poor in healthy subjects. Acetaminophen or ibuprofen premedication only partially aided in custirsen tolerability. In the group that received multiple 640 mg custirsen doses, the number of AEs that were experienced decreased as well as the number of subjects who experienced the AEs. For example, after the first infusion of custirsen 19 subjects reported 93 AEs, which were considered to be related to the administration of custirsen. However, after the second infusion of custirsen, 12 subjects reported 79 AEs, which were attributed to the administration of custirsen, and only 2 subjects reported 2 AEs attributed to the administration of custirsen after the third infusion (Study TV1011-DDI-102).

Other than a possible effect on pulse, dexamethasone has no safety implication in the context of the study herein (i.e., low dose administration over 4 custirsen doses) and is therefore considered an appropriate pretreatment for custirsen. Dexamethasone premedication appears to ameliorate the constitutional side effects of custirsen and there does not appear to be a need to increase the dose of dexamethasone with up-titration of custirsen. Analysis of the ECG Holter data suggests no interruption of normal cardiac conduction and all measured interval durations were maintained within normal values. Somewhat unexpectedly, an impact on heart rate is observed. There may be role for dexamethasone in triggering tachycardia and a possible role for custirsen in modulating or contributing to it. It may also be that the heart rate increase is part of the constitutional symptoms syndrome of custirsen, which may be successfully mitigated through the up-titration regimen disclosed herein. Heart rate increase is not a known effect of dexamethasone.

Safety data in humans are available from 7 completed studies evaluating custirsen used in combination with NHT, docetaxel or gemcitabine in combination with a platinum-based regimen.

The most common adverse events (occurring in more than 20% of the patients) included fatigue, chills, nausea, pyrexia, anorexia, diarrhea, constipation, vomiting, alopecia, edema peripheral, dyspnoea, dysgeusia, cough, insomnia, arthralgia, myalgia, hot flush, peripheral sensory neuropathy, headache, dizziness, and bone pain.

During the loading-dose week, when custirsen is given alone, patients commonly experienced fever and chills/rigors. In Study OGX-011-03, fever occurred in 50% of patients, and chills in 35% of patients. These events were usually mild (grade 1-2), appear 4-8 hours after the infusion, and responded to pre-medication with NSAIDS. In general, occurrence of these symptoms was not associated with early termination from the study in cancer patients, but was less tolerable in healthy subjects during Study TV1011-DDI-102.

As disclosed herein, custirsen up-titration with or without premedication with dexamethasone is found to be effective in decreasing the severity and the intensity of the constitutional syndrome.

PTT Prolongation

Coagulation studies were assessed in both Phase 1 Studies OGX-011-01 and OGX-011-02. In both studies there were no INR values above 1.5. In Study OGX-011-01 there was only 1 grade 1 increase in PTT at the 320-mg dose. In Study OGX-011-02 there were only 2 reports of grade 2 toxicity for PTT, one in the 360-mg arm and 1 in the 640-mg arm.

In addition there was no significant incidence of hemorrhagic events in the overall treatment emergent data. In the OGX-011-03 study the incidence of hemorrhagic events was similar between the arms. A trend of abnormality in coagulation tests was also evident in Study TV1011-DDI-102. Slightly prolonged coagulation parameters were seen in most subjects (PTT and PT), starting a few hours after the first loading dose and peaking around 48 hours after dose. At its peak, International Normalized Ratio (INR) did not exceed 1.5, and no clinical symptoms such as bleeding or hematomas were associated with this increase. All patients spontaneously returned to their baseline. This abnormality was evident only in Study TV1011-DDI-102 study due to close monitoring of laboratory tests on the first days after the dose. Overall, abnormal coagulation seems to be limited in duration and severity, reversible, and of no clinical significance.

Because clinical experience with second-generation ASOs is limited, other events or more severe events which are not currently predictable could occur.

Elevated Liver Function Test

A potential for liver toxicity has been observed from both toxicity studies with other ASOs and from nonclinical studies with custirsen sodium. A higher incidence of elevated liver function tests (ELFTs) was seen only in Studies OGX-011-01 and OGX-011-04 when custirsen was administered with the neoadjuvant hormone therapy (NBT) flutamide. Study OGX-011-04 was amended in order to not allow treatment with flutamide; after the amendment there were no reports of grade-4 liver toxicity. The occurrence of grade 3/4 hepatic toxicity was rare in Study OGX-011-03.

Elevated Creatinine Levels

A higher incidence of elevated creatinine (primarily grade 1/2) was reported in the randomized Phase 2 study (Study OGX-011-03) for the custirsen plus docetaxel arm (23%) compared to the docetaxel arm (5%). All elevated creatinine levels were transient. The occurrence of grade 3/4 renal toxicity was rare in the Phase 1 and 2 studies. As a safety precaution, creatinine levels are monitored during Study OGX-011-11.

Lymphopenia

The incidence of lymphopenia was higher for custirsen treated patients in combination with chemotherapy compared with patients treated with chemotherapy alone as seen in Study OGX-011-03. Lymphopenia was seen in 78% of patients, with grade 3/4 in 29% of patients. However, there were no clinical sequela such as a higher infection rates as a result of lymphopenia. Complete blood counts including lymphocyte counts, are being performed during Study OGX-011-11, which enable monitoring lymphocyte levels as safety precaution.

Dexamethasone

Common adverse events experienced by patients receiving dexamethasone injection include nausea, vomiting, dyspepsia, appetite change, edema, headache, dizziness, mood swings, insomnia, anxiety, hypokalemia, hypertension, hyperglycemia, Cushing syndrome (long-term use), menstrual irregularities, ecchymosis, acne, skin atrophy (long-term use), and impaired wound healing (long-term use).

Serious adverse events reported in the literature include adrenal insufficiency, steroid psychosis, immunosuppression (long-term use), peptic ulcer, congestive heart failure, anaphylaxis, osteoporosis (long-term use), pseudotumor cerebri (pediatric patients), and pancreatitis (pediatric patients).

In Example 3, the 5-mg dose of dexamethasone being administered is very low, there is no chronic administration and pharmacodynamic washout is complete before the next dexamethasone dose is given. No special measures are needed and no serious adverse events associated with dexamethasone administration are expected in Example 3.

Anti-clusterin oligonucleotides potently reduce the expression of clusterin and sensitize cancerous human prostate, breast, ovarian, non small cell lung cancer, renal, bladder, myeloma, and melanoma cells to chemotherapy. Anti-clusterin oligonucleotides also enhance the effects of therapies such as radiation and hormone ablation in the treatment of certain cancers. Combination therapies comprising anti-clusterin oliconucleotides are described in Zoubeidi and Cleave 2012, U.S. Pat. Nos. 7,534,773; 7,368,436; 7,569,551; 7,592,323; 7,732,422; 6,900,187; 7,285,541; and 8,361,981, and U.S. Patent Application Publication Nos. 2011/0142827; 2008/119425; and 2013/0017272, the entire contents of each of which are hereby incorporated herein by reference. However, as disclosed herein, the three loading doses of 640 mg used for treatment can result in flu-like side effects such as fever, chills, and rigors. No method for overcoming the side effects of the custirsen loading doses has been identified.

Surprisingly, by reducing at least one of the three loading doses of custirsen to be less than 640 mg, side effects such as fever and chills/rigors are mitigated. Additionally, custirsen is still effective for cancer treatment after such a reduction. Therefore, the present invention provides an effective dosing regimen that reduces side-effects compared to the dosing strategy that is currently used for custirsen.

### REFERENCES

Abrams M T, Koser M L, Seitzer J, Williams S C, DiPietro M A, Wang W, Shaw A W, Mao X, Jadhav V. Davide J P, Burke P A, Sachs A B, Stirdivant S M, Sepp-Lorenzino L. Evaluation of Efficacy, Biodistribution and Inflammation for a Potent siRNA Nanoparticle: Effect of Dexamethasone Co-treatment. Molecular Therapy (2010) 18 1, 171-180.
Abreu-Martin M T, Chari A, Palladino A A, et al. Mitogen-activated protein kinase kinase kinase 1 activates androgen receptor-dependent transcription and apoptosis in prostate cancer. Mol Cell Biol 1999; 19(7):5143-54.
Akdim F, Stroes E S, Sijbrands E J, Tribble D L, Trip M D, Jukema J W, et al. Efficacy and safety of mipomersen, an antisense inhibitor of apolipoprotein B, in hypercholesterolemic subjects receiving stable statin therapy. J Am Coll Cardiol. 2010 Apr. 13; 55(15):1611-1618.
Bennett C F, Swayze E E. RNA targeting therapeutics: molecular mechanisms of antisense oligonucleotides as a therapeutic platform. Annu Rev Pharmacol Toxicol. 2010; 50:259-293.
Craft N, Shostak Y, Carey M, at al. A mechanism for hormone-independent prostate cancer through modulation of androgen receptor signaling by the HER-2/neu tyrosine kinase. Nat Med 1999; 5(3):280-5.
Crooke T. S., Antisense Drug Technologies; Principles, Strategies and Applications; 2007, Chapter 13, CRS Press.
Custirsen Sodium Investigator's Brochure (IB), Ed 8.0, May 2011.
Demolis J L, Kubitza D, Tenneze L, Funck-Brentano C. Effect of a single oral dose of moxifloxacin (400 mg and 800 mg) on ventricular repolarization in healthy subjects. Clin Pharmacol Ther 2000; 68:658-66.
Denis L, Murphy G P. Overview of phase III trials on combined androgen treatment in patients with metastatic prostate cancer. Cancer 1993; 72(12 Suppl):3888-95.
Drug Monograph: Dexamethasone Injection. Drugs.com, revised December 2009 Available at: www.drugs.com/pro/dexamethasone-injection.html (Accessed on Nov. 8, 2012).
Eisenberger M A, Blumenstein B A, Crawford E D, et al. Bilateral orchiectomy with or without flutamide for metastatic prostate cancer. N Engl J Med 1998; 339(15):1036-42.
Gleave M E, Bruchovsky N, Moore M J, et al. Prostate cancer: 9. Treatment of advanced disease. Cmaj 1999; 160(2):225-32.
Gleave M, Miyake H, Zangemeister-Wittke U, et al. Antisense therapy: current status in prostate cancer and other malignancies. Cancer Metastasis Rev 2002; 21(1):79-92.
Glover J M, Leeds J M, Mant T G, Amin D, Kisner D L, Zuckerman J E, et al. Phase I safety and pharmacokinetic profile of an intercellular adhesion molecule-1 antisense oligodeoxynucleotide (ISIS 2302). J Pharmacol Exp Ther. 1997 September; 282(3):1173-1180.
Henry S. Stecker K, Brooks D, et al. Chemically modified oligonucleotides exhibit decreased immune stimulation in mice. J Pharmacol Exp Ther 2000; 292(2):468-79.
Franchimont D, Louis E, Dewe W, Martens H, Vrindts-Gevaert Y, De Groote D, Belaiche J, Geenen V. Effects of dexamethasone on the profile of cytokine secretion in human whole blood cell cultures. Regul Pept. 1998 Jan. 2; 73(1) 59-65.
Fridericia L S. Die Systolendauer im Elektrokardiogramm bei normalen Menchen und bei Herzkranken. Acta Med Scand 1920:53; 469-86.
Hara I, Miyake H, Gleave M E, et al. Introduction of clusterin gene into human renal cell carcinoma cells enhances their resistance to cytotoxic chemotherapy through inhibition of apoptosis both in vitro and in vivo. Jpn J Cancer Res 2001; 92(11):1220-4.
Miyake H, Nelson C, Rennie P S, et al. Testosterone-repressed prostate message-2 is an antiapoptotic gene involved in progression to androgen independence in prostate cancer. Cancer Res 2000; 60(1):170-6.
Miyake H, Hara S, Zellweger T, et al. Acquisition of resistance to Fas-mediated apoptosis by overexpression of clusterin in human renal-cell carcinoma cells. Mol Urol 20015(3):105-11.
Miyake H, Chi K N, Gleave M E. Antisense TRPM-2 oligodeoxynucleotides chemosensitize human androgen-independent PC-3 prostate cancer cells both in vitro and in vivo. Clin Cancer Res 2000; 6(5):1655-63.
Miyake H, Eto H, Hara I, et al. Synergistic antitumor activity by combined treatment with gemcitabine and antisense oligodeoxynucleotide targeting clusterin gene in an intravesical administration model against human bladder cancer KOTCC-1 cells. J Urol 2004; 171(6 Pt 1):2477-81.
Miyake H, Gleave M, Kamidono 5, et al. Overexpression of clusterin in transitional cell arcinoma of the bladder is related to disease progression and recurrence. Urology 2002; 59(1):150-4.
Miyake H, Nelson C, Rennie P S, at al. Acquisition of chemoresistant phenotype by overexpression of the antiapoptotic gene testosterone-repressed prostate message-2 in prostate cancer xenograft models. Cancer Res 2000; 60(9):2547-54.
Miyake H, Hara I, Kamidono S, et al. Synergistic chemsensitization and inhibition of tumor growth and metastasis by the antisense oligodeoxynucleotide targeting clusterin gene in a human bladder cancer model. Clin Cancer Res 2001; 7(12):4245-52.
Noel G J, Natarajan J, Chien S, Hunt T L, Goodman D B, Abels R. Effects of three fluoroquinolones on QT interval in healthy adults after single doses. Clin Pharmacol Ther 2003; 73:292-303.
Parker S L, Tong T, Bolden S, et al. Cancer statistics, 1996. CA Cancer J Clin 1996; 46(1): δ 27.
Redondo M, Villar E, Torres-Munoz J, at al. Overexpression of clusterin in human breast carcinoma. Am J Pathol 2000157(2):393-9.
Sewell K L, Geary R S, Baker B F, Glover J M, Mant T G, Yu R Z, et al. Phase I trial of ISIS 104838, a 2′-methoxyethyl modified antisense oligonucleotide targeting tumor necrosis factor-alpha. J Pharmacol Exp Ther. 2002 December; 303(3):1334-1343.
Shah R R. Drugs, QTc interval prolongation and final ICH E14 guideline: an important milestone with challenges ahead. Drug Safety 200528(11):1009-28.
Sherwood E R, Van Dongen J L, Wood C G, et al. Epidermal growth factor receptor activation in androgen-independent but not androgen-stimulated growth of human prostatic carcinoma cells. Br J Cancer 1998; 77(6):855-61.
Sowery R D, Hadaschik B A, So A I, at al. Clusterin knockdown using the antisense oligonucleotide OGX-011 re-sensitizes docetaxel-refractory prostate cancer PC-3 cells to chemotherapy. BJU Int 2008; 102(3):389-97.
Study TV101′-DDI-102. Draft Clinical Trial Report, Version 3.0. 19 Mar. 2012.
TV1011-PD-107-PK Bioanalytical Report “Determination of Custirsen in Human Plasma Samples from TV1011-PD-107 Study”. Final Version. October 2012.
TV1011-PD-107-PK Urine Bioanalysis Report “Analysis of TV-1011 and N-1 to N-15 Metabolites in Human Urine Samples from the Study Titled “A Two Group, Open-label, Crossover Study to Evaluate the Effect of Custirsen Dosing Regimen on the Tolerability and ECG in Healthy Male Volunteers” by Capillary Gel Electrophoresis with Ultra-violet Detection (CGE UV)”. Final Version. 12 Oct. 2012.
Tsikouris J P, Pesters M J, Cox C D, Meyerrose G E, Seifert C F. Effects of three fluoroquinolones on QT analysis after standard treatment courses. Ann Noninvasive Electrocardiol 2006; 11(1):52 6.
TV1011-PD107-ECG Cardiac Safety Report for TV1011-PD-107. Final Version 1. 10 Sep. 2012.
T. Jesse Kwoh. “CHAPTER 13: An Overview of the Clinical Safety Experience of First- and Second-Generation Antisense Oligonucleotides.” Antisense Drug Technology; Principals, Strategies and Applications. Edited by Stanley T Crooke (Isis Pharmaceuticals, Inc). Carlsbad Calif. Marcel Dekker Inc. 2001. New York.
Wellmann A, Thieblemont C, Pittaluga S, et al. Detection of differentially expressed genes in lymphomas using cDNA arrays: identification of clusterin as a new diagnostic marker for naplastic large-cell lymphomas. Blood 2000; 96(2):398-404.
Yacyshyn B R, Barish C, Goff J, Dalke D, Gaspan M, Yu R, et al. Dose ranging pharmacokinetic trial of high-dose alicaforsen (inter cellular adhesion molecule-1 antisense oligodeoxynucleotide) (ISIS 2302) in active Crohn's disease. Alimentary Pharmacology & Therapeutics 2002; 16(10):1761-70.
Zellweger T, Miyake H, July L V, et al. Chemosensitization of human renal cell cancer using antisense oligonucleotides targeting the antiapoptotic gene clusterin. Neoplasia 2001; 3(4):360-7.
Zellweger T, Chi K, Miyake H, at al. Enhanced radiation sensitivity in prostate cancer by inhibition of the cell survival protein clusterin. Clin Cancer Res 2002; 8(10):3276-84.
Zoubeidi A, Chi K, Gleave M. Targeting the cytoprotective chaperone, clusterin, for treatment of advanced cancer. Clin Cancer Res 2010; 16(4):1088-93
Zoubeidi A and Gleave M. Small heat shock proteins in cancer therapy and prognosis. Int J Biochem Cell Biol 2012; 44(10):1646-56.

1. A method for providing antisense therapy which reduces the expression of clusterin to provide therapeutic benefit in the treatment of cancer, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, to a human subject in need of treatment for the cancer, which human subject also receives at least one chemotherapeutic agent, hormone ablation therapy, or radiation therapy, wherein the anti-clusterin oligonucleotide is administered at least 3 times during a 5 to 9 day period, wherein at least 1 of the administrations is at a dose other than 640 mg.

2. The method of claim 1, wherein the 5-9 day period is a 7 day period.

3. The method of claim 3, wherein the anti-clusterin oligonucleotide is administered on day 1, 3 and 5 of the 7 day period.

4. The method of claim 3, wherein the 7 day period is immediately before the first week of a chemotherapy treatment cycle.

5. The method of claim 1, wherein the anti-clusterin oligonucleotide is administered to the subject 3 times within a 5 to 9 day period and then once weekly thereafter.

6. The method of claim 1, wherein about 640 mg of the anti-clusterin oligonucleotide is administered to the human subject once in the 5 to 9 day period.

7. The method of claim 1, wherein about 640 mg of the anti-clusterin oligonucleotide is administered to the human subject twice in the 5 to 9 day period.

8. The method of claim 1, wherein about 480 mg of the anti-clusterin oligonucleotide is administered to the human subject once in the 5 to 9 day period.

9. The method of claim 1, wherein about 480 mg of the anti-clusterin oligonucleotide is administered to the human subject twice in the 5 to 9 day period.

10. The method of claim 1, wherein about 480 mg of the anti-clusterin oligonucleotide is administered to the human subject 3 times in the 5 to 9 day period.

11. The method of claim 1, wherein about 320 mg of the anti-clusterin oligonucleotide is administered to the human subject once in the 5 to 9 day period.

12. The method claim 1, wherein about 320 mg of the anti-clusterin oligonucleotide is administered to the human subject twice in the 5 to 9 day period.

13. The method of claim 1, wherein about 320 mg of the anti-clusterin oligonucleotide is administered to the human subject 3 times in the 5 to 9 day period.

14. The method of claim 1, wherein the anti-clusterin oligonucleotide is administered at 1 dose of 320 mg, 1 dose of 480 mg, and 1 dose of 640 mg, in the 5 to 9 day period.

15. The method of claim 14, wherein the first, second and third doses in the 5 to 9 day period are 320, 480, and 640 mg, respectively.

16. The method of claim 1, wherein the doses are administered on days 1, 3 and 5 of the 5 to 9 day period.

17. The method of claim 1, wherein the human subject receives a 320-mg dose of the anti-clusterin oligonucleotide on day 1, a 480-mg dose of the anti-clusterin oligonucleotide on day 3, and a 640-mg dose of the anti-clusterin oligonucleotide on day 5 of the 5 to 9 day period.

18-71. (canceled)

72. A method for providing antisense therapy which reduces the expression of clusterin to provide therapeutic benefit in the treatment of myeloma, comprising administering an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, to a human subject in need of treatment for myeloma.

73. The method of claim 72, wherein the human subject also receives at least one chemotherapeutic agent, hormone ablation therapy, or radiation therapy.

74. The method of claim 72, wherein the anti-clusterin oligonucleotide is administered at least 3 times during a 5 to 9 day period, wherein at least 1 of the administrations is at a dose other than 640 mg.

75-82. (canceled)
